(19) United States
(12) Patent Application Publication
(10) Pub. No.: US 2005/0058701 A1
(43) Pub. Date: Mar. 17, 2005

(54) Active drug delivery in the gastrointestinal tract

(76) Inventors: Yossi Gross, Moshav Mazor (IL); Yoram Sela, Ra'anana (IL); Ziv Belsky, Haifa (IL); Rina Lev, Haifa (IL); Daniel Goldstein, Efrat (IL)

Correspondence Address: RATNERPRESTIA, P O BOX 980, VALLEY FORGE, PA, 19482-0980, US

(21) Appl. No.: 10/901,742

(22) Filed: Jul. 29, 2004

Related U.S. Application Data
(63) Continuation-in-part of application No. 10/838,072, filed on May 3, 2004.
Application No. 10/838,072 is a continuation-in-part of application No. 10/767,663, filed on Jan. 29, 2004.
(60) Provisional application No. 60/443,173, filed on Jan. 29, 2003.

(51) Int. Cl. A61K 9/48; A61K 9/22

(52) U.S. Cl. 424/451; 604/890.1

(57) ABSTRACT

Apparatus for drug administration is provided, including an ingestible capsule, which includes a drug, stored by the capsule, and an environmentally-sensitive mechanism, adapted to change a state thereof responsively to a disposition of the capsule within a gastrointestinal (GI) tract of a subject. The capsule further includes first and second electrodes, and a control component, adapted to facilitate passage of the drug, in response to a change of state of the environmentally-sensitive mechanism, through an epithelial layer of the GI tract by driving the first and second electrodes to apply a series of pulses at a current of less than about 5 mA, at a frequency of between about 12 Hz and about 24 Hz, and with a pulse duration of between about 0.5 milliseconds and about 3 milliseconds.

### CROSS-REFERENCES TO RELATED APPLICATIONS

This application is a continuation-in-part of U.S. patent application Ser. No. 10/838,072, filed May 3, 2004, entitled, “Active drug delivery in the gastrointestinal tract,” which is a continuation-in-part of U.S. patent application Ser. No. 10/767,663, filed Jan. 29, 2004, entitled, “Active drug delivery in the gastrointestinal tract,” which claims the benefit of U.S. Provisional Patent Application 60/443,173, filed Jan. 29, 2003. All of these applications are assigned to the assignee of the present application and are incorporated herein by reference.

### FIELD OF THE INVENTION

The present invention relates to a gastrointestinal tract drug delivery system and, more particularly, to an ingestible drug-delivery facilitation system which enhances the absorption of a drug through the gastrointestinal wall. BACKGROUND OF THE INVENTION

The absorption of a drug (or of a drug precursor) into the systemic circulation is determined by the physicochemical properties of the drug, its formulations, and the route of administration, whether oral, rectal, topical, by inhalation, or by intravenous administration. Oral administration includes swallowing, chewing, sucking, as well as buccal administration, i.e., placing a drug between the gums and cheek, and sublingual administration, i.e., placing a drug under the tongue. A prerequisite to absorption is drug dissolution. Absorption of orally-administered drugs into the internal environment generally occurs almost exclusively in the small intestine. The small intestine is lined with a layer of epithelial cells joined by tight junctions. In order to pass from the lumen of the small intestine into the internal environment and, therefrom into the systemic circulation, a dissolved drug must either pass through the semi-permeable membranes of the epithelial cells (transcellular passage), or through the tight junctions between the epithelial cells. The rate of transcellular passage is generally low except for small, lipid-soluble molecules. In addition, the tight junctions generally prevent the passage of most dissolved molecules. A drug may cross the biological barrier by passive diffusion, or by other naturally-occurring transfer modes, for example, facilitated passive diffusion, active transport, or pinocytosis. Alternatively, a drug may be artificially assisted to cross the biological barrier.

In passive diffusion, transport depends on the concentration gradient of the solute across the biological barrier. Since the drug molecules are rapidly removed by the systemic circulation, drug concentration in the blood in the vicinity of the administration site is low compared with that at the administration site, producing a large concentration gradient. The drug diffusion rate is directly proportional to that gradient. The drug diffusion rate also depends on other parameters, for example, the molecule's lipid solubility and size. Because the cell membrane is lipoid, lipid-soluble drugs diffuse more rapidly than relatively lipid-insoluble drugs. Similarly, small drug molecules penetrate biological barriers more rapidly than large ones.

Another naturally occurring transfer mode is facilitated passive diffusion, which occurs for certain molecules, such as glucose. It is believed that a carrier component combines reversibly with a substrate molecule at the cell membrane exterior. The carrier-substrate complex diffuses rapidly across the membrane, releasing the substrate at the interior surface. This process is characterized by selectivity and saturability: The carrier is operative only for substrates with a relatively specific molecular configuration, and the process is limited by the availability of carriers.

Active transport, which is another naturally occurring transfer mode, appears to be limited to drugs that are structurally similar to endogenous substances. Active transport is characterized by selectivity and saturability and requires energy expenditure by the cell. It has been identified for various ions, vitamins, sugars, and amino acids.

Still another naturally occurring transfer mode is pinocytosis, in which fluids or particles are engulfed by a cell. The cell membrane encloses the fluid or particles, then fuses again, forming a vesicle that later detaches and moves to the cell interior. Like active transport, this mechanism requires energy expenditure. It is known to play a role in drug transport of protein drugs.

The foregoing discussion relates to naturally occurring transfer modes. Where these are insufficient, for example, in cases of macromolecules and polar compounds, which cannot effectively traverse the biological barrier, drug transport may be artificially induced.

Electrotransport refers generally to electrically induced passage of a drug (or a drug precursor) through a biological barrier. Several electrotransport mechanisms are known, as follows:

Iontophoresis involves the electrically induced transport of charged ions, by the application of low-level, direct current (DC) to a solution of the medication. Since like electrical charges repel, the application of a positive current drives positively charged drug molecules away from the electrode and into the tissues; similarly, a negative current will drive negatively charge ions into the tissues. Iontophoresis is an effective and rapid method of delivering water-soluble, ionized medication. Where the drug molecule itself is not water-soluble, it may be coated with a coating (for example, sodium lauryl sulfate (SLS)), that may form water-soluble entities.

Electroosmosis involves the movement of a solvent with the agent through a membrane under the influence of an electric field.

Electrophoresis is based on migration of charged species in an electromagnetic field. Ions, molecules, and particles with charge carry current in solutions when an electromagnetic field is imposed. Movement of a charged species tends to be toward the electrode of opposite charge. The voltages for continuous electrophoresis are rather high (several hundred volts).

Electroporation is a process in which a biological barrier is subjected to a high-voltage alternating-current (AC) surge, or pulse. The AC pulse creates temporary pores in the biological membrane. The pores allow large molecules, such as proteins, DNA, RNA, and plasmids to pass through the biological barrier.

Iontophoresis, electroosmosis, and electrophoresis are diffusion processes, in which diffusion is enhanced by electrical or electromagnetic driving forces. In contrast, electroporation physically punctures the biological barriers, along cell boundaries, enabling passage of large molecules through the epithelium. Generally, during electrotransport a combination of more than one of these processes occurs, together with passive diffusion and other naturally-occurring transfer modes. Therefore, electrotransport refers to at least one, and possibly a combination of the aforementioned transport mechanisms, which supplement the naturally-occurring transfer modes.

Medical devices that include drug delivery by electrotransport are described, for example, in U.S. Pat. No. 5,674,196 to Donaldson et al., U.S. Pat. No. 5,961,482 to Chien et al., U.S. Pat. No. 5,983,131 to Weaver et al., U.S. Pat. No. 5,983,134 to Ostrow, U.S. Pat. No. 6,477,410 to Henley et al., and U.S. Pat. No. 6,490,482 to Mori et al., all of whose disclosures are incorporated herein by reference.

In addition to the aforementioned electrotransport processes, there are other electrically assisted drug delivery mechanisms, including:

Sonophoresis, i.e., the application of ultrasound, induces growth and oscillations of air pockets, a phenomenon known as cavitation. These disorganize lipid bilayers thereby enhancing transport. For effective drug transport, a low frequency of between 20 kHz and less than 1 MHz, rather than the therapeutic frequency, should be used. Sonophoresis devices are described, for example, in U.S. Pat. Nos. 6,002,961, 6,018,678, and 6,002,961 to Mitragotri et al., U.S. Pat. Nos. 6,190,315 and 6,041,253 to Kost et al., U.S. Pat. No. 5,947,921 to Johnson et al., and U.S. Pat. Nos. 6,491,657 and 6,234,990 to Rowe et al., all of whose disclosures are incorporated herein by reference.

Ablation is another method of facilitating drug passage through a biological barrier. In addition to mechanical ablation, for example using hypodermic needles, ablation techniques include laser ablation, cryogenic ablation, thermal ablation, microwave ablation, radiofrequency ablation, liquid jet ablation, or electrical ablation.

U.S. Pat. No. 6,471,696 to Berube et al. describes a microwave ablation catheter, which may be used as a drug delivery device. U.S. Pat. No. 6,443,945 to Marchitto et al. describes a device for pharmaceutical delivery using laser ablation. U.S. Pat. No. 4,869,248 to Narula describes a catheter for performing localized thermal ablation, for purposes of drug administration. U.S. Pat. Nos. 6,148,232 and 5,983,135 to Avrahami describe drug delivery systems using electrical ablation. The disclosures of all of these patents are incorporated herein by reference.

Oral drug administration is a common drug delivery route. Drug bioavailability of orally administered drugs, i.e., the degree to which the drug is available to the target tissue, is affected by drug dissolution, drug degradation in the gastrointestinal (GI) tract, and drug absorption.

Drug dissolution is affected by whether the drug is in salt, crystal, or hydrate form. To improve dissolution, disintegrants and other excipients, such as diluents, lubricants, surfactants (substances which increase the dissolution rate by increasing the wettability, solubility, and dispersibility of the drug), binders, or dispersants are often added during manufacture.

Drug degradation in the GI tract is due to GI secretions, low pH values, and degrading enzymes. Since luminal pH varies along the GI tract, the drug must withstand different pH values. Interaction with blood, food staff, mucus, and bile may also affect the drug. Reactions that may affect the drug, and reduce bioavailability, include: (a) complex formations, for example, between tetracycline and polyvalent metal ions; (b) hydrolysis by gastric acid or digestive enzymes, for example, penicillin and chloramphenicol palmitate hydrolysis; (c) conjugation in the gut wall, for example, sulfoconjugation of isoproterenol; (d) adsorption to other drugs, for example, digoxin and cholestyramine; and (e) metabolism by luminal microflora.

Drug absorption of orally-administered drugs relates to transport of drugs across biological barriers presented by the epithelial cells in the GI tract. The nature of intestinal epithelium tends to inhibit drug absorption. As seen in FIG. 1 (based on Martinit, F. H., et al., Human Anatomy, Prentice Hall, Englewood Cliffs, N.J., 1995), the intestinal epithelium of the small intestine is formed as a series of finger-like projections, called intestinal villi. These are covered by columnar epithelium, carpeted with microvilli. The epithelial cells along the microvilli are strongly bound to each other, by tight junctions, also called the zona occludens. The tight junctions seal the internal environment of the body from the intestinal lumen. The size of gaps between tight junctions in humans is about 8 nm in the jejunum, and about 0.3 nm in the ileum and the colon. Therefore, particles with diameters greater than about 11.5 angstrom and/or several thousand daltons generally cannot penetrate the gaps.

Overall, low bioavailability is most common with oral dosage forms of poorly water-soluble, slowly absorbed drugs. Insufficient time in the GI tract is another common cause of low bioavailability. An ingested drug is exposed to the entire GI tract for no more than 1 to 2 days, and to the small intestine for only about 2 to 4 hours. If the drug does not dissolve readily or cannot penetrate the epithelial membrane quickly, its bioavailability will be low. Age, sex, activity, genetic phenotype, stress, disease (e.g., achlorhydria, malabsorption syndromes), or previous GI surgery can further affect drug bioavailability.

Table 1 below (from Encyclopedia of Controlled Drug Delivery, edited by Edith Mathiowitz) summarizes some parameters of the oral route that affect drug bioavailability.

TABLE 1

| | | Liquid | | Transit |
|---|---|---|---|---|
| | Area, | Secretion, | pH | Time, |
| Section | m² | liters/day | Value | hours |
| Oral | ˜0.05 | 0.5-2 | 5.2-6.8 | Short |
| cavity | | | | |
| Stomach | 0.1-0.2 | 2-4 | 1.2-3.5 | 1-2 |
| Duodenum | ˜0.04 | 1-2 | 4.6-6.0 | 1-2 |
| Small | 4500 | 0.2 | 4.7-6.5 | 1-10 |
| Intestine | (including | | | |
| | microvilli) | | | |
| Large | 0.5-1 | ˜0.2 | 7.5-8.0 | 4-20 |
| Intestine | | | | |

In addition to the physical barrier of the epithelial cells, chemical and enzymatic barriers affect drug absorption.

It is known to provide an ingestible capsule that includes a drug and a chemical that indirectly facilitates passage of the drug across the epithelial layer. For example, the chemical may induce a change in the epithelial layer that renders it transiently more permeable to the drug, whereupon the drug (indirectly facilitated by the action of the chemical), crosses the epithelial layer by diffusion.

Another important barrier to drug absorption is the pre-systematic, first-pass metabolism, primarily hepatic metabolism. The predominant enzymes in this metabolism are the multi-gene families of cytochrome P450, which have a central role in metabolizing drugs. It appears that variations in P450s between individuals lead to variations in their ability to metabolize the same drug.

Additionally, multidrug resistance (MDR) may be a barrier to drug absorption. MDR, which is a major cause of cancer treatment failure, is a phenomenon whereby cancer cells develop a broad resistance to a wide variety of chemotherapeutic drugs. MDR has been associated with overexpression of P-glycoprotein or multidrug resistance-associated protein (MRP), two transmembrane transporter molecules which act as pumps to remove toxic drugs from tumor cells. P-glycoprotein acts as a unidirectional efflux pump in the membrane of acute myeloid leukemia (AML) cells and lowers the intracellular concentration of cytotoxic agents, by pumping them out of leukemic cells. Yet it confers resistance to a variety of chemotherapy drugs, including daunorubicin.

Ingestible radio pills, which are ingestible capsules containing a transmitter and other electrical components are known. In 1964 researchers at Heidelberg University developed a pill for monitoring pH of the GI tract. (Noller, H. G., “The Heidelberg Capsule Used For the Diagnosis of Peptic Diseases,” Aerospace Medicine, February, 1964, pp. 115-117.) U.S. Pat. No. 4,844,076 to Lesho et al., issued July 1989, entitled, “Ingestible size continuously transmitting temperature monitoring pill,” whose disclosure is incorporated herein by reference, describes a temperature responsive transmitter, encapsulated in an ingestible size capsule. The capsule is configured to monitor average body temperature, internally. The ingestible size temperature pill can be configured in a rechargeable embodiment. In this embodiment the pill uses the inductive coil in the tank circuit as the magnetic pickup to charge a rechargeable nickel cadmium battery. U.S. Pat. No. 5,279,607 to Schentag et al., entitled, “Telemetry capsule and process,” whose disclosure is incorporated herein by reference, describes an ingestible capsule and a process for delivery, particularly repeatable delivery, of a medicament to the alimentary canal. The ingestible capsule is an essentially non-digestible capsule, which contains an electric energy emitting means, a radio signal transmitting means, a medicament storage means and a remote actuatable medicament releasing means. The capsule signals a remote receiver as it progresses through the alimentary tract in a previously mapped route and upon reaching a specified site is remotely triggered to release a dosage of medicament.

U.S. Pat. No. 5,395,366 to D'Andrea et al., entitled, “Sampling capsule and process,” whose disclosure is incorporated herein by reference, describes a similar ingestible capsule and a process for sampling of fluids in the alimentary canal.

The use of electrostimulating capsules for promoting peristalsis is known. PCT Publications WO 97/31679 to Dirin and WO 97/26042 to Terekhin, the disclosures of both of which are incorporated herein by reference, disclose ingestible capsules for electrostimulation of the alimentary tract, to be used, for example, as a post-surgical therapy, as a prophylactic measure of alimentary tract diseases, or for the promotion of peristalsis.

PCT Publication WO 97/31679 further discloses that USSR Inventor's Certificate No. 1223922, Int. Cl. A 61 N 1/36, Bulletin No. 14, by Pekarasky et al., entitled, “Gastrointestinal tract Electrostimulator,” which is incorporated herein by reference, describes a swallowable capsule adapted for electrostimulation of the alimentary tract, as post-surgical therapy, as a prophylactic measure of alimentary tract diseases, or for the promotion of peristalsis, which is further adapted for the dispensing of medication.

U.S. patent application 2003/0125788 to Long, which is incorporated herein by reference, describes a capsule for introduction into a bodily lumen. The capsule includes a balloon filled with a conductive fluid, or a mechanism for actuating wings supporting electrodes. An umbilicus may attach to the trailing end of the capsule. A control unit controls propulsion of the capsule through the bodily lumen.

U.S. patent application 2003/0093031 to Long, which is incorporated herein by reference, describes a drug-delivery system including: a capsule for introduction into a body lumen; an umbilicus attached to the capsule, which is flexible and of sufficient length to extend outside of the body lumen while the capsule is inside of the body lumen; and means for dispensing a medical agent into the lumen through the capsule. The capsule may include first and second electrodes. A channel may extend through the umbilicus to a plurality of weep holes in the capsule to fluidly connect the medical agent from outside the body lumen to the wall of the body lumen.

Methods of tracking ingestible devices, such as radio pills, are described, for example, in the above-mentioned U.S. Pat. No. Pat. 5,279,607 to Schentag et al., the above-mentioned U.S. Pat. Pat. 5,395,366 to D'Andrea et al., and U.S. Pat. No. 6,082,366 to Andrii et al., entitled, “Method and arrangement for determining the position of a marker in an organic cavity,” all of whose disclosures are incorporated herein by reference.

Visual examination of the GI tract by ingestible devices is known. U.S. Pat. No. 5,984,860 to Shan, entitled, “Pass-through duodenal enteroscopic device,” whose disclosure is incorporated herein by reference, describes a tethered ingestible, enteroscopic video camera, which utilizes the natural contraction wave of the small intestine to propel it through the small intestine at about the same speed as any other object therein. The video camera includes an illumination source at its forward end. Covering the camera lens and illumination source is a transparent inflatable balloon, adapted to gently expand the small intestine immediately forward the camera for better viewing. A small diameter communication and power cable unwinds through an aperture in the rear of the camera as it moves through the small intestine. Upon completion of movement through the small intestine the cable is automatically separated, permitting the cable to be withdrawn through the stomach and intestine. The camera continues through the large intestine and passes from the patient through the rectum.

U.S. Pat. No. 5,604,531 to Iddan et al., entitled, “In vivo video camera system,” whose disclosure is incorporated herein by reference, describes a video camera system, encapsulated within an ingestible capsule, arranged to pass through the entire digestive tract, operating as an autonomous video endoscope. The ingestible capsule includes a camera system and an optical system for imaging an area of interest onto the camera system, and a transmitter, which relays the video output of the camera system to an extracorporeal reception system. A light source is located within a borehole of the optical system.

Similarly, U.S. patent application 2001/0035902 to Iddan et al., entitled, “Device and system for in vivo imaging,” whose disclosure is incorporated herein by reference, describes a system and method for obtaining in vivo images. The system contains an imaging system and an ultra low power radio frequency transmitter for transmitting signals from a CMOS imaging camera to a receiving system located outside a patient.

Additionally, U.S. Pat. No. 6,428,469 to Iddan et al., entitled, “Energy management of a video capsule,” whose disclosure is incorporated herein by reference, describes an energy saving device for acquiring in vivo images of the gastro-intestinal tract. The device, such as an autonomous capsule, includes at least one imaging unit, a control unit connected to the imaging unit, and a power supply connected to the control unit. The control unit includes a switching unit, and an axial motion detector connected to the switching unit, which disconnects the power supply thereby preventing the acquisition of redundant images.

U.S. Pat. No. 6,632,216 to Houzego et al., which is incorporated herein by reference, describes an ingestible device for delivering a substance to a chosen location in the GI tract. The device includes a receiver of electromagnetic radiation for powering an openable part of the device to an opened position for dispensing of the substance. The receiver includes a coiled wire that couples the energy field, the wire having an air or ferrite core. The device optionally includes a latch defined by a heating resistor and a fusible restraint. The device may also include a flexible member that may serve one or both the functions of activating a transmitter circuit to indicate dispensing of the substance, and restraining of a piston used for expelling the substance.

PCT Publication WO 02/094369 to Walla, which is incorporated herein by reference, describes a device for applying substances such as medicaments having a liquid, ointment or gel-like consistency through the skin, especially by means of iontophoresis. The resorption of the substance occurs by application of a DC current. The publication also describes a capsular, hermetically sealed container for insertion into body orifices, which has at least two electrodes for generating a continuous electric field on its outer side. A device for receiving the substance to be applied is provided above the electrodes. The container is positioned to be in contact with the mucous membrane and/or the skin in a body orifice, especially in the urogenital, vaginal, and/or anal tract, and/or in the cavities of the mouth, ear, and/or nose.

U.S. Pat. No. 5,217,449 to Yuda et al., which is incorporated herein by reference, describes a capsule having an outer cylinder and a piston movable in the outer cylinder, the piston being activated by an externally given signal so as to discharge a medicine to the outside of the capsule or to suck a humor for a sampling purpose. The capsule has a remote-controllable means including a normally-opened lead switch which connects a power supply to an activating means in response to an externally given magnetic signal thereby initiating activation of the capsule.

U.S. Pat. No. 5,464,395 to Faxon et al., which is incorporated herein by reference, describes a catheter for delivering therapeutic and/or diagnostic agents directly into the tissue surrounding a bodily passageway. The catheter comprises at least one needle cannula able to be projected outboard of the catheter so as to deliver the desired agents to the tissue. The catheter also preferably includes one or more inflatable balloons.

U.S. Pat. No. 5,925,030 to Gross et al., which is incorporated herein by reference, describes an oral drug delivery device having a housing with walls of water permeable material, and having at least two chambers separated by a displaceable membrane. The first chamber receives a drug and has an orifice through which the drug is expelled under pressure. The second chamber contains at least one of two spaced apart electrodes forming part of an electrical circuit which is closed by the ingress of an aqueous ionic solution into the second chamber. When current flows through the circuit, gas is generated and acts on the displaceable membrane to compress the first chamber and expel the active ingredient through the orifice for progressive delivery to the GI tract.

U.S. Pat. No. 4,239,040 to Hosoya et al., which is incorporated herein by reference, describes a capsule for discharging drugs into a body or collecting samples from the body. The capsule comprises an external cylinder having slidably mounted therein an internal cylinder. The internal cylinder is retained by a meltable thread at one end of the external cylinder against the biasing force of a compression spring. Upon melting of the thread, the spring effects sliding of the internal cylinder to the other end of the external cylinder, and, during this sliding movement, a drug is pushed out of the external cylinder ahead of the moving internal cylinder or a body sample is withdrawn into the external cylinder behind the moving internal cylinder. An electric circuit including a tunable receiver responds to an externally-transmitted electric signal to energize a heater for melting the thread to thereby effect sliding movement of the internal cylinder at the desired time.

U.S. Pat. No. 4,425,117 to Hugemann et al., which is incorporated herein by reference, describes a capsule for the release of a substance at a defined or desired location in the alimentary tract. The capsule has a separating wall therein, which forms a first chamber and a second chamber, the first chamber having a hole in a wall thereof. A compression spring, in a compressed state, is affixed to a body located in the second chamber. A needle is mounted on the compression spring facing the separation wall. A resonant circuit in the second chamber is tuned to an electromagnetic field of high frequency. The resonant circuit has a coupling coil, positioned around the body, a capacitor, connected to the other end of the coil and extending away from the first chamber, and a resistance wire, attached to the coupling coil and the capacitor. A fuse wire is connected to the compression spring, extends through the longitudinal passageway of the body and is connected to the body end facing away from the first chamber. The fuse wire contacts the resistance wire. A balloon in the expanded state is positioned in the first chamber. When the device is subjected to an external electromagnetic field having the high frequency to which the resonant circuit is tuned, the fuse wire heats up and breaks. The compressed spring is released pushing the point of the needle through the separating wall and the balloon, which bursts releasing any substance contained in the first chamber.

U.S. Pat. No. 4,507,115 to Kambara et al., which is incorporated herein by reference, describes a capsule that comprises a capsule body having a chamber formed inside and a communicating path for communicating the chamber with outside, a movable member arranged in the chamber and movable between a liquid-receiving position at which the volume of said chamber is made largest and a liquid-pushing position at which the volume of said chamber is made smallest, and a coiled operating member made of shape memory alloy heated by ultrasonic wave to move the movable member to liquid-receiving and -pushing positions selectively.

U.S. Pat. No. 5,951,538 to Joshi et al., which is incorporated herein by reference, describes a controlled delivery device for holding and administering a biologically active agent. The device includes a housing having a first end portion, a second end portion, and a port associated with the housing. Enclosed within the housing is a displacing member, a chemical or electrochemical gas generating cell, and activation and control circuitry. The electrochemical or chemical cell generates gas within the housing, forcing the displacing member against the beneficial agents contained within the housing and forcing the beneficial agents through an outlet port and into a body cavity at a predetermined rate. An anchoring mechanism may be associated with the housing for securing the housing inside the body cavity.

U.S. Pat. Nos. 5,167,626 and 5,170,801 to Casper at al., which are incorporated herein by reference, describe a capsule for releasing a substance at a defined location in the GI tract. The body of the capsule defines one or more apertures in the circumferential wall thereof, and a sleeve valve rotatably positioned therein has one or more corresponding apertures in the circumferential wall thereof. The sleeve valve comprises a coil and electrically connected heatable resistor which are operatively associated with an actuator member formed of a shape memory alloy responsive to heat and which will move from a non-heated first shape to a heated second shape. Actuator stop means are provided in the capsule body for being engaged by the actuator member during movement from the non-heated first shape to the heated second shape so that the actuator member movement serves to rotate the sleeve valve to an open position.

PCT Publication WO 01/45552 to Houzego et al., which is incorporated herein by reference, describes a closure member for a substance reservoir of a site-specific drug delivery capsule (SSDC). The SSDC includes a retainer that provides a non-linear force resisting opening of the closure member. The non-linear force is described as ensuring that the closure member unseals the reservoir only when an opening force exceeds a maximal value of the resisting force, thereby preventing premature or accidental emptying of the reservoir. The preferred means of providing the resistive force is a rolling, elastomeric o-ring that additionally seals the closure member into an aperture.

U.S. Pat. No. 6,344,027 to Goll, which is incorporated herein by reference, describes techniques for delivering and injecting fluid into heart tissue utilizing high pressure injection to increase injectate (fluid) retention in the heart tissue. A catheter is described which includes a shaft having an infusion lumen extending therethrough, wherein the proximal end of the shaft connected to a pressurized fluid source capable of generating a transient pressure of more than 1000 psi. The distal end of the shaft includes a nozzle having an injection port in fluid communication with the infusion lumen such that fluid from the pressurized fluid source may be delivered to the heart tissue at a sufficiently high exit velocity to partially penetrate the heart tissue.

U.S. Pat. No. 6,369,039 to Palasis et al., which is incorporated herein by reference, describes a method for site-specifically delivering a therapeutic agent to a target location within a body cavity, vasculature or tissue. The method comprises: providing a medical device having a substantially saturated solution of therapeutic agent associated therewith; introducing the medical device into the body cavity, vasculature or tissue; releasing a volume of the solution of therapeutic agent from the medical device at the target location at a pressure of from about 0 to about 5 atmospheres for a time of up to about 5 minutes; and withdrawing the medical device from the body cavity, vasculature or tissue. The patent also describes a system for delivering a therapeutic agent to a body cavity, vasculature or tissue, comprising a medical device having a substantially saturated solution of the therapeutic agent associated therewith.

U.S. Pat. No. 5,964,726 to Korenstein et al., which is incorporated herein by reference, describes techniques for introducing molecules and macromolecules into a membrane vesicle, a cell, or a tissue by (a) applying a train of low unipolar or alternating voltage pulses to molecules/macromolecules and cells, (b) increasing the concentration of the molecules/macromolecules at the surface of the cells, leading to an increased interaction of the molecules/macromolecules with the membrane of the cell while also causing electrophoretic movement of charged proteins and lipids in the cell membrane, and (c) causing the destabilization of the cell membrane whereby the molecules/macromolecules penetrate into the cytosol via an endocytic process and via diffusion through structural defects in the membrane lipid bilayer.

PCT Publication WO 02/098501 to Keisari et al., which is incorporated herein by reference, describes a method for treating tumor tissue, including applying to cells of the tumor tissue electrical field pulses having a strength, a repetition frequency, and a pulse width selected capable of inducing endocytosis-mediated cell death, thereby treating the tumor tissue.

U.S. Pat. No. 3,659,600 to Merrill, which is incorporated herein by reference, describes an implantable capsule activated by magnetic force to release a drug. U.S. Pat. Nos. 3,485,235 to Felson, 3,315,660 to Abella, 3,118,439 to Perrenoud, and 3,057,344 to Abella et al., which are incorporated herein by reference, describe capsules for insertion into the GI tract for treatment and/or diagnostic purposes.

U.S. Pat. No. 6,572,740 to Rosenblum et al., which is incorporated herein by reference, describes electrolytic cells comprising (a) the electrolyte K₂HPO₄, or a less alkaline phosphate buffer solution, (b) electrodes having a modified composition, or (c) a combination of the electrolyte and a modified composition electrode. The K₂HPO₄ electrolyte, or less alkaline phosphate buffer solution, and modified electrodes can be used in liquid delivery devices which deliver a liquid agent at a constant rate or a controlled variable rate over a period of time.

An article by Lambert et al., entitled, “Autonomous telemetric capsule to explore the small bowel,” Med Biol Eng Comput 29(2):191-6 (1991), which is incorporated herein by reference, describes an intestinal telemetric capsule developed to study the small bowel in man. It consists of a cylinder (11 mm in diameter and 39 mm in length) containing a location detector, a radiotransmitter, a lithium battery and an interchangeable tip. After having been swallowed by the patient, the capsule passes through the whole gut and is recovered in the stool. During the transit through the small bowel, the information provided by the radiotransmitter allows continuous monitoring of the distance covered from the pylorus, as well as the direction and the velocity of progression. Moreover, according to the type of interchangeable tip, it is possible, by remote control, to sample 0.5 ml of intraluminal fluid for subsequent analysis or to release 1 ml of any liquid substance in a precisely-determined place for pharmacological studies.

The following articles, which are incorporated herein by reference, may be of interest:
Leonard M et al., “Iontophoresis-enhanced absorptive flux of polar molecules across intestinal tissue in vitro,” Pharm Res 17(4):476-8 (2000)
Ghartey-Tagoe E B et al., “Electroporation-mediated delivery of molecules to model intestinal epithelia,” Int J Pharm 270(1-2):127-38 (2004)
Hildebrand K R et al., “Intrinsic neuroregulation of ion transport in porcine distal jejunum,” J Pharmacol Exp Ther 255(1):285-92 (1990)
Neunlist M et al., “Human ENS regulates the intestinal epithelial barrier permeability and a tight junction-associated protein ZO-1 via VIPergic pathways,” Am J Physiol Gastrointest Liver Physiol 285(5) :G1028-36 (2003) (Epub July 24, 2003)

### SUMMARY OF THE INVENTION

In some embodiments of the present invention, an ingestible active drug-delivery system comprises electrical means to enhance the absorption of a drug provided to the gastrointestinal (GI) tract. For some applications, such means includes a device for performing electrotransport of the drug, in order to actively deliver the drug through the wall of the GI tract. Typically, the drug-delivery system comprises a pill-shaped and -sized capsule that comprises the delivery means, and holds the drug until it is released to the GI tract.

Typically, the active driving of the drug through the GI tract wall is accomplished by: (a) driving the drug through the wall by passage of the drug through tight junctions of the epithelial layer of the small intestine, and/or (b) driving the drug through the wall by penetrating the epithelial cells themselves. Typically, a therapeutically-significant portion of the drug is thereby passed into direct contact with the capillary supply of the GI tract, and therefrom into the systemic circulation. It is noted that this embodiment therefore typically allows entry into the bloodstream of drug molecules which would normally be largely excluded (e.g., due to size or chemical properties).

In some embodiments of the present invention, the drug-delivery system comprises an electrical signal generator and at least two electrodes, designed for facilitating electrotransport. For some applications, electrotransport is facilitated by applying a “low intensity time-varying” (LITV) signal, which is to be understood in the present application, including the claims, as including an electrical signal that is selected from the list consisting of:
a signal that creates a field that is less than about 5 Volts/cm and varies at a rate greater than about 1 Hz;
a signal capable of opening tight junctions of the epithelial layer of the GI tract to an extent sufficient to allow at least a 100% increase in the passage of a drug therethrough (relative to an extent of passage of the drug therethrough in the absence of the LITV signal); and
a signal insufficient to cause electroporation of cells of the epithelial layer of the GI tract.

Alternatively or additionally, the electrotransport includes any one of, or a combination of, iontophoresis, electroosmosis, and electrophoresis, which enhance diffusion processes through the epithelial cells, and/or electroporation. Electroporation is to be understood in the present application, including the claims (notwithstanding any other definitions which may be found in any of the patents, patent applications, or articles incorporated herein by reference), as electrotransport, which, typically using high voltage, creates transient permeable structures or micropores in the epithelial cell membranes, enabling passage of large molecules through the epithelium.

In some embodiments of the present invention, parameters for effecting the electrotransport are selected based at least in part on the particular properties of the drug. Drugs comprising larger molecules typically require stronger stimulation. Alternatively or additionally, the parameters are selected based at least in part on the portion of the GI tract to which the drug is to be delivered. Typically, parameters are selected that apply the lowest amount of energy sufficient to achieve drug passage through the GI tract wall.

In some embodiments of the present invention, the drug-delivery system comprises a mechanism that is operative to be responsive to its environment, such as, for example, a pH-sensitive coating. The coating is typically configured, using techniques known in the art, to dissolve upon entering a small intestine of a patient. In accordance with other embodiments of the present invention, the environmentally-responsive mechanism comprises, for example, a sensor (such as an electronic sensor, and/or a temperature sensor or a pH sensor), a timer, a transmitter/receiver, or a camera.

In some embodiments of the present invention, the dissolving of the coating triggers activation of the driving means, which, in turn, actively drives drug through the wall of the GI tract wall. For some applications, the coating is configured to dissolve in a pH range typical of the small intestine.

In some embodiments of the present invention, the coating is applied at a first thickness over a first portion of the capsule, and at a second thickness over a second portion of the capsule. Alternatively or additionally, different types of coatings are applied to different portions of the capsule, e.g., in order to provide for the respective portions of the capsule to be exposed to the small intestine at different times.

In some embodiments of the present invention, the functionality for activating the driving mechanism, described hereinabove as being provided by a coating, is supplemented or replaced by other activating functionalities. For some applications, the capsule comprises a bio-sensor that detects a biological or physiological parameter, and activates the driving mechanism responsive thereto. As appropriate, the bio-sensor may comprise one or more of the following: an enzymatic sensor, a temperature sensor, a pH sensor, or a timer (the timer typically comprising chemicals that react in a known manner to activate the driving mechanism at a predetermined time following an event such as the patient squeezing the capsule or the patient ingesting the capsule). Alternatively or additionally, the capsule comprises a camera, which records an image of the GI tract for on-board analysis and, if appropriate, activation of the driving mechanism in response to the image.

For some applications, the capsule comprises a transmit/receive unit, adapted to transmit a signal responsive to an image recorded by the camera and/or responsive to a reading by the bio-sensor. The transmitted data are typically analyzed in real-time, and a decision is made (e.g., by a physician or by a computer external to the patient) whether and when to administer drug.

In some embodiments of the present invention, an ingestible, electrically-assisted drug-delivery facilitation system comprises electrical means to enhance the absorption of a drug contained in a commercially-available drug pill that is ingested by a patient in conjunction with ingesting the drug-delivery system, e.g., before, simultaneously with, or after ingesting the system. The system thus serves to enhance absorption of the drug released from the drug pill in the GI tract. In these embodiments, the drug-delivery system does not contain the drug, and is not assembled in an integral unit with the drug.

In some embodiments of the present invention, an ingestible, electrically-assisted drug-delivery facilitation system comprises electrical means to enhance the absorption of a drug contained in a commercially-available drug pill coupled to the system. The pill may be coupled to the system by a manufacturer, the patient, or a healthcare worker, depending, for example, on medical, safety, commercial, or other considerations.

There is therefore provided, in accordance with an embodiment of the present invention, apparatus for drug administration, including an ingestible capsule, which includes:
a drug, stored by the capsule;
an environmentally-sensitive mechanism, adapted to change a state thereof responsively to a disposition of the capsule within a gastrointestinal (GI) tract of a subject;
first and second electrodes; and
a control component, adapted to facilitate passage of the drug, in response to a change of state of the environmentally-sensitive mechanism, through an epithelial layer of the GI tract by driving the first and second electrodes to apply a series of pulses at a current of less than about 5 mA, at a frequency of between about 12 Hz and about 24 Hz, and with a pulse duration of between about 0.5 milliseconds and about 3 milliseconds.

For some applications, the pulses include monophasic rectangular pulses, and the control component is adapted to drive the first and second electrodes to apply the series of monophasic rectangular pulses.

For some applications, the first and second electrodes include stainless steel.

For some applications, the environmentally-sensitive mechanism includes a sensor adapted to sense an indication of a distance traveled by the capsule in the GI tract, and the environmentally-sensitive mechanism is adapted to undergo the change of state responsive to the distance. Alternatively or additionally, the environmentally-sensitive mechanism includes a camera, adapted to image the GI tract, and the control component is adapted to drive the first and second electrodes to apply the series of pulses in response to an image acquired by the camera.

For some applications, the disposition of the capsule includes a temperature in a vicinity of the capsule, the environmentally-sensitive mechanism includes a temperature sensor, and the control component is adapted to drive the first and second electrodes to apply the series of pulses in response to the temperature sensed by the temperature sensor. Alternatively or additionally, the disposition of the capsule includes a pH in a vicinity of the capsule, the environmentally-sensitive mechanism includes a pH sensor, and the control component is adapted to drive the first and second electrodes to apply the series of pulses in response to the pH sensed by the pH sensor.

For some applications, the environmentally-sensitive mechanism includes a sensor, adapted to sense a characteristic of the GI tract, and the control component is adapted to drive the first and second electrodes to apply the series of pulses in response to the sensed characteristic.

For some applications, the control component is adapted to drive the first and second electrodes to apply the series of pulses, and to drive an iontophoretic current between the first and second electrodes.

For some applications, the control component is adapted to configure the series of pulses using parameters selected at least in part responsively to the disposition of the capsule within the GI tract. Alternatively or additionally, the control component is adapted to configure the series of pulses using parameters selected at least in part responsively to a property of the drug.

For some applications, the capsule includes a central portion, intermediate the first and second electrodes, a shape of the central portion being such as to reduce current flow within a lumen of the GI tract. For some applications, the capsule includes a central portion, intermediate the first and second electrodes, the central portion having a diameter that is such as to bring the central portion in contact with the epithelial layer of the GI tract, whereby to reduce current flow within a lumen of the GI tract. For some applications, the capsule includes a self-expansible central portion, intermediate the first and second electrodes, the central portion adapted to expand, in response to being in the GI tract, to have a diameter that is such as to bring the central portion in contact with the epithelial layer of the GI tract, whereby to reduce current flow within a lumen of the GI tract. For some applications, the capsule includes a central portion, intermediate the first and second electrodes, an outer surface of the central portion including a hydrophobic material. For some applications, the capsule includes a central portion, intermediate the first and second electrodes, an outer surface of the central portion including a lipophilic material.

For some applications, the environmentally-sensitive mechanism is essentially entirely biodegradable. For some applications, the first and second electrodes and the control component are essentially entirely biodegradable.

For some applications, at least 80% of the mass of the capsule is biodegradable. For some applications, at least 95% of the mass of the capsule is biodegradable. For some applications, essentially the entire capsule is biodegradable.

For some applications, the environmentally-sensitive mechanism includes a coating on a surface of the capsule. For some applications, the coating includes a pH-sensitive coating.

In an embodiment, the control component is adapted to apply the series of pulses at a current of between about 2 mA and about 4 mA. For some applications, the control component is adapted to drive the first and second electrodes to apply the series of pulses at a current of about 3 mA.

In an embodiment, the control component is adapted to drive the first and second electrodes to apply the series of pulses at a frequency of between about 16 Hz and about 20 Hz. For some applications, the control component is adapted to drive the first and second electrodes to apply the series of pulses at a frequency of about 18 Hz.

In an embodiment, the control component is adapted to drive the first and second electrodes to apply the series of pulses with a pulse duration of between about 0.5 milliseconds and about 1.5 milliseconds. For some applications, the control component is adapted to drive the first and second electrodes to apply the series of pulses with a pulse duration of about 1 millisecond.

In an embodiment, the control component is adapted to drive the first and second electrodes to apply the series of pulses for a period of between about 1 and about 360 minutes. For some applications, the control component is adapted to drive the first and second electrodes to apply the series of pulses for a period of between about 60 and about 240 minutes.

There is also provided, in accordance with an embodiment of the present invention, apparatus for administration of a drug, including an ingestible capsule adapted to store the drug, the capsule including:
an environmentally-sensitive mechanism, adapted to change a state thereof responsively to a disposition of the capsule within a gastrointestinal (GI) tract of a subject;
first and second electrodes; and
a control component, adapted to facilitate passage of the drug, in response to a change of state of the environmentally-sensitive mechanism, through an epithelial layer of the GI tract by driving the first and second electrodes to apply a series of pulses at a current of less than about 5 mA, at a frequency of between about 12 Hz and about 24 Hz, and with a pulse duration of between about 0.5 milliseconds and about 3 milliseconds.

In an embodiment, the control component is adapted to apply the series of pulses at a current of between about 2 mA and about 4 mA. For some applications, the control component is adapted to drive the first and second electrodes to apply the series of pulses at a current of about 3 mA.

In an embodiment, the control component is adapted to drive the first and second electrodes to apply the series of pulses at a frequency of between about 16 Hz and about 20 Hz. For some applications, the control component is adapted to drive the first and second electrodes to apply the series of pulses at a frequency of about 18 Hz.

In an embodiment, the control component is adapted to drive the first and second electrodes to apply the series of pulses with a pulse duration of between about 0.5 milliseconds and about 1.5 milliseconds. For some applications, the control component is adapted to drive the first and second electrodes to apply the series of pulses with a pulse duration of about 1 millisecond.

In an embodiment, the control component is adapted to drive the first and second electrodes to apply the series of pulses for a period of between about 1 and about 360 minutes. For some applications, the control component is adapted to drive the first and second electrodes to apply the series of pulses for a period of between about 60 and about 240 minutes.

There is further provided, in accordance with an embodiment of the present invention, apparatus for facilitating administration of a drug contained in a pill, the apparatus including an ingestible housing, which is not adapted to contain the drug or to be assembled in an integral unit with the drug, the housing including:
an ingestible environmentally-sensitive mechanism, adapted to change a state thereof responsive to a disposition thereof within a gastrointestinal (GI) tract of a subject;
first and second electrodes; and
a control component, adapted to facilitate passage of the drug, in response to a change of state of the environmentally-sensitive mechanism, through an epithelial layer of the GI tract by driving the first and second electrodes to apply a series of pulses at a current of less than about 5 mA, at a frequency of between about 12 Hz and about 24 Hz, and with a pulse duration of between about 0.5 milliseconds and about 3 milliseconds.

For some applications, the environmentally-sensitive mechanism includes a sensor adapted to sense an indication of a distance traveled by the housing in the GI tract, and the environmentally-sensitive mechanism is adapted to undergo the change of state responsive to the distance.

For some applications, the environmentally-sensitive mechanism includes a camera, adapted to image the GI tract, and the control component is adapted to drive the first and second electrodes to apply the series of pulses in response to an image acquired by the camera.

For some applications, the disposition of the environmentally-sensitive mechanism includes a temperature in a vicinity of the environmentally-sensitive mechanism, the environmentally-sensitive mechanism includes a temperature sensor, and the control component is adapted to drive the first and second electrodes to apply the series of pulses in response to the temperature sensed by the temperature sensor.

For some applications, the disposition of the environmentally-sensitive mechanism includes a pH in a vicinity of the environmentally-sensitive mechanism, the environmentally-sensitive mechanism includes a pH sensor, and the control component is adapted to drive the first and second electrodes to apply the series of pulses in response to the pH sensed by the pH sensor.

For some applications, the environmentally-sensitive mechanism includes a sensor, adapted to sense a characteristic of the GI tract, and the control component is adapted to drive the first and second electrodes to apply the series of pulses in response to the sensed characteristic.

For some applications, the environmentally-sensitive mechanism is adapted to undergo the change of state generally at an expected time of release of the drug from the drug pill.

For some applications, the environmentally-sensitive mechanism includes a coating on a surface of the housing. For some applications, the coating includes a pH-sensitive coating.

In an embodiment, the control component is adapted to apply the series of pulses at a current of between about 2 mA and about 4 mA. For some applications, the control component is adapted to drive the first and second electrodes to apply the series of pulses at a current of about 3 mA.

In an embodiment, the control component is adapted to drive the first and second electrodes to apply the series of pulses at a frequency of between about 16 Hz and about 20 Hz. For some applications, the control component is adapted to drive the first and second electrodes to apply the series of pulses at a frequency of about 18 Hz.

In an embodiment, the control component is adapted to drive the first and second electrodes to apply the series of pulses with a pulse duration of between about 0.5 milliseconds and about 1.5 milliseconds. For some applications, the control component is adapted to drive the first and second electrodes to apply the series of pulses with a pulse duration of about 1 millisecond.

In an embodiment, the control component is adapted to drive the first and second electrodes to apply the series of pulses for a period of between about 1 and about 360 minutes. For some applications, the control component is adapted to drive the first and second electrodes to apply the series of pulses for a period of between about 60 and about 240 minutes.

There is additionally provided, in accordance with an embodiment of the present invention, apparatus for use with a drug pill, the apparatus including:
a coupling mechanism, adapted to couple the drug pill to the apparatus;
first and second electrodes; and
a control component, adapted to facilitate passage of a drug contained in the drug pill through an epithelial layer of a gastrointestinal (GI) tract of a subject by driving the first and second electrodes to apply a series of pulses at a current of less than about 5 mA, at a frequency of between about 12 Hz and about 24 Hz, and with a pulse duration of between about 0.5 milliseconds and about 3 milliseconds.

For some applications, the drug pill includes a commercially-available drug pill, and the coupling mechanism is adapted to couple the commercially-available drug pill to the apparatus. For some applications, the coupling mechanism includes an adhesive.

For some applications, the coupling mechanism includes at least one of the electrodes. For some applications, the at least one of the electrodes is configured to surround a portion of the drug pill once the drug pill has been coupled to the apparatus.

In an embodiment, the control component is adapted to apply the series of pulses at a current of between about 2 mA and about 4 mA. For some applications, the control component is adapted to drive the first and second electrodes to apply the series of pulses at a current of about 3 mA.

In an embodiment, the control component is adapted to drive the first and second electrodes to apply the series of pulses at a frequency of between about 16 Hz and about 20 Hz. For some applications, the control component is adapted to drive the first and second electrodes to apply the series of pulses at a frequency of about 18 Hz.

In an embodiment, the control component is adapted to drive the first and second electrodes to apply the series of pulses with a pulse duration of between about 0.5 milliseconds and about 1.5 milliseconds. For some applications, the control component is adapted to drive the first and second electrodes to apply the series of pulses with a pulse duration of about 1 millisecond.

In an embodiment, the control component is adapted to drive the first and second electrodes to apply the series of pulses for a period of between about 1 and about 360 minutes. For some applications, the control component is adapted to drive the first and second electrodes to apply the series of pulses for a period of between about 60 and about 240 minutes.

There is yet additionally provided, in accordance with an embodiment of the present invention, apparatus for facilitating administration of a drug to a subject, the apparatus including:
a sensor unit, which includes:
   a sensor, adapted to detect an indication of a concentration of a substance in a blood circulation of the subject; and
   a wireless transmitter, adapted to wirelessly transmit the indication; and
an ingestible capsule, which includes:
   a wireless receiver, adapted to receive the indication;
   first and second electrodes; and
   a control component, adapted to facilitate passage of the drug through an epithelial layer of a gastrointestinal (GI) tract of the subject by driving the first and second electrodes to apply a series of pulses at a current of less than about 5 mA, at a frequency of between about 12 Hz and about 24 Hz, and with a pulse duration of between about 0.5 milliseconds and about 3 milliseconds.

For some applications, the substance includes the drug, and the sensor is adapted to detect the indication of the concentration of the drug in the blood circulation.

For some applications, the substance includes a calibrating substance, the sensor is adapted to detect the indication of the concentration of the calibrating substance in the blood circulation, and the control component is adapted to facilitate the passage of the calibrating substance and the drug through the epithelial layer of the GI tract, responsively to the received indication.

For some applications, the sensor includes a noninvasive external sensor. Alternatively, the sensor includes an invasive sensor.

For some applications, the ingestible capsule is adapted to store the drug. Alternatively, the ingestible capsule is not adapted to contain the drug or to be assembled in an integral unit with the drug.

For some applications, the drug is contained in a drug pill, and the ingestible capsule includes a coupling mechanism, adapted to couple the drug pill to the ingestible capsule.

For some applications, the ingestible capsule includes an environmentally-sensitive mechanism, adapted to change a state thereof responsively to a disposition of the capsule within the GI tract, and the control component is adapted to facilitate the passage of the drug through the epithelial layer in response to a change of state of the environmentally-sensitive mechanism.

For some applications, the indication includes respective first and second indications, sensed at respective first and second times, the wireless transmitter is adapted to transmit the first indication subsequent to the first time, and to transmit the second indication subsequent to the second time, and the control component is adapted to drive the first and second electrodes to apply first and second series of pulses, responsive to the first and second indications. For some applications, the sensor unit is adapted to space the first and second times by at least 10 minutes. For some applications, the control component is adapted to regulate a parameter of at least one of the series of pulses, responsive to at least one of the indications.

For some applications, the ingestible capsule includes a capsule wireless transmitter, the sensor unit includes a sensor unit wireless receiver, and the ingestible capsule is adapted to wirelessly notify the sensor unit of a property of the capsule, via the capsule wireless transmitter and the sensor unit wireless receiver. For some applications, the property is selected from the list consisting of: a location of the capsule, a status of the control component, a pH level of the GI tract, and a temperature of the GI tract, and the capsule is adapted to wirelessly notify the sensor of the selected property.

For some applications, the substance includes a chemical, the blood concentration of which is affected by a blood concentration of the drug, and the sensor is adapted to detect the indication of the concentration of the chemical in the blood circulation. For some applications, the chemical is selected from the list consisting of: glucose, growth hormone, and hemoglobin-bound oxygen, and the sensor is adapted to detect the indication of the concentration of the selected chemical in the blood circulation.

In an embodiment, the control component is adapted to apply the series of pulses at a current of between about 2 mA and about 4 mA. For some applications, the control component is adapted to drive the first and second electrodes to apply the series of pulses at a current of about 3 mA.

In an embodiment, the control component is adapted to drive the first and second electrodes to apply the series of pulses at a frequency of between about 16 Hz and about 20 Hz. For some applications, the control component is adapted to drive the first and second electrodes to apply the series of pulses at a frequency of about 18 Hz.

In an embodiment, the control component is adapted to drive the first and second electrodes to apply the series of pulses with a pulse duration of between about 0.5 milliseconds and about 1.5 milliseconds. For some applications, the control component is adapted to drive the first and second electrodes to apply the series of pulses with a pulse duration of about 1 millisecond.

In an embodiment, the control component is adapted to drive the first and second electrodes to apply the series of pulses for a period of between about 1 and about 360 minutes. For some applications, the control component is adapted to drive the first and second electrodes to apply the series of pulses for a period of between about 60 and about 240 minutes.

There is still additionally provided, in accordance with an embodiment of the present invention, apparatus for facilitating administration of a drug to a subject, the apparatus including:
a sensor unit, which includes:
   a sensor, adapted to detect an indication of a physiological parameter of the subject; and
   a wireless transmitter, adapted to wirelessly transmit the indication; and
an ingestible capsule, which includes:
   a wireless receiver, adapted to receive the indication;
   first and second electrodes; and
   a control component, adapted to facilitate passage of the drug through an epithelial layer of a gastrointestinal (GI) tract of the subject by driving the first and second electrodes to apply a series of pulses at a current of less than about 5 mA, at a frequency of between about 12 Hz and about 24 Hz, and with a pulse duration of between about 0.5 milliseconds and about 3 milliseconds.

For some applications, the indication includes an indication of blood pressure of the subject, and the sensor is adapted to sense the indication of blood pressure. Alternatively or additionally, the indication includes an indication of a heart-related parameter of the subject, and the sensor is adapted to sense the indication of the heart-related parameter. Further alternatively or additionally, the indication includes an indication of a level of activity of the subject, and the sensor is adapted to sense the indication of the level of activity.

For some applications, the indication includes an indication of a temperature of the subject, and the sensor is adapted to sense the indication of the temperature. Alternatively or additionally, the indication includes an indication of a circadian cycle of the subject, and the sensor includes clock circuitry adapted to sense the indication of the circadian cycle.

In an embodiment, the control component is adapted to apply the series of pulses at a current of between about 2 mA and about 4 mA. For some applications, the control component is adapted to drive the first and second electrodes to apply the series of pulses at a current of about 3 mA.

In an embodiment, the control component is adapted to drive the first and second electrodes to apply the series of pulses at a frequency of between about 16 Hz and about 20 Hz. For some applications, the control component is adapted to drive the first and second electrodes to apply the series of pulses at a frequency of about 18 Hz.

In an embodiment, the control component is adapted to drive the first and second electrodes to apply the series of pulses with a pulse duration of between about 0.5 milliseconds and about 1.5 milliseconds. For some applications, the control component is adapted to drive the first and second electrodes to apply the series of pulses with a pulse duration of about 1 millisecond.

In an embodiment, the control component is adapted to drive the first and second electrodes to apply the series of pulses for a period of between about 1 and about 360 minutes. For some applications, the control component is adapted to drive the first and second electrodes to apply the series of pulses for a period of between about 60 and about 240 minutes.

There is still further provided, in accordance with an embodiment of the present invention, apparatus for facilitating administration of a drug to a subject, the apparatus including:
first and second electrodes; and
a control component, adapted to facilitate passage of the drug through an epithelial layer of a gastrointestinal (GI) tract of the subject by driving the first and second electrodes to apply a series of pulses at a current of less than about 5 mA, at a frequency of between about 12 Hz and about 24 Hz, and with a pulse duration of between about 0.5 milliseconds and about 3 milliseconds.

In an embodiment, the control component is adapted to apply the series of pulses at a current of between about 2 mA and about 4 mA. For some applications, the control component is adapted to drive the first and second electrodes to apply the series of pulses at a current of about 3 mA.

In an embodiment, the control component is adapted to drive the first and second electrodes to apply the series of pulses at a frequency of between about 16 Hz and about 20 Hz. For some applications, the control component is adapted to drive the first and second electrodes to apply the series of pulses at a frequency of about 18 Hz.

In an embodiment, the control component is adapted to drive the first and second electrodes to apply the series of pulses with a pulse duration of between about 0.5 milliseconds and about 1.5 milliseconds. For some applications, the control component is adapted to drive the first and second electrodes to apply the series of pulses with a pulse duration of about 1 millisecond.

In an embodiment, the control component is adapted to drive the first and second electrodes to apply the series of pulses for a period of between about 1 and about 360 minutes. For some applications, the control component is adapted to drive the first and second electrodes to apply the series of pulses for a period of between about 60 and about 240 minutes.

There is also provided, in accordance with an embodiment of the present invention, a method for administration of a drug, including:
administering to a subject an ingestible capsule that includes the drug;
detecting a disposition of the capsule within a gastrointestinal (GI) tract of the subject; and
in response to detecting the disposition, facilitating, by the capsule, passage of the drug through an epithelial layer of the GI tract, by applying a series of pulses at a current of less than about 5 mA, at a frequency of between about 12 Hz and about 24 Hz, and with a pulse duration of between about 0.5 milliseconds and about 3 milliseconds.

There is further provided, in accordance with an embodiment of the present invention, a method for administration of a drug contained in a pill, including:
orally administering the pill to a subject;
orally administering to the subject an ingestible capsule that does not include the drug;
detecting a target location of the capsule within a gastrointestinal (GI) tract of the subject; and
in response to detecting the target location, facilitating, by the capsule, passage of the drug through an epithelial layer of the GI tract, by applying a series of pulses at a current of less than about 5 mA, at a frequency of between about 12 Hz and about 24 Hz, and with a pulse duration of between about 0.5 milliseconds and about 3 milliseconds.

There is still further provided, in accordance with an embodiment of the present invention, a method for administration of a drug, including:
coupling, to an ingestible capsule, a drug pill containing the drug;
administering the capsule to a subject;
detecting a target location of the capsule within a gastrointestinal (GI) tract of the subject; and
in response to detecting the target location, facilitating, by the capsule, passage of the drug through an epithelial layer of the GI tract, by applying a series of pulses at a current of less than about 5 mA, at a frequency of between about 12 Hz and about 24 Hz, and with a pulse duration of between about 0.5 milliseconds and about 3 milliseconds.

There is additionally provided, in accordance with an embodiment of the present invention, a method for facilitating administration of a drug to a subject, the method including:
administering an ingestible capsule to the subject;
detecting an indication of a concentration of a substance in a blood circulation of the subject;
wirelessly transmitting the indication;
receiving the indication at the ingestible capsule; and
responsively to the received indication, facilitating, by the capsule, passage of the drug through an epithelial layer of a gastrointestinal (GI) tract of the subject, by applying a series of pulses at a current of less than about 5 mA, at a frequency of between about 12 Hz and about 24 Hz, and with a pulse duration of between about 0.5 milliseconds and about 3 milliseconds.

There is yet additionally provided, in accordance with an embodiment of the present invention, a method for facilitating administration of a drug to a subject, the method including:
administering an ingestible capsule to the subject;
detecting an indication of a physiological parameter of the subject;
wirelessly transmitting the indication;
receiving the indication at the ingestible capsule; and
responsively to the received indication, facilitating, by the capsule, passage of the drug through an epithelial layer of a gastrointestinal (GI) tract of the subject, by applying a series of pulses at a current of less than about 5 mA, at a frequency of between about 12 Hz and about 24 Hz, and with a pulse duration of between about 0.5 milliseconds and about 3 milliseconds.

For some applications, the indication includes an indication of a circadian cycle of the subject, and detecting the indication includes detecting the indication of the circadian cycle. For some applications, the drug includes an antithrombotic drug, and facilitating the passage of the drug includes facilitating the passage of the antithrombotic drug through the epithelial layer.

For some applications, the indication includes an indication of a temperature of the subject, and detecting the indication includes detecting the indication of the temperature. For some applications, the drug includes an antibiotic, and facilitating the passage of the drug includes facilitating the passage of the antibiotic through the epithelial layer.

There is also provided, in accordance with an embodiment of the present invention, a method for administration of a drug, including:
administering the drug to a gastrointestinal (GI) tract of a subject; and
facilitating passage of the drug through an epithelial layer of the GI tract by applying a series of pulses at a current of less than about 5 mA, at a frequency of between about 12 Hz and about 24 Hz, and with a pulse duration of between about 0.5 milliseconds and about 3 milliseconds.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of the intestinal wall;

FIG. 2 is a schematic illustration of a device for electrically-assisted drug delivery, in accordance with some embodiments of the present invention;

FIGS. 3A and 3B are schematic illustrations of ingestible, electrically-assisted drug-delivery systems, in accordance with embodiments of the present invention;

FIG. 4 is a schematic illustration of an ingestible, electrically-assisted drug-delivery system, having a plurality of electrodes, in accordance with an embodiment of the present invention;

FIG. 5 is a schematic illustration of another ingestible, electrically-assisted drug-delivery system, having a plurality of electrodes, in accordance with an embodiment of the present invention;

FIGS. 6A and 6B are schematic illustrations of an ingestible, electrically-assisted drug-delivery system, having self-expansible portions, in accordance with embodiment of the present invention;

FIG. 7 is a schematic illustration of an ingestible, electrically-assisted drug-delivery system, having a plurality of electrodes, in accordance with an embodiment of the present invention;

FIG. 8 is a schematic illustration of an ingestible, electrically-assisted drug-delivery system, having a plurality of electrodes and self-expansible portions, in accordance with an embodiment of the present invention;

FIG. 9 is a schematic illustration of another ingestible, electrically-assisted drug-delivery system, having a plurality of electrodes and self-expansible portions, in accordance with an embodiment of the present invention;

FIG. 10 is a schematic illustration of an ingestible, electrically-assisted drug-delivery system, having a plurality of electrodes and self-expansible portions, when in the gastrointestinal tract, in accordance with an embodiment of the present invention;

FIGS. 11A-11D are schematic illustrations of an ingestible, electrically-assisted drug-delivery system, wherein the drug-dispensing cavities are formed as self-expansible portions, in accordance with embodiments of the present invention;

FIG. 12 is a schematic illustration of an ingestible, electrically-assisted drug-delivery system, having a drug cavity with a biodegradable cap, in accordance with an embodiment of the present invention;

FIG. 13 is a schematic illustration of an ingestible, electrically-assisted drug-delivery system, wherein the drug is pressed into an integrated tablet with the system, in accordance with an embodiment of the present invention;

FIGS. 14A and 14B are schematic illustrations of an ingestible, electrically-assisted drug-delivery system, adapted to form an osmosis pump in the gastrointestinal tract, in accordance with embodiments of the present invention;

FIG. 15 is a schematic illustration of an ingestible, electrically-assisted drug-delivery system, having a pH-dependent controlled drug release, in accordance with an embodiment of the present invention;

FIG. 16 is a schematic illustration of an ingestible, electrically-assisted drug-delivery system, having an electronically activated, pH-dependent controlled drug release, in accordance with an embodiment of the present invention;

FIG. 17 is a schematic illustration of an ingestible, electrically-assisted drug-delivery system, adapted for sonophoresis, in accordance with an embodiment of the present invention;

FIG. 18 is a schematic illustration of an ingestible, electrically-assisted drug-delivery system, adapted for ablation, in accordance with an embodiment of the present invention;

FIG. 19 is a schematic illustration of an ingestible, electrically-assisted drug-delivery system, adapted for telemetry communication, in accordance with an embodiment of the present invention;

FIG. 20 is a schematic illustration of an ingestible, electrically-assisted drug-delivery system, adapted to make a galvanic cell with the body, in accordance with an embodiment of the present invention;

FIG. 21 is a schematic illustration of an ingestible, electrically-assisted drug-delivery facilitation system, in accordance with an embodiment of the present invention;

FIG. 22 is a schematic illustration of another ingestible, electrically-assisted drug-delivery system, in accordance with an embodiment of the present invention;

FIG. 23 is a schematic illustration of a coupling mechanism, in accordance with an embodiment of the present invention;

FIG. 24 is a graph showing in vitro experimental results measured in accordance with an embodiment of the present invention;

FIG. 25 is a schematic illustration of a closed-loop active drug-delivery system, in accordance with an embodiment of the present invention;

FIG. 26 is a schematic cross-sectional illustration of an experimental diffusion chamber, in accordance with an embodiment of the present invention; and

FIGS. 27-36 are graphs showing in vitro experimental results generated in accordance with respective embodiments of the present invention.

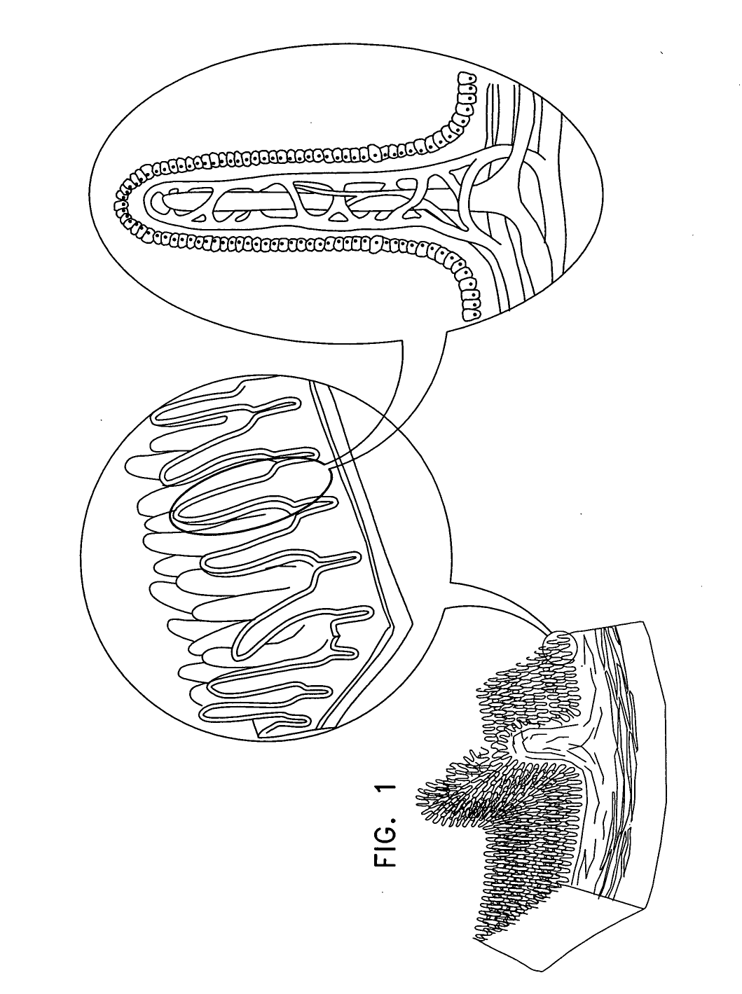

### DETAILED DESCRIPTION OF EMBODIMENTS

Some embodiments of the present invention comprise a typically ingestible, electrically-assisted, drug-delivery system. Specifically, these embodiments of the present invention act as a medication carrier, which utilizes electrically-induced means to enhance the absorption of the medication through the gastrointestinal (GI) tract walls.

The principles and operation of the typically ingestible, electrically-assisted, drug-delivery system, according to these embodiments of the present invention, may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

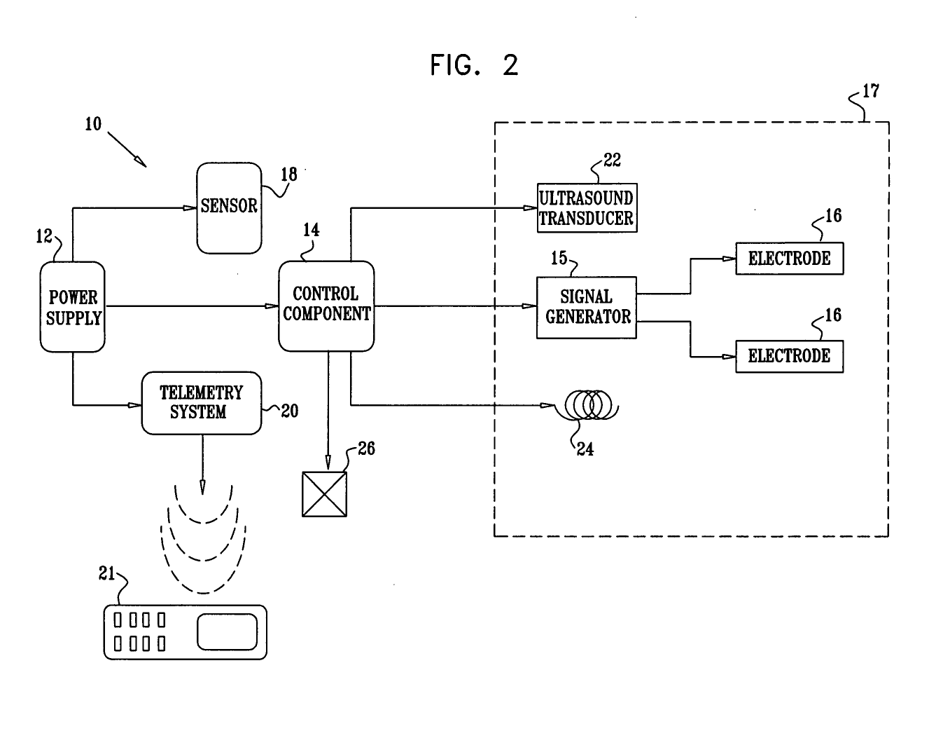

Referring now to the drawings, FIG. 2 is a schematic diagram of an electrically-assisted, drug-delivery device 10, in accordance with some embodiments of the present invention. Device 10 is biologically inert and biologically compatible, and is typically adapted for ingestion. Device 10 comprises a power supply 12, a control component 14 in power communication with power supply 12, and at least one apparatus 17 for electrically-assisted drug transport, which is in signal communication with control component 14 and in power communication with power supply 12. Control component 14 may be dedicated circuitry, a controller, or a microcomputer, as known in the art.

For some applications, apparatus 17 comprises an electrical signal generator 15 and at least two electrodes 16, designed for electrotransport. Alternatively, four or more electrodes 16 may be provided. Apparatus 17 may be designed, for example, as an electrotransport device, as described in any one, or a combination of, U.S. Pat. No. 5,674,196, to Donaldson et al., U.S. Pat. No. 5,961,482 to Chien et al., U.S. Pat. No. 5,983,131 to Weaver et al., U.S. Pat. No. 5,983,134 to Ostrow, and U.S. Pat. No. 6,477,410 to Henley et al., all of which are incorporated herein by reference. For some applications, electrodes 16 comprise stainless steel type 316S leads. Alternatively, the electrodes comprise other materials. For some applications, electrodes 16 have a surface area of between about 1 and about 100 mm², such as between about 10 and about 50 mm², e.g., 36 mm² or 42 mm².

Additionally or alternatively, apparatus 17 is designed for performing sonophoresis, or for performing a combination of sonophoresis and electrotransport, and comprises at least one ultrasound transducer 22. Apparatus 17 may be designed, for example, as a sonophoresis device, as described in any one, or a combination of, U.S. Pat. Nos. 6,002,961, 6,018,678, and 6,002,961 to Mitragotri et al., U.S. Pat. Nos. 6,190,315 and 6,041,253 to Kost et al., U.S. Pat. No. 5,947,921 to Johnson et al., and U.S. Pat. Nos. 6,491,657 and 6,234,990 to Rowe et al., all of which are incorporated herein by reference.

Additionally or alternatively, apparatus 17 is designed for performing ablation, or for performing a combination of ablation and electrotransport, ablation and sonophoresis, or ablation, electrotransport, and sonophoresis, and comprises at least one ablation apparatus 24. The ablation process may be, for example, any one of, or a combination of, laser ablation, cryogenic ablation, thermal ablation, microwave ablation, radiofrequency (RF) ablation, electrical ablation, and liquid jet ablation. Apparatus 17 may be designed, for example, as an ablation device, as described in any one, or a combination of, U.S. Pat. No. 6,471,696, to Berube et al. (which describes a microwave ablation catheter that may be used as a drug delivery device), U.S. Pat. No. 6,443,945 to Marchitto et al. (which describes a devices for pharmaceutical delivery using laser ablation), U.S. Pat. No. 4,869,248 to Narula (which describes a catheter for performing localized thermal ablation for drug administration), and U.S. Pat. Nos. 6,148,232 and 5,983,135 to Avrahami (which describe drug delivery systems using electrical ablation). All of these patents are incorporated herein by reference.

In accordance with some embodiments of the present invention, device 10 further comprises at least one sensor 18. Sensor 18 may be, for example, a physical sensor, such as a temperature sensor or a pressure sensor. Alternatively, sensor 18 may be a chemical sensor, such as a pH sensor or a drug-concentration sensor. Alternatively, sensor 18 may be a biological sensor, such as a glucose sensor or a bacterial-count sensor. For some applications, more than one sensor 18 is used. These may be of the same type or of different types.

In accordance with some embodiments of the present invention, device 10 further comprises a telemetry system 20, operative, for example, by RF, infrared radiation, or by ultrasound, for providing communication with an extracorporeal station 21, for example, a remote control. Alternatively or additionally, extracorporeal station 21 comprises a computer system. Alternatively or additionally, telemetry system 20 comprises a power transducer (such as a coil or a piezoelectric transducer), as is known in the art, adapted to receive electromagnetic radiation or ultrasonic energy, as appropriate, transmitted by extracorporeal station 21, and to transduce the radiation into a current for powering the operation of drug-delivery device 10. As appropriate, the power transducer may replace power supply 12, or supplement its operation.

In accordance with some embodiments of the present invention, device 10 further comprises at least one electronic valve 26 for dispensing medication, for example, responsive to input from sensor 18.

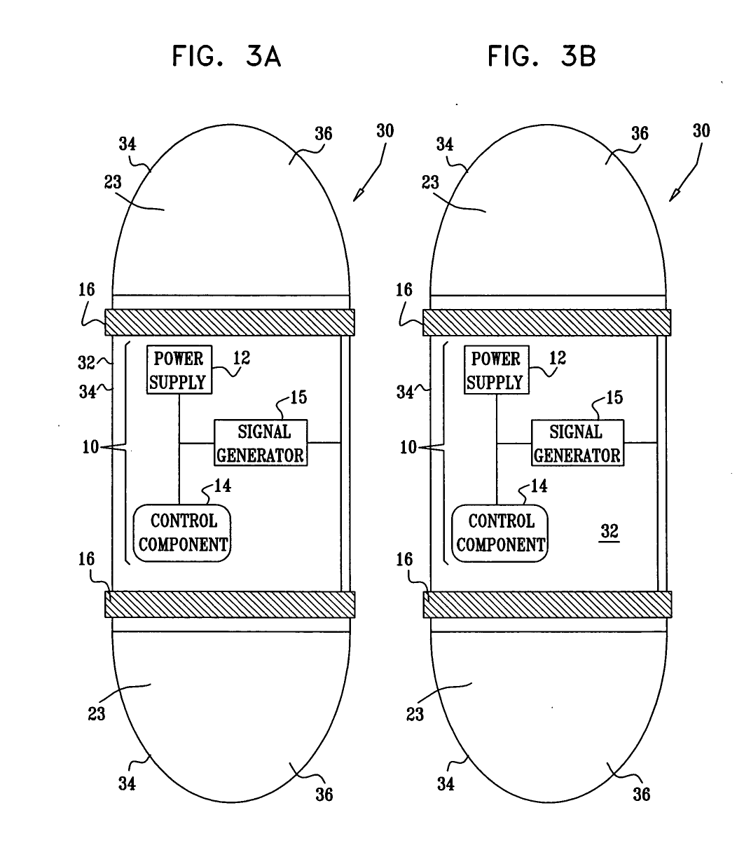

Reference is now made to FIGS. 3A and 3B, each of which illustrates an ingestible, electrically-assisted, drug-delivery system 30, in accordance with embodiments of the present invention. System 30 comprises device 10, enclosed within a biocompatible, biologically inert housing 32, formed for example, of stainless steel or silicone, or another biocompatible, inert material. Device 10 of the present embodiment typically comprises at least power supply 12, control component 14, signal generator 15, and at least two electrostimulating electrodes 16, for providing electrotransport.

In the embodiment shown in FIG. 3A, housing 32 of device 10 defines an internal cavity in which components of device 10 are located. In the embodiment shown in FIG. 3B, housing 32 defines no cavity; rather, it is formed as a cast, for example of silicone, wherein components of device 10 are imbedded.

System 30 further comprises a drug 36, attached to device 10 and enclosed by a sheath 34, which encapsulates both device 10 and drug 36. Alternatively, sheath 34 encapsulates only drug 36. Drug 36 is held in drug-dispensing cavities 23, which typically are formed at two ends of system 30, or at one end. Sheath 34 typically comprises a biologically compatible, biologically inert polymeric material, such as cellulose acetate or ethyl cellulose, that allows diffusion of drug 36 to the GI tract. Alternatively, sheath 34 is formed of a mixture of water-soluble particles in a water-insoluble matrix, such as polyvinyl acetate, or acrylic acid copolymers, so that the water soluble particles dissolve in the GI tract, leaving micropores in matrix, and drug 36 diffuses through the micropores. Alternatively, sheath 34 is formed of biologically-degradable material, which degrades when in contact with water, or at a specific pH value, so as to release drug 36 to the GI tract, where drug 36 travels with device 10 until the drug is absorbed. For example, the biologically-degradable material may comprise hydroxypropylcellulose or glycerol behenate. As system 30 travels in the GI tract, electrodes 16 of device 10 provide for electrotransport, which enhances absorption across the intestinal epithelium.

In accordance with some embodiments of the present invention, the electrotransport may include any one of, or a combination of, iontophoresis, electroosmosis, and electrophoresis, which enhance diffusion processes through the epithelial cells, and, for some applications, additionally electroporation, which, typically using high voltage, creates transient permeable structures or micropores in the epithelial cell membranes, enabling passage of large molecules through the epithelium.

In accordance with some embodiments of the present invention, the electrotransport is facilitated by applying a “low intensity time-varying” (LITV) signal, as defined hereinabove.

For some applications, appropriate electrostimulation parameters may include a DC voltage of up to 3 volts, or square pulses of up to 3 volts at a low frequency of 1-50 Hz. These parameters are typically appropriate for iontophoresis. Alternatively, the parameters may include an AC voltage of between about 3 and about 50 Volts, at a frequency of between about 1 and about 300 Hz. These parameters are typically appropriate for electroporation. Further alternatively, such as for applying a LITV signal, the electrostimulation may be applied as a series of pulses, with parameters including (a) a current of less than about 5 mA, (b) a frequency of between about 1 and about 10 Hz, or between about 10 and about 100 Hz, (c) a pulse duration of between about 0.1 and about 1 millisecond, or between about 1 and about 10 milliseconds, and (d) a stimulation period of between about 1 and about 15 minutes, or between about 15 and about 120 minutes. The pulses may be monophasic or biphasic. The LITV signal is typically sufficiently weak so as not to cause local activation of smooth muscle, which may interfere with normally-occurring peristaltic movement. Application of a current of less than about 5 mA typically results in a voltage of between about 0.1 and about 8 Volts/cm (e.g., between about 0.5 and about 5 Volts/cm), depending upon the surface area of the electrodes, the portion of the GI tract to which drug 36 is to be delivered, the content of the GI tract, the individual physiology of the patient (e.g., of the patient's GI wall tissue), and other factors.

For some applications, the LITV signal is applied in a low-frequency train of high-frequency bursts. Typically, the train has a repetition frequency of between about 6 and about 30 Hz, i.e., between about 6 and about 30 bursts are applied per second. Each burst typically includes between 1 and about 4 pulses, with a delay of about 4 to about 8 milliseconds between the start of each successive pulse (i.e., a frequency of pulses within a burst of between about 125 and 250 Hz). Each pulse typically has a duration of between about 0.1 and about 2 milliseconds.

For some applications, a DC or low-frequency square-pulse voltage and an AC voltage are superimposed, in order to facilitate a combination of two or more electrotransport processes.

It will be appreciated that signals of other shapes and (or) duty cycles may similarly be used. Furthermore, the aforementioned parameters are provided as examples; in accordance with embodiments of the present invention, other parameters, which may be higher or lower, may be used.

It will be appreciated that, in general, electrotransport parameters appropriate for the transport of drugs across the epithelial cells of the GI tract are lower than parameters appropriate for transdermal drug transport, as the GI tract lacks the stratum corneum barrier found in the skin.

In an embodiment of the present invention, the stimulation parameters are selected based at least in part on:
the particular properties of drug 36. Drugs comprising larger molecules typically require stronger stimulation. For example, when the electrotransport is facilitated by applying an LITV signal, stronger stimulation may be provided by stimulating with longer pulses, longer pulse trains of more pulses, and/or at higher voltages. In addition, even longer pulses may be used to increase the absorption of drugs having charged molecules.
the portion of the GI tract to which drug 36 is to be delivered. For example, intrinsic absorption characteristics of the jejunum are different from those of the ileum. As a result, stimulation with the same parameters generally results in greater absorption in the jejunum than in the ileum. Therefore, for some applications, stronger stimulation is applied when drug 36 is released in the ileum than in the jejunum.

For some applications, parameters are selected that apply the lowest amount of energy sufficient to achieve drug passage through the GI tract wall. The use of higher energy levels may in some cases increase the possibility of local irritation of the epithelial tissue (although actual damage to the tissue is unlikely even at the higher end of the range of energies used). In addition, lower energy levels may enable a longer stimulation period and increased drug absorption. Such increased drug absorption may allow a lower dosage of the drug, which may reduce the cost of the drug and/or the size of drug-delivery system 30 for some applications.

Alternatively, for other applications, parameters are selected that apply greater than this lowest amount of energy.

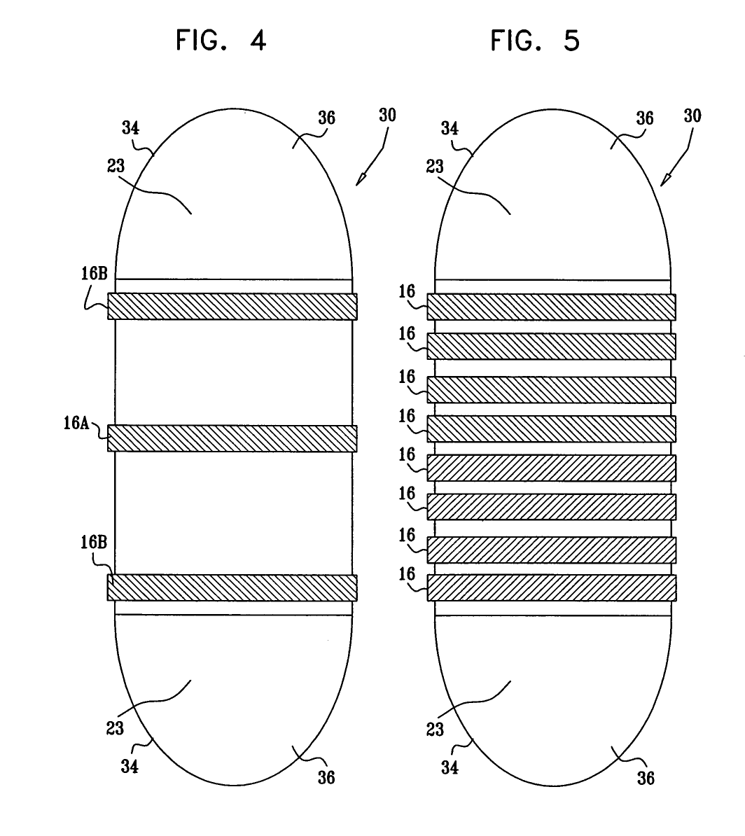

Reference is now made to FIGS. 4 and 5, which illustrate ingestible, electrically-assisted, drug-delivery systems 30, in accordance with embodiments of the present invention. In these embodiments, drug-delivery system 30 comprises a plurality of electrodes 16. For example, in the configuration shown in FIG. 4, system 30 comprises a single cathode 16A and two anodes 16B, or a single anode 16A and two cathodes 16B. Alternatively, as shown in FIG. 5, system 30 comprises a plurality of anodes and cathodes 16.

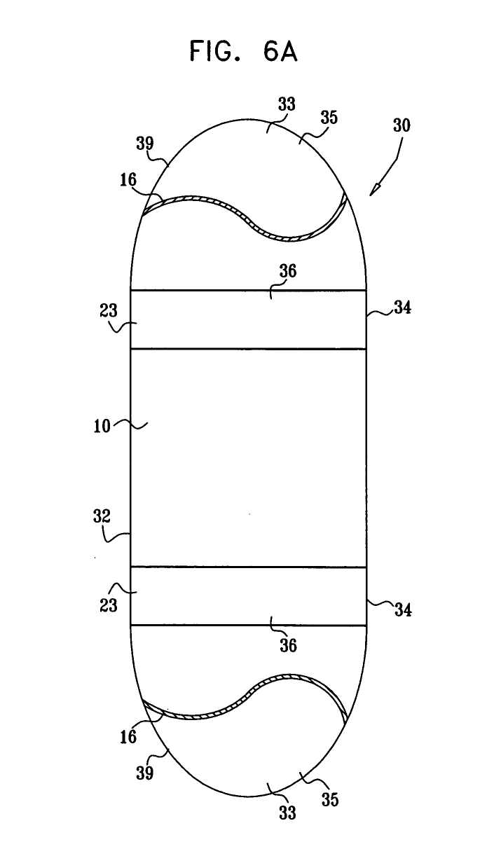

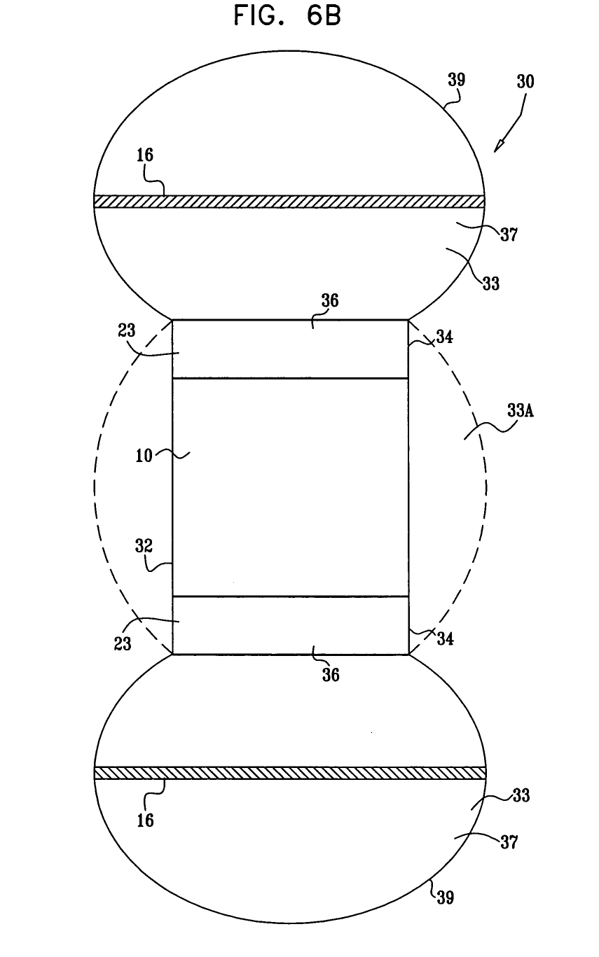

FIGS. 6A and 6B illustrate ingestible, electrically-assisted, drug-delivery system 30 in respective resting and drug-delivery phases thereof, in accordance with an embodiment of the present invention. In this embodiment, device 10 comprises self-expansible portions 33, enclosed in a biologically-inert and biocompatible elastic film 39, such as natural or synthetic thin rubber. For some applications, electrodes 16 are painted on elastic film 39, for better contact between electrodes 16 and the GI walls. The self-expansible effect may be produced, for example, by a chemical reaction of a substance 35 (FIG. 6A), that produces a gas 37, such as CO₂ (FIG. 6B). In the present embodiment, drug-dispensing cavities 23 may be located between self-expansible portions 33 and the main body of device 10. For some applications, system 30 of the present embodiment is used to facilitate contact between electrodes 16 and the GI walls of the colon.

For some applications, device 10 comprises a central portion 33a comprising a self-expansible portion, disposed between self-expansible portions 33 that have electrodes 16 thereon. Typically, portion 33a is adapted to expand until it contacts the inner wall of the gastrointestinal tract. Thus, portion 33a is typically able to expand to at least the same diameter as self-expansible portions 33, and thereby inhibit current flow in the fluid of the lumen of the gastrointestinal tract, and (for constant voltage) facilitate higher current flow in the tissue of the gastrointestinal tract itself. As appropriate, similar central self-expansible portions may be integrated into the embodiments of the invention described with reference to one or more of the other figures of the present patent application.

Alternatively, portion 33a does not comprise a self-expansible portion, but is instead in the state shown by the dashed lines in FIG. 6B prior to being ingested by the subject. In this case, portion 33a is pre-sized to be of a diameter suitable for contacting the inner wall of the gastrointestinal tract in a region of the gastrointestinal tract where drug delivery is desired. As appropriate, similar central portions 33a may be integrated into the embodiments of the invention described with reference to one or more of the other figures of the present patent application.

For some applications, an outer surface of portion 33a comprises a hydrophobic and/or lipophilic material, to minimize the extent to which current flowing between electrodes 16 passes within the gastrointestinal tract lumen itself. In an embodiment, portion 33a comprises the hydrophobic and/or lipophilic material, and has a smaller diameter than self-expansible portions 33.

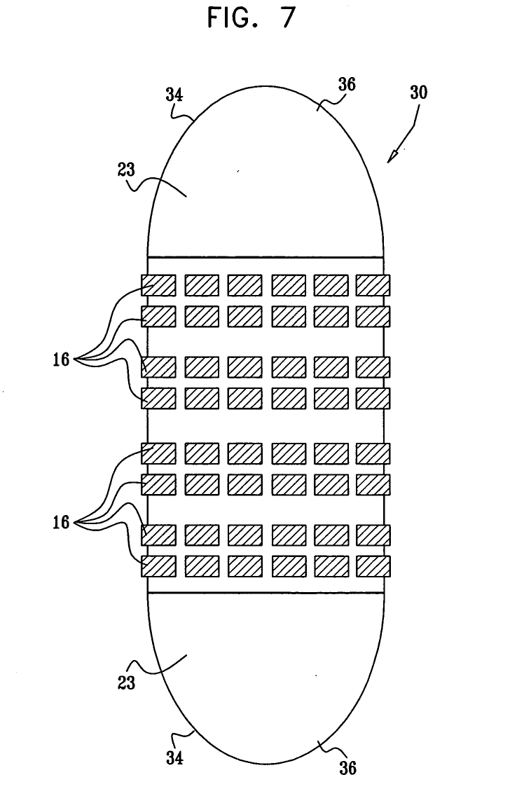

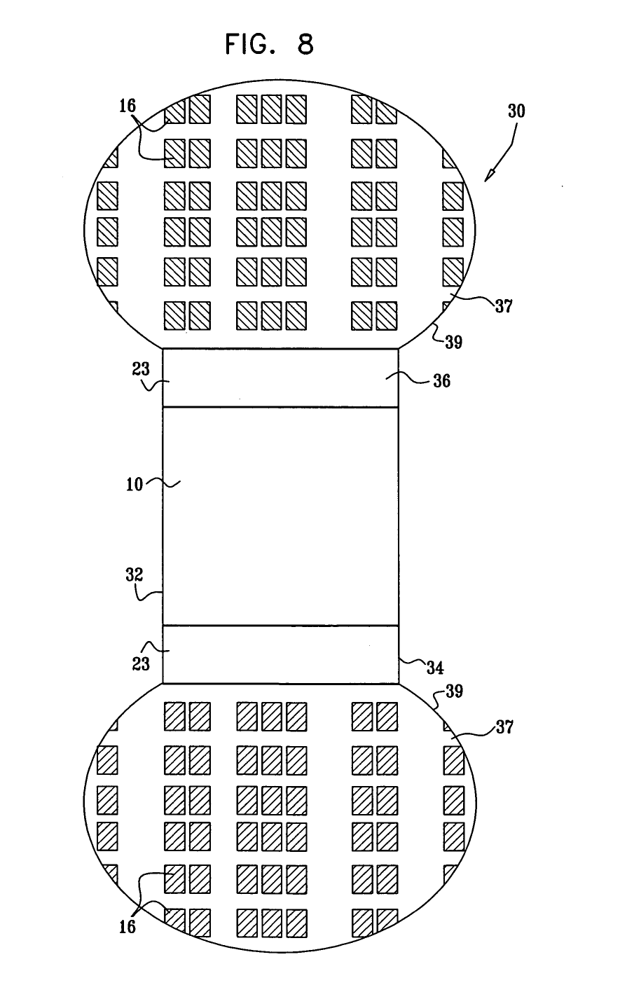

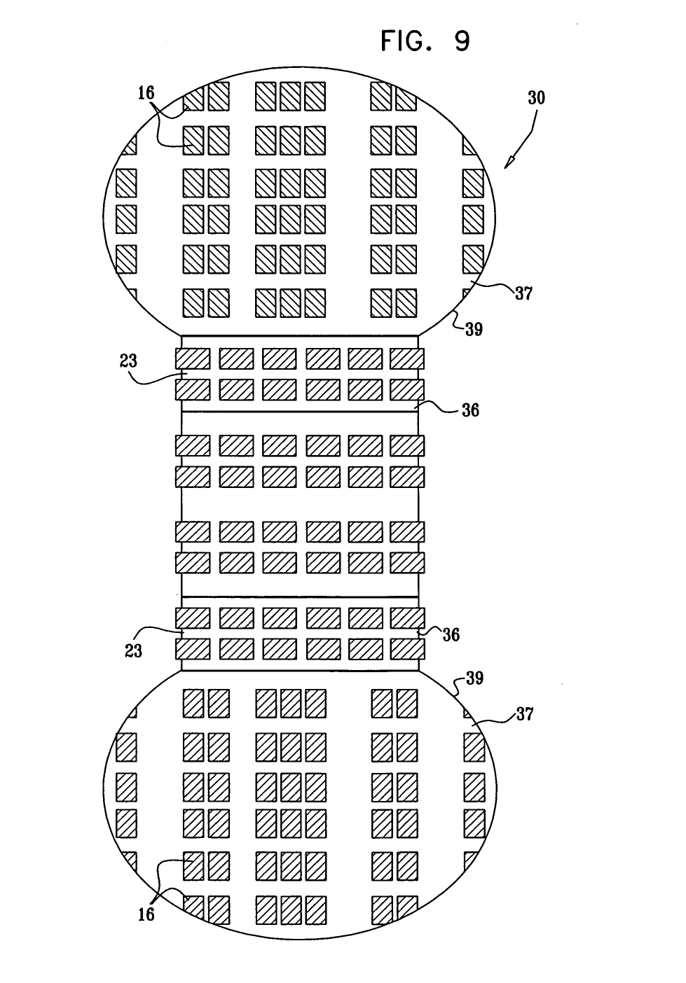

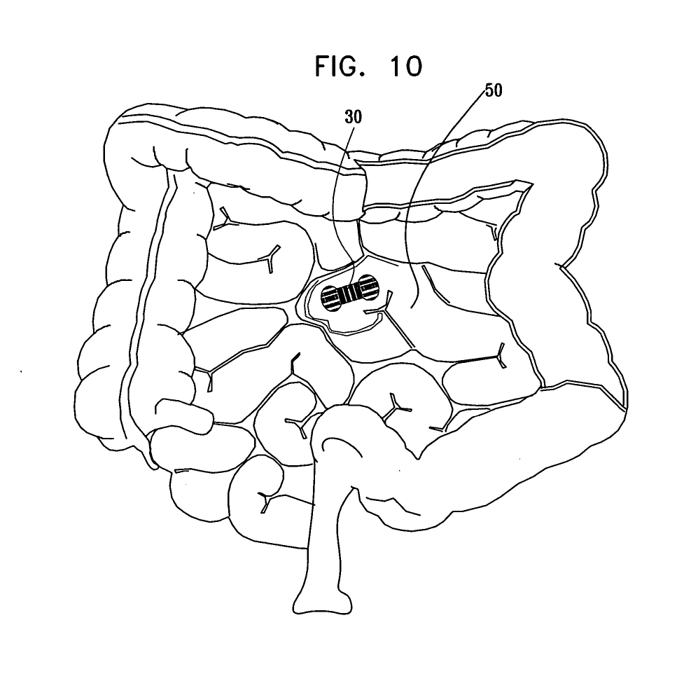

FIGS. 7, 8, and 9 illustrate ingestible, electrically-assisted, drug-delivery systems 30, in accordance with embodiments of the present invention. In these embodiments, system 30 comprises a plurality of electrodes 16 and self-expansible forms.

FIG. 10 illustrates ingestible, electrically-assisted, drug-delivery system 30, as it travels in a GI tract 50, in accordance with an embodiment of the present invention. Both the self-expansible portions of system 30 and the plurality of electrodes 16 that cover its exterior are operative to facilitate sliding contact between walls of GI tract 50 and system 30, as suitable for electrostimulation.

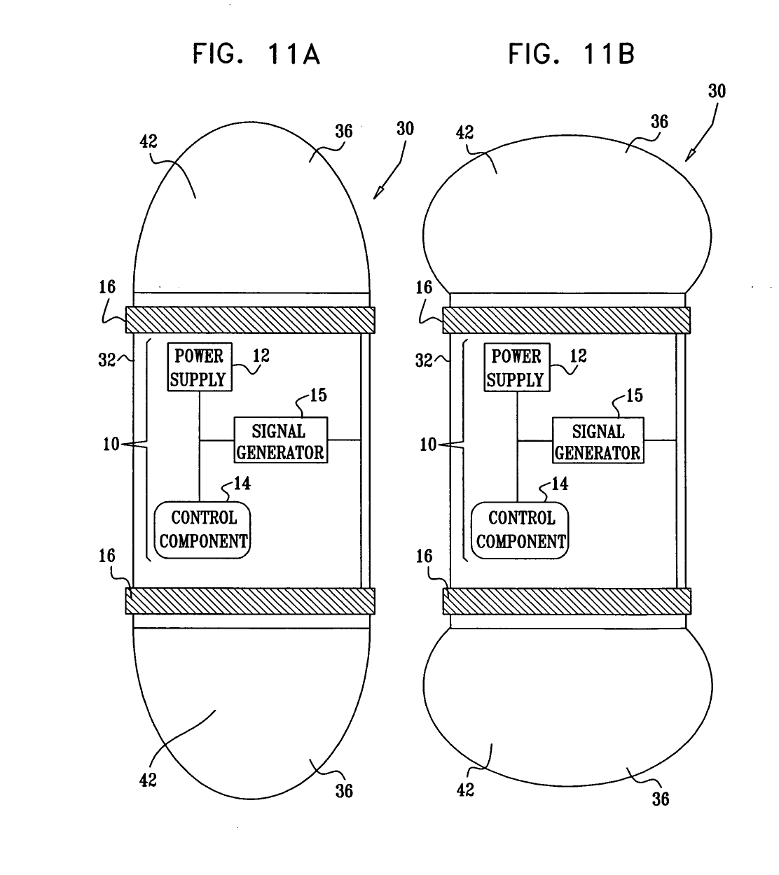

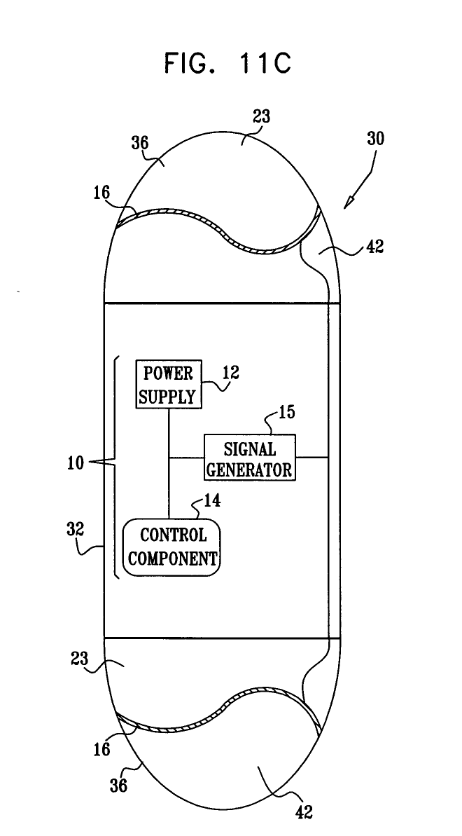

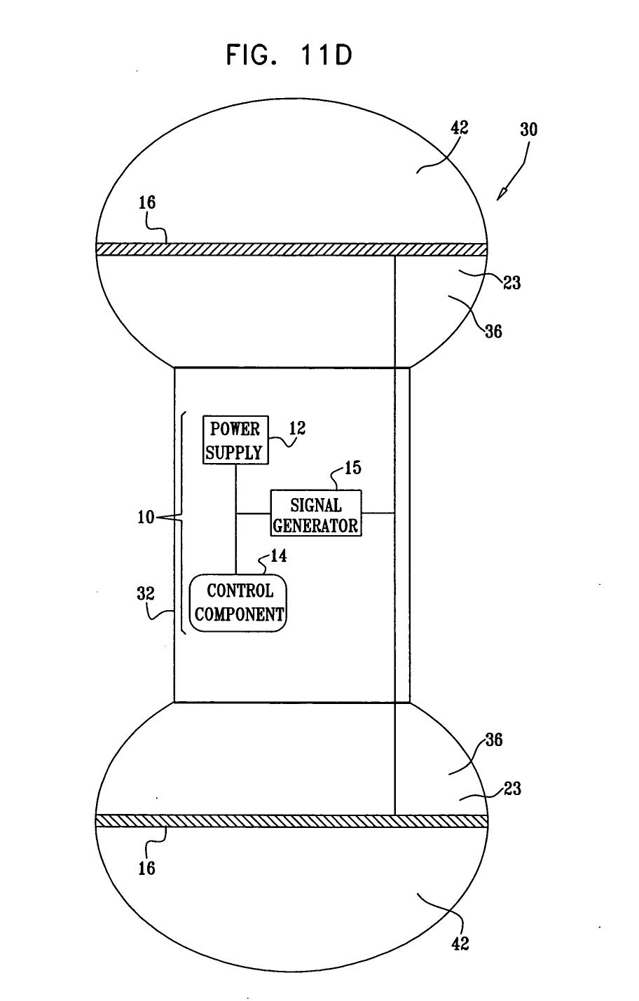

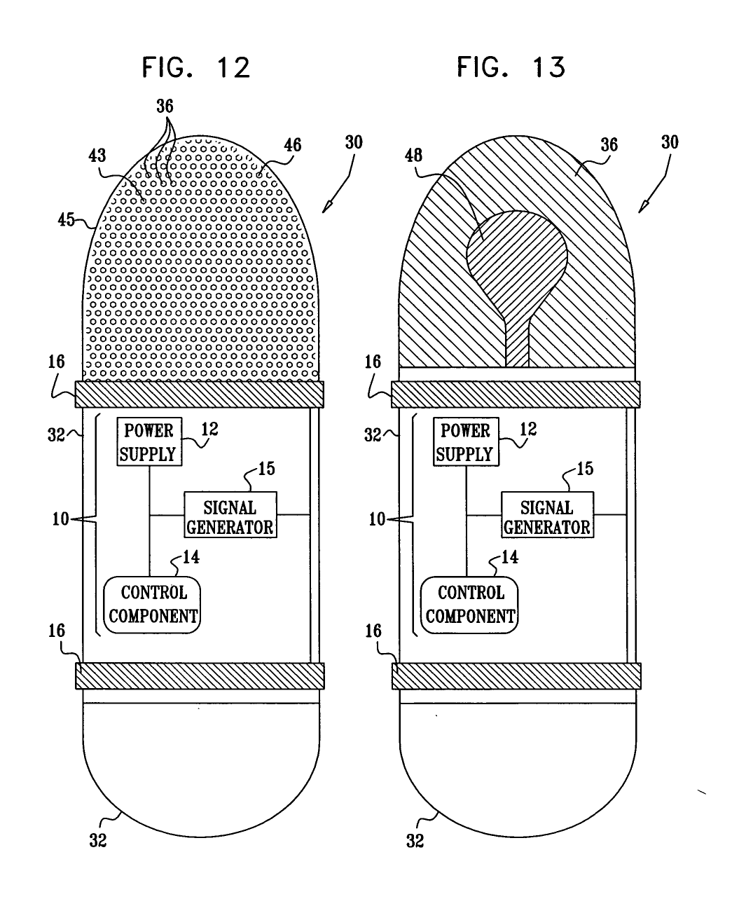

FIGS. 11A-11D illustrate ingestible, electrically-assisted, drug-delivery system 30, in accordance with embodiments of the present invention. In these embodiments, a self-expansible drug matrix is used. Typically, drug 36 is enclosed by a swelling polymer 42, which may be biodegradable, such as hydroxypropylmethylcellulose-HPMC or POLYOX™ (manufactured by The Dow Chemical Company), which expands when brought into contact with GI fluids. Typically, the drug is mixed with the swelling polymer, so as to swell with it.

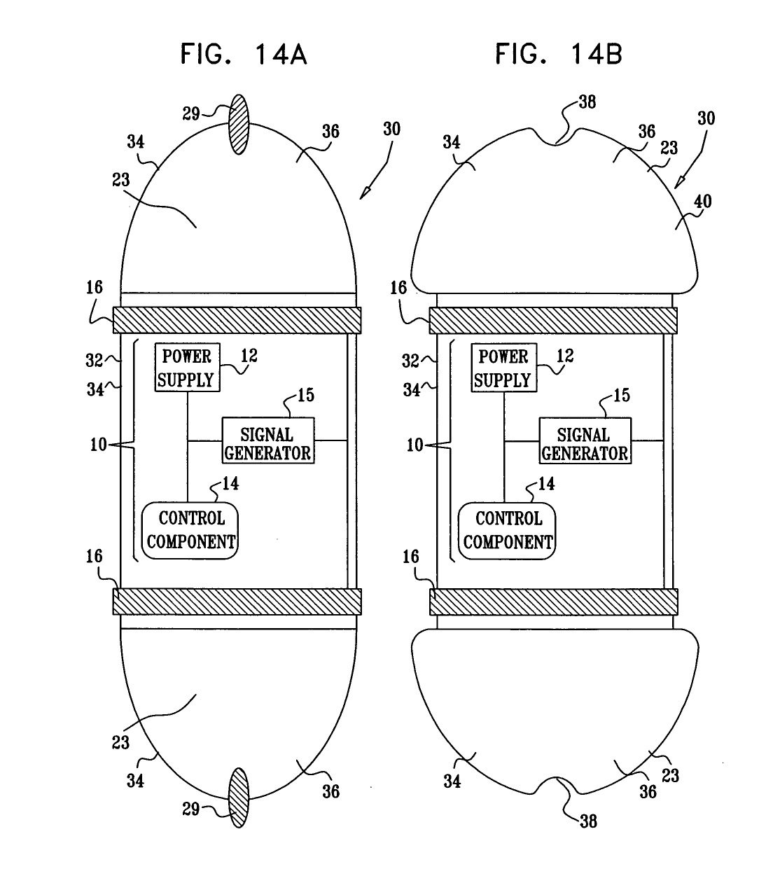

FIG. 12 illustrates ingestible, electrically-assisted, drug-delivery system 30, formed as a capsule 45, and containing drug 36, as micropellets 43, in accordance with an embodiment of the present invention. A biodegradable film 46 encapsulates micropellets 43. As film 46 disintegrates in the GI tract, drug 36, in the form of micropellets 43, is released.

FIG. 13 illustrates ingestible, electrically-assisted, drug-delivery system 30, in accordance with an embodiment of the present invention. In this embodiment, no film is used to contain drug 36. Rather, drug 36 is pressed onto a biocompatible solid bar 48, and slowly dissolves in the GI tract.

FIGS. 14A and 14B illustrate ingestible, electrically-assisted, drug-delivery system 30 in respective resting and drug-delivery phases thereof, in accordance with an embodiment of the present invention. In this embodiment, drug delivery occurs by osmosis. As a water-soluble plug 29 (FIG. 14A) dissolves, an orifice 38 is opened (FIG. 14B). Uptake of water into drug-dispensing cavity 23 increases the osmotic pressure within the system. The build-up of the osmotic pressure gradient drives the drug through orifice 38 in a controlled manner.

Alternatively, sheath 34 of drug 36 may be formed as cellulose acetate combined with polyethylene glycol (PEG). After ingestion the PEG dissolves, leaving the drug 36 coated with a semi-permeable membrane that controls the release of the drug by osmotic mechanism. Osmognate additives, such as NaCl, added to the drug core, and/or perforation of the sheath 34, may contribute to better controlling the release patterns (osmognates are materials, usually salts, with high solubility and the ability to create high osmotic pressure, to attract water).

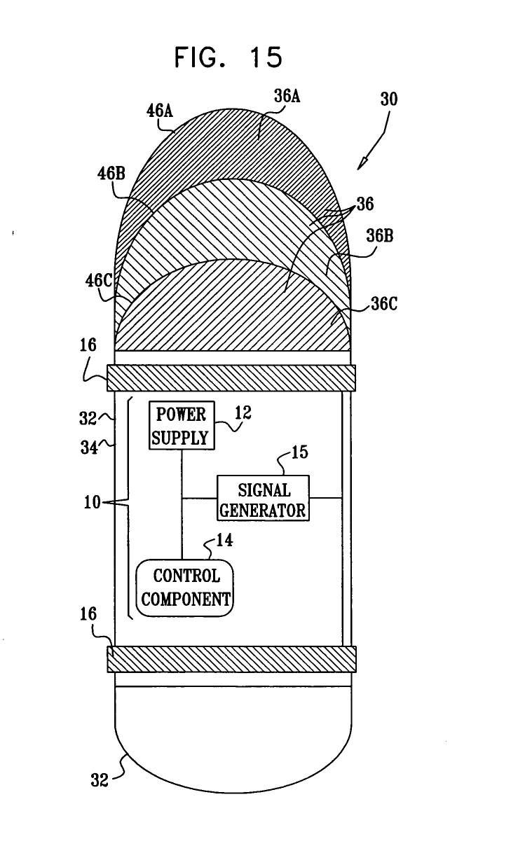

FIG. 15 illustrates ingestible, electrically-assisted, drug-delivery system 30, in accordance with an embodiment of the present invention. In this embodiment, drug release is pH-dependent. Drug 36 is enclosed by at least one film 46A, which dissolves at a specific pH value. For some applications, the pH value is selected to be in the range commonly found in the small intestine, e.g., between about 4.7 and about 6.5, in order to release drug 36 into the small intestine, while substantially preventing the earlier release of the drug in the stomach. Alternatively, the pH is selected to be in the range commonly found in another portion of the GI tract, such as the large intestine. (See Table 1 of the Background Section for exemplary pH values.) For other applications, the pH value is selected to be in the range commonly found in the stomach, e.g., between about 1.2 and about 3.5, such that film 46A dissolves in the stomach, releasing at least a portion 36A of drug 36. Optionally, system 30 comprises a second film 46B, which dissolves at a pH characteristic of a more distal portion of the GI tract, such as the small intestine, releasing a second portion 36B of drug 36 therein. Further optionally, system 30 comprises a third film 46C, which dissolves at a pH characteristic of a still more distal portion of the GI tract, such as the large intestine (e.g., a pH value of between about 7.5 and about 8.0 for the large intestine), thereby releasing a third portion 36C of drug 36. In this manner, specific drug portions, or even different drugs 36A, 36B, and 36C may be targeted to different portions of the GI tract. Alternatively or additionally, the pH values are selected to release a first portion of drug 36 in the small intestine, and a second portion in the large intestine.

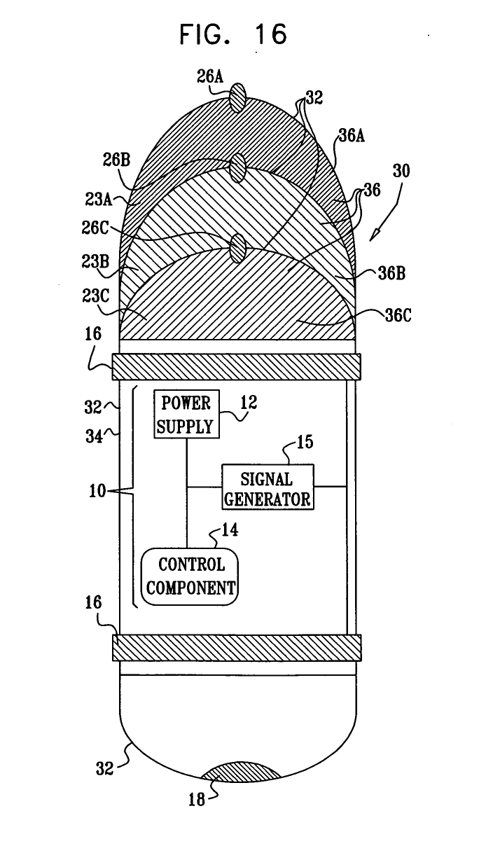

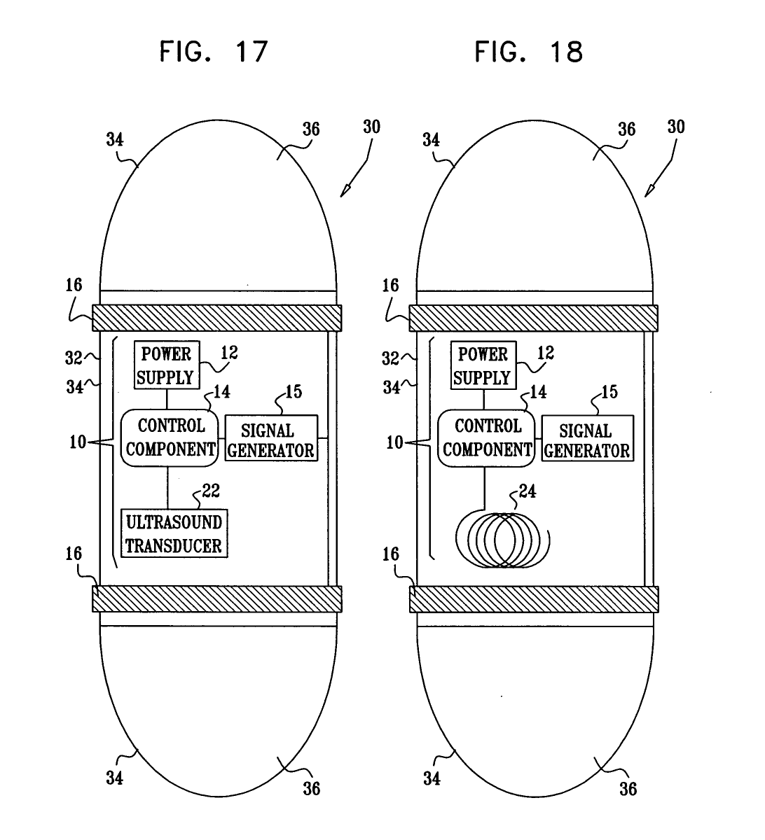

FIG. 16 illustrates ingestible, electrically-assisted, drug-delivery system 30, in accordance with an embodiment of the present invention. In this embodiment, drug release is pH-dependent. Drug 36 is enclosed by housing 32, in two or more drug-dispensing cavities, such as three drug-dispensing cavities 23A, 23B, and 23C, sealed respectively by three electronic valves 26A, 26B, and 26C, the operation of which is controlled by control component 14. A pH sensor 18 typically senses a specific pH value or range of values, and transmits the information to control component 14, which opens one or more of valves 26A, 26B, and 26C, responsive to the sensing.

FIG. 17 illustrates ingestible, electrically-assisted, drug-delivery system 30, in accordance with an embodiment of the present invention. In this embodiment, device 10 comprises ultrasound transducer 22 for providing sonophoresis as a drug transport mechanism. It will be appreciated that sonophoresis may be applied alone, or in combination with electrotransport, using electrodes 16.

FIG. 18 illustrates ingestible, electrically-assisted, drug-delivery system 30, in accordance with an embodiment of the present invention. In this embodiment, device 10 comprises ablation apparatus 24 for providing ablation, such as RF ablation, as a drug transport mechanism. It will be appreciated that ablation may be applied alone, or in combination with electrotransport, using electrodes 16.

Typically, RF ablation parameters include frequencies of about 50 to about 150 kHz, and potentials of about 3-100 volts. These parameters are provided as examples; in accordance with embodiments of the present invention, other parameters, which may be higher or lower, may be used.

Alternatively, ablation apparatus 24 performs microwave ablation, laser ablation, cryogenic ablation, thermal ablation, or liquid jet ablation.

FIG. 19 illustrates ingestible, electrically-assisted, drug-delivery system 30, in accordance with an embodiment of the present invention. In this embodiment, device 10 comprises telemetry system 20, for providing communication with an extracorporeal station 21 (FIG. 2). For example, sensor 18 may transmit to extracorporeal station 21 temperature values along the GI tract. These values may be used to inform a person using system 30 of a sudden, or localized temperature increase, suggestive of a problem. Alternatively, sensor 18 may comprise a pH sensor, and extracorporeal station 21 may be used to remotely control valves, such as valves 26A, 26B, and 26C of FIG. 16.

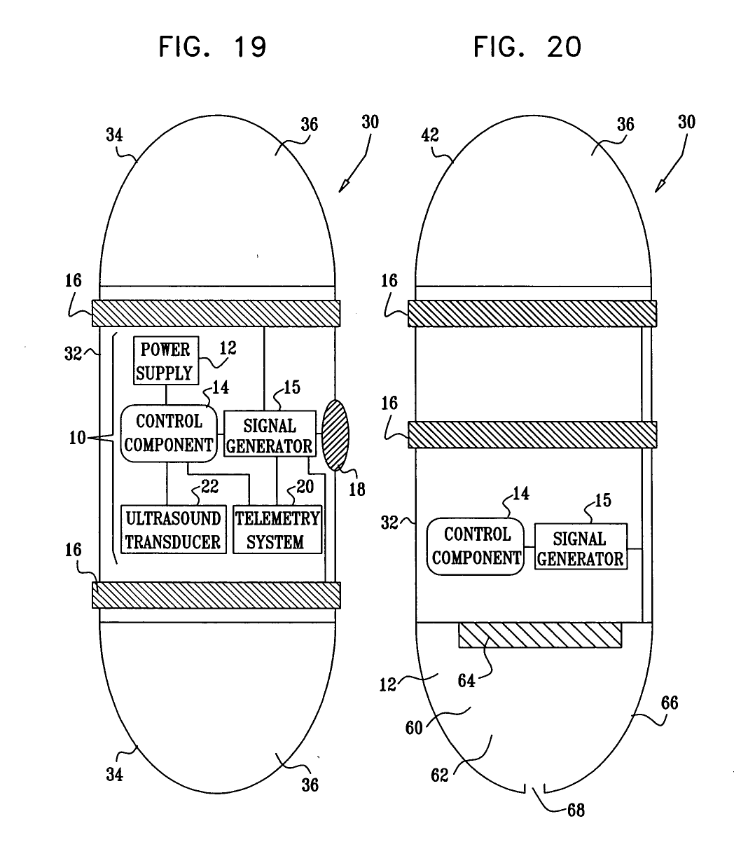

FIG. 20 illustrates ingestible, electrically-assisted, drug-delivery system 30, in accordance with an embodiment of the present invention. In this embodiment, power supply 12 of device 10 is constructed as a galvanic cell 60, comprising an anode 64, a cathode 66, and an orifice 68. As system 30 travels through the GI tract, GI fluids 62 enter galvanic cell 60 via orifice 68, and serve as the electrolyte for the cell.

When the half-life of a drug is less than desired, a controlled release dosage form may be designed, to reduce fluctuation in plasma drug concentration and to provide a more uniform therapeutic effect. Oral controlled-release forms are often designed to maintain therapeutic drug concentrations for at least 12 hours. Several controlled release mechanisms may be used, for example, as taught by Encyclopedia of Controlled Drug Delivery, volume 2, edited by Edith Mathiowitz, pp. 838-841. These are based on the use of specific substances, generally polymers, as a matrix or as a coating. These may be materials that degrade fast or slowly, depending on the desired effect.

In accordance with embodiments of the present invention, drug 36 is released in a controlled manner, using one or more of the following techniques:
The drug, which may be solid, liquid or a suspension in liquid, may be encapsulated in a polymeric material, so that drug release is controlled by diffusion through the capsule walls.
The drug particles may be coated with wax or poorly soluble material, or an insoluble material (e.g., polyvinyl chloride) mixed with a water-soluble, pore forming compound, so that drug release is controlled by the breakdown of the coating.
The drug may be embedded in a slow-release matrix, which may be biodegradable or non-biodegradable, so that the drug release is controlled by diffusion through the matrix, erosion of the matrix, or both.
The drug may be complexed with ion-exchange resins that slow down its release.
The drug may be laminated, as a jellyroll, with a film, such as a polymeric material, which may be biodegradable or nonbiodegradable, so that the drug is released by diffusion, erosion or both.
The drug may be dispersed in a hydrogel, or a substance that forms a hydrogel in the GI tract, so that the drug release is controlled by diffusion of the drug from the water-swollen hydrogel.
Osmotic pressure may be used to release the drug in a controlled manner. Uptake of water into the dosage unit increases the osmotic pressure within the system. The build-up of the osmotic pressure gradient drives the drug through one or more orifices in the dosage form to release the drug in a controlled manner.
The drug may be formed as micropellets, of a density that is lower than that of the GI fluid. The micropellets may float for a long time, before dissolution.
The drug may contain a bioadhesive polymer that adheres to the surface of the epithelium, to extend the time of the drug in the GI tract.
The drug may be chemically bonded to a polymer and released by hydrolysis.
Macromolecular structures of the drug may be formed via ionic or covalent linkages, which control the drug release by hydrolysis, thermodynamic dissociation or microbial degradation.
The drug may be coated with a combination of a soluble and insoluble polymers. When the soluble particles dissolve, they form a microporous layer around the drug core, so that the drug may permeate slowly through the micropores. The rate of release depends on the porosity and thickness of the coating layer. The coating layer components can be varied to prolong release of the drug until the dosage unit is in the presence of a specific pH (e.g., for colon targeting).
The drug may be laminated with a layer designed to dissolve at a specific pH value, for targeting a specific portion of the GI tract.
The drug may be laminated with several layers, each designed to dissolve at a different specific pH value, for targeting different portions of the GI tract, for example, for targeting the colon.
The drug may be designed for pH-independent controlled release, and produced by wet granulating an acidic or basic drug blend with a buffering agent and the appropriate excipients, wherein the granules are then coated with a film, which is permeable in GI fluid and compressed into tablets. Upon oral administration, GI fluid permeates the film coating, and the buffering agents adjust the pH value of the tablet so that the drug can dissolve and permeate out of the dosage form at a constant rate, independent of the pH level in the GI tract.
The drug formulation may be sealed in the insoluble capsule body by means of a water-soluble plug and a hydrogel plug. When the capsule is swallowed, the water-soluble plug dissolves in the gastric juice and exposes the hydrogel plug, which begins to swell. At a predetermined time after ingestion, the hydrogel plug is ejected and the encapsulated drug formation is then released into the alimentary tract.

Alternatively or additionally, other controlled release means known in the art are used.

As appropriate, some or all portions of the capsule are configured to be biodegraded by bacteria in the patient's colon.

It will be appreciated that in accordance with embodiments of the present invention drug release may take any of the following options: controlled release, delayed release, pulsatile release, chronotherapeutic release, immediate release, enterocoated release (activation starts at the small intestine, and the pH-dependent coating protects from the gastric acidic environment). The dosage forms may be chronotherapeutic (adaptation to the circadian rhythm) or colonic delivery type, based on multiple coatings system. The drug may be formed as a capsule of hard gelatin, as compressed powder, or as any other alternative known in the art, for example, hydroxypropyl methylcellulose (HPMC).

When the drug is a peptide formulation or a protein drug, functional additives may be used in order to enable oral delivery. Typical entities are: protease inhibitors, stabilizers, absorption enhancers, and PGP inhibitors, such as verapamil or quinidine.

Additionally, various additives may be used with drug 36. These may include protease inhibitors, which shield against luminal brush, border peptidases, such as Trypsin inhibitor, Chemostatin, Bowman Birk Inhibitor, Aprotinin, SBTI, and polycarbophyl.

Additionally, absorption enhancers, such as NSAIDs, decanoic acid, sodium salicylate, SLS, quaternary ammonium salts, Bile salts-na-cholate, octanoic acid, glycerides, saponins, and/or medium chain fatty acids may be used.

It will be appreciated that in many cases chemical enhancers interact with peptides and proteins. An advantage of some embodiments of the present invention is the ability to circumvent this interaction, by using electrically assisted absorption, in place of chemical enhancers.

Additionally, stabilizers, such as proteins, sugars, polyols, amino acids, inorganic salts, and/or surfactants, may be used.

Furthermore, other pharmaceutically adjuvant for peptides such as buffering agents and/or antioxidants may be used.

Suitable polymers for matrix formation for controlled or slowed release of oral drugs include Acrylates, acrylic acid copolymers, Eudragit, RL/RS type, cellulose derivatives like ethyl cellulose, HPMC, carboxymethylcellulose, carbomers, cellulose acetate, PVA, gums, and any other pharmaceutically acceptable polymers.

In addition to polymers, certain types of lipids may serve as matrix formers as well, for example, glycerol behenate, or glycerol monostearate.

It will be appreciated that the matrix forming polymers may be filled into capsules or compressed into tablets.

Suitable polymers for functional coatings of oral drugs for controlled or slowed drug release include Ethocel (ethyl cellulose), HPMC, Kollicoat (PVA, PVP combinations), CA esters, Eudragits, and enteric coating (pH-dependent) type polymers (Eudragit L,S, CAP, HPMCP, etc.). In addition, acceptable pharmaceutical fillers like MCC, lactose, and ca-phosphate may be used as well.

These coatings may be applied to both tablets and capsules.

It will be appreciated that the type of coating will be determined according to the drug and the desired release profile, such as slow release, enteric (mainly for peptide type), chronotherapeutic, colonic, osmotic, etc.

It will be further appreciated that the coating may be additional to matrix-based dosage forms, either for tablets or for capsules.

Drug candidates for some embodiments of the present invention include peptides, proteins, macromolecules, hormones, polar compounds, and poorly soluble compounds.

Some examples of drugs that may be used as drug 36, in accordance with embodiments of the present invention, include Interleukin 2, TGF-Beta 3, heparin, erythropoietin, cyclosporin, anticancer drugs, viral and non viral vectors for gene delivery, TNF, somatropin, interferones, copaxone, recombinant proteins, immune system modulators, monoclonal antibodies (Herceptin), vaccines, filgastrin, somatostatin, insulins, LHRH antagonists and analogs (Decapeptide, Leuprolide, Goseralin, calcitonin, triptorelin, oxytocin, and sandostatin.

Additionally, small molecule drugs, such as statins, immunosuppressants (e.g., sirolimus, tacrolimus), galantamine, celebrex, and other poorly soluble drugs, or drugs of low availability, may be used. These drugs may be Cox 2 inhibitors, CNS drugs, antibiotics, and any others that require improvement in their oral bioavailability.

Additionally, other known drugs of poor absorption may be used.

Reference is now made to the following examples, which together with the above descriptions illustrate embodiments of the invention in a non-limiting fashion.

### EXAMPLE 1

An electrically assisted, drug-delivery device 10.

Active drug: Insulin.

Filler: microcrystalline cellulose, lactose.

Protease inhibitor: chemostatin, trypsin inhibitor.

The components are mixed and compressed into tablets. An enterocoat is applied to protect from gastric environment. Eudragit L may be used.

### EXAMPLE 2

Similar to Example 1, but additionally including an absorption enhancer, such as decanoic acid.

### EXAMPLE 3

Capsule for oral delivery of copaxone, prepared as in Example 1. The components are dry-mixed and filled into capsules, which are coated with an enterocoat polymer like HPMCP.

### EXAMPLE 4

A tablet for controlled release of cyclosporin.

Both device 10 and HPMC and the drug substance are mixed together, and compressed into tablets (See FIG. 13). The complete system 30 is then coated with ethyl cellulose, which together with the HPMC delays and controls the drug release.

### EXAMPLE 5

An osmotic device. The tablet of Example 4 may be coated with cellulose acetate combined with PEG. After ingestion the PEG dissolves, leaving the tablet coated with a semi-permeable membrane that controls the release of the drug by an osmotic mechanism. Osmognate additives (defined hereinabove), such as NaCl, are added to the drug core, and perforation of the coating may contribute to better controlling the release patterns.

It will be appreciated that any known combination of drug-polymer, dosage form is acceptable, in accordance with embodiments of the present invention.

In accordance with some embodiments of the present invention, the electrically-assisted, drug-delivery system further comprises a visual imaging apparatus, for example, as described in U.S. Pat. No. 5,984,860 to Shan, U.S. Pat. Nos. 5,604,531 and 6,428,469 and U.S. patent application 2001/0035902, all to Iddan et al, all of which are incorporated herein by reference

In accordance with some embodiments of the present invention, the electrically-assisted, drug-delivery system further increases the dissolution rate of drugs that dissolve slowly. For example, sonophoresis which produces cavitation has an abrasive effect, and may be operative to enhance the dissolution of drugs of poor solubility.

In accordance with some embodiments of the present invention, the electrically-assisted, drug-delivery system is ingestible. Typically, it is free to pass through the GI tract. Alternatively, it may be tethered to a portion of the patient's body, e.g., to a tooth or to a band placed around the patient's head. Alternatively, the electrically-assisted, drug-delivery system may be mounted on a catheter.

In an embodiment of the present invention, the electrically-assisted, drug-delivery system comprises an endoscope (e.g., a colonoscope). The endoscope comprises the stimulation electrodes, while the other elements of the system (e.g., the power source and the control unit) are coupled to the endoscope and are typically adapted to remain outside the body. In this embodiment, the drug typically is administered in a liquid solution. The endoscope further comprises a drug delivery mechanism, such as a flexible tube attached to the endoscope. The distal end of such a tube is typically positioned to release the drug near the stimulation electrodes. For some applications, the system of this embodiment is used to deliver drugs to a specific site that is identified using conventional endoscopic functionality, e.g., that is identified visually using the endoscope. The stimulation electrodes and distal end of the drug-delivery tube are typically positioned near the distal end of the endoscope, in order to enable visual observation and targeting of drug release.

Embodiments of the present invention are designed to achieve previously unmet efficiency and bioavailability of orally delivered protein and peptide drugs. It will be appreciated that the electrically-assisted improvement may be performed in addition to and synergistically with known drug enhancers and stabilizers. In an embodiment of the present invention, synergistic drug absorption enhancement achieved using at least one of the electrical enhancement techniques described herein, in combination with a low concentration of a chemical enhancer, is greater than the sum of (a) the enhancement achievable with electrical enhancement technique alone and (b) the enhancement achievable with the low concentration of the chemical enhancer alone.

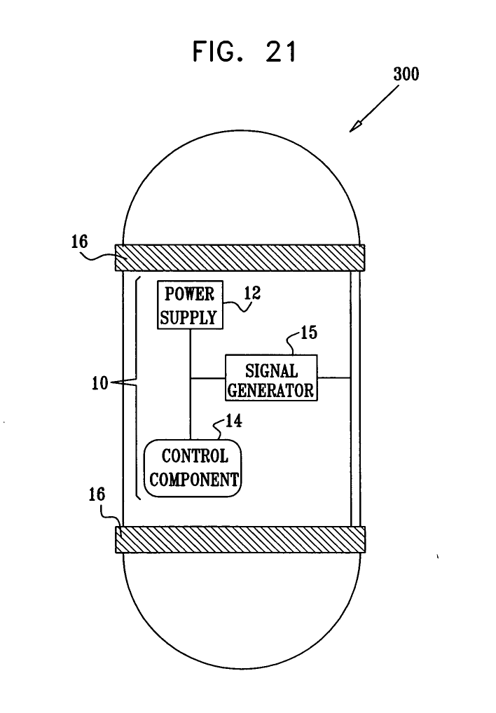

Reference is now made to FIG. 21, which is a schematic illustration of an ingestible, electrically-assisted drug-delivery facilitation system 300, in accordance with an embodiment of the present invention. System 300 is generally similar to drug-delivery system 30, described hereinabove with reference to FIGS. 3A and 3B, for example. System 300 comprises device 10, housing 32, power supply 12, control component 14, signal generator 15, and at least two electrostimulating electrodes 16. System 300 may employ any of the electrode configurations described hereinabove with respect to system 30, mutatis mutandis, such as those described with reference to FIGS. 4, 5, 6A, 6B, 7, 8, and 9.

However, unlike system 30, system 300 does not comprise drug 36. Instead, the patient typically ingests system 300 in conjunction with ingesting a commercially-available drug pill containing drug 36, e.g., before, simultaneously with, or after ingesting the drug pill. System 300 thus serves to enhance absorption of the drug released from the drug pill in the GI tract. For some applications, system 300 is configured to generally coordinate (e.g., synchronize) the application of electrostimulation with the expected release of the drug from the drug pill, such as by using one or more of the release-timing techniques described hereinabove. For example, system 300 may be coated with a controlled-release coating that generally matches the controlled-release timing of the drug pill. Numerous techniques for coordinating the electrostimulation with the drug release will be evident to those skilled in the art, having read the present patent application, and are within the scope of the present invention.

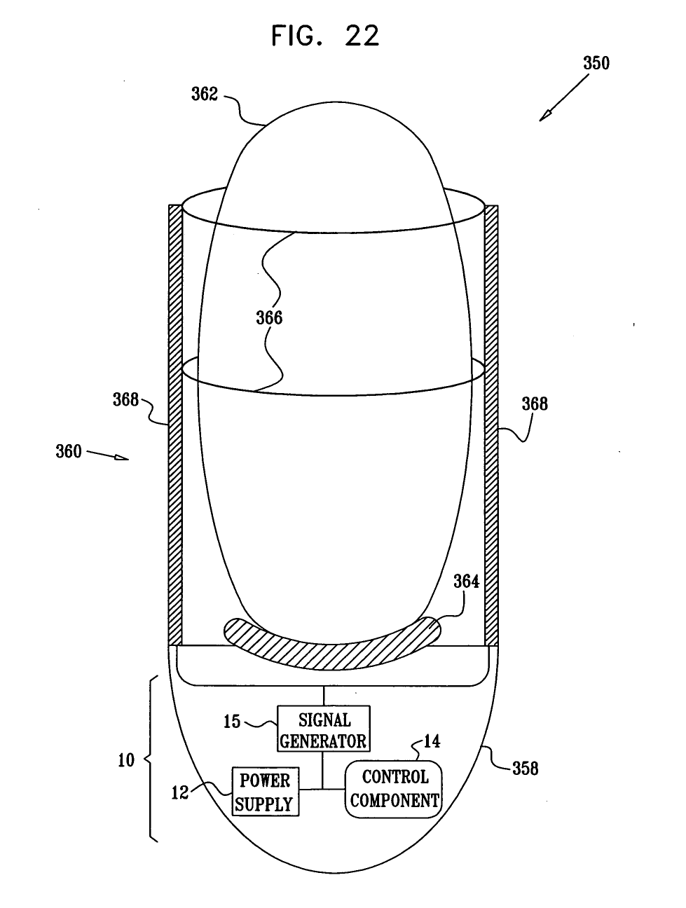

Reference is now made to FIG. 22, which is a schematic illustration of an ingestible, electrically-assisted drug-delivery system 350, in accordance with an embodiment of the present invention. System 350 is generally similar to drug-delivery system 30, described hereinabove with reference to FIGS. 3A and 3B, for example. System 350 comprises device 10, power supply 12, control component 14, and signal generator 15. These components are typically contained within a housing 358 of system 350. System 350 typically comprises an ingestible environmentally-sensitive mechanism, adapted to change a state thereof responsive to a disposition thereof within the GI tract.

However, unlike system 30, system 350 does not comprise drug 36. Instead, system 350 comprises a coupling mechanism 360, which is adapted to couple a commercially-available drug pill 362 to system 350. For some applications, coupling mechanism 360 comprises an adhesive 364, which holds pill 362 in place. Other coupling mechanisms, such as clips or other pressure-fitting mechanisms (configuration not shown), will be evident to those skilled in the art, having read the present patent application, and are within the scope of the present invention. Pill 362 may be coupled to system 350 by a manufacturer, the patient, or a healthcare worker, depending, for example, on medical, safety, commercial, or other considerations.

System 350 further comprises a drug-passage facilitation mechanism, which is adapted to facilitate passage of the drug contained in the drug pill through the epithelial layer of the GI tract. For some applications, the drug-passage facilitation mechanism comprises at least two electrostimulating electrodes 366. In the configuration shown in FIG. 22, electrodes 366 are configured such that they surround a portion of pill 362 once the pill has been coupled to system 350. The electrodes are typically supported by one or more electrically-insulated support elements 368. Alternatively, electrodes 366 are positioned elsewhere in the vicinity of pill 362, such as on housing 358. For example, system 350 may employ any of the electrode configurations described hereinabove with respect to system 30, mutatis mutandis, such as those described with reference to FIGS. 3A, 3B, 4, 5, 6A, 6B, 7, 8, and 9.

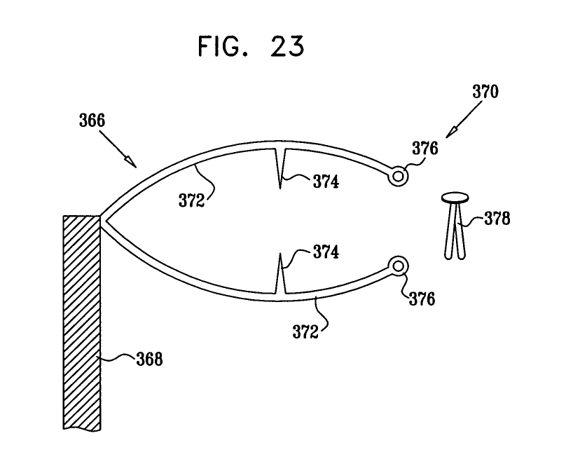

Reference is now made to FIG. 23, which is a schematic illustration of a coupling mechanism 370, in accordance with an embodiment of the present invention. In this embodiment, system 350 comprises coupling mechanism 370 alternatively or additionally to coupling mechanism 360 (FIG. 22). Coupling mechanism 370 comprises at least one of electrostimulating electrodes 366 (FIG. 22). The electrode comprises two substantially semicircular segments 372, each of which comprises or is shaped so as to define one or more spikes 374. Pill 362 (not shown in FIG. 23) is inserted between the segments, and distal ends 376 of the segments are brought together, thereby pressing spikes 374 into pill 362 and holding the pill in place. After insertion of the pill, distal ends 376 are typically held together, such as by a pin 378 that is inserted into the ends, or by another closing mechanism.

It is to be appreciated that the particular geometries shown in FIG. 23 are intended to provide another non-limiting example of ways in which a pill can be coupled to system 350. As appropriate, various components shown in FIG. 23 may be varied in size, position, or number, so as to facilitate the mounting of a pill to system 350.

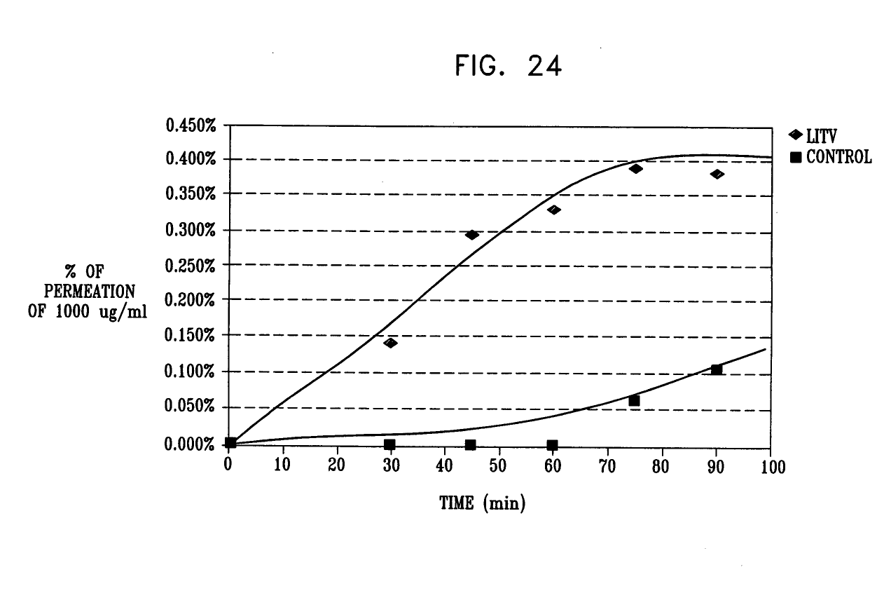

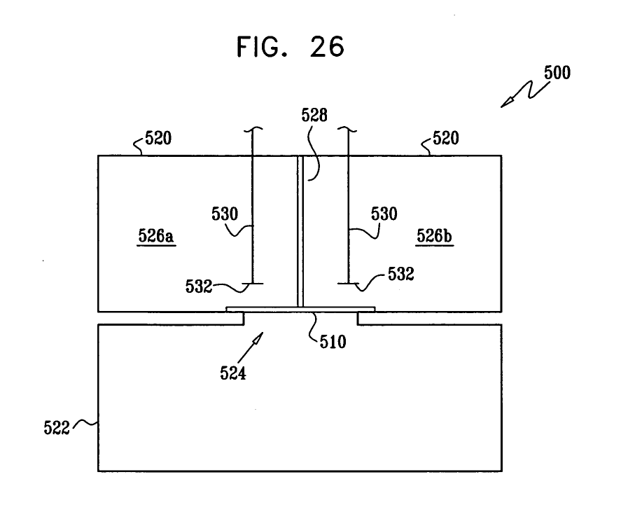

Reference is now made to FIG. 24, which is a graph showing in vitro experimental results measured in accordance with an embodiment of the present invention. A 300 g Wistar rat was anaesthetized using Ketamine (100 mg/kg) and Xylazine (10 mg/kg). Two 3 cm-long sections of the upper jejunum were removed and opened along the lumen so that two rectangular pieces of tissue were available. The serosal and muscular layers were removed using a microscope cover glass. The intestinal tissue segments were placed on slides and inserted into diffusion chambers similar to experimental diffusion chamber 500, described hereinbelow with reference to FIG. 26. Each diffusion chamber had a donor and an acceptor cell, connected by a 2.8 cm×8 mm window. The tissue segments on the slides completely covered the windows between the donor and acceptor cells. The cells were filled with 15 ml of Hank's Balanced Salt Solution (HBSS) (pH 7.4). The donor cells were then divided into two separate sections with a dividing board slightly touching the tissue so that fluid passage between the two parts of each donor cell was slow (if not impossible). The solution was maintained at 37° C. and gassed with 95% O₂/5% CO₂, supplied via 1 mm ID tubes placed at the bottom of each cell. Square stainless steel electrodes (316S, 6 mm×6 mm) were placed in the donor cells (one electrode in each section) in parallel with the tissue segments, at a 0.5 mm distance from the tissue. The distance between electrode centers was 10 mm. After 30 minutes in this state, the HBSS in the donor cells was replaced with 1 mg/ml octreotide acetate (Sandostatin) containing HBSS.

In one of the diffusion chambers (which served as a control), permeation of octreotide via the tissue segment was measured without the application of electrical stimulation. In the other diffusion chamber, a train of 12 Hz monophasic pulses 1 millisecond long were generated using a Thurlby Thandar Instruments TGP110 pulse generator. The voltage output of the pulse generator was adjusted so that a 3 mA current flowed through the electrodes. An EZ Digital Co. DM330 Digital Multimeter, connected serially to the electrodes was used to measure current. The multimeter was operating as a current meter, set to be sensitive to mA-level currents. One milliliter samples were taken from each of the acceptor cells 30 minutes after the pulse train start and every 15 minutes thereafter, over a 90-minute period. The samples were analyzed by HPLC-UV 205 nm spectroscopy (Hewlett-Packard 1100, acetonitril: phosphate buffer (pH 7.4) (40:60), C18 column) for their content of octreotide.

As can be seen in the graph of FIG. 24, a substantially greater increase in octreotide permeation occurred in the acceptor cell exposed to LITV pulses than occurred in the control acceptor cell. (Because octreotide acetate is not a charged molecule at the pH of the experiment, the inventors believe that iontophoresis was not responsible for the passage thereof between the chambers.)

As will be apparent to one of ordinary skill in the art having read the present patent application, it is also possible to configure capsule 102 to control the quantity of drug 106 administered. For example, drug 106 may be stored in several chambers within capsule 102, and the signal sent to the transmit/receive unit instructs the driving mechanism to deliver the drug from none, one, some, or all of the chambers.

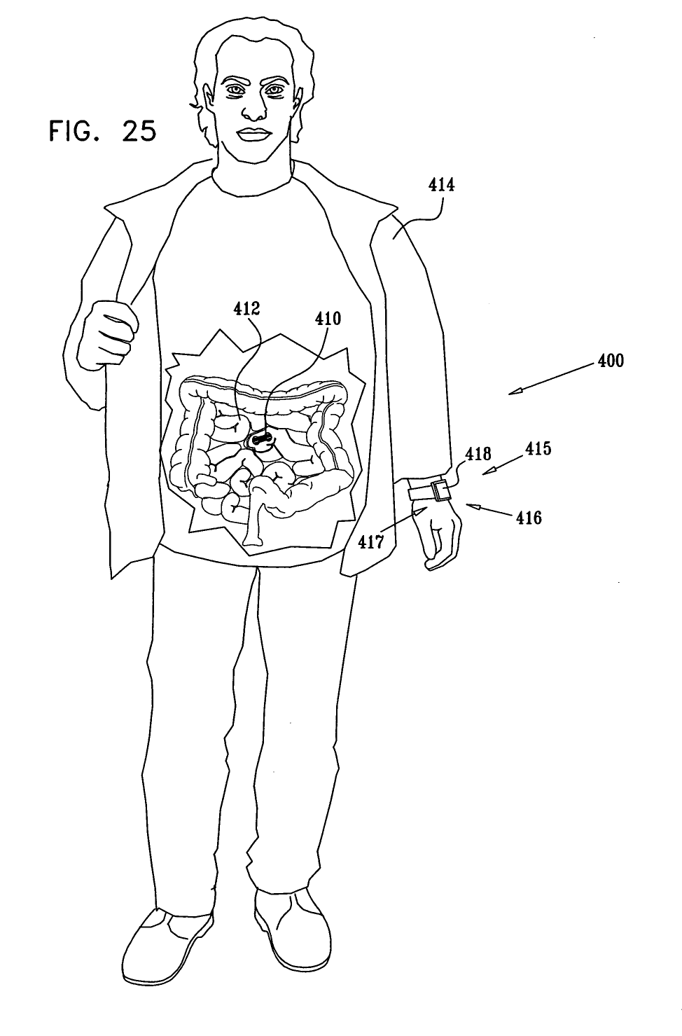

Reference is now made to FIG. 25, which is a schematic illustration of a closed-loop active drug-delivery system 400, in accordance with an embodiment of the present invention. System 400 comprises at least one ingestible drug-delivery device 410 (such as one of the ingestible drug-delivery devices described hereinabove), for facilitating passage of a drug through an epithelial layer of a GI tract 412 of a subject 414. System 400 further comprises a sensor unit 415, which comprises a sensor 416 coupled to a wireless transmitter 417, either wirelessly or over wires.

Sensor 416 is adapted to detect an indication of a concentration of the drug in the blood circulation of subject 414. For example, sensor 416 may comprise a noninvasive external sensor 418, e.g., a sensor adapted to be worn as a wristwatch. Noninvasive sensor 418 may, for example, utilize iontophoresis, infrared spectroscopy, or sonophoresis techniques for detecting the blood concentration of the drug, such as is known in the art for sensing blood glucose levels. Alternatively, sensor 416 comprises an invasive sensor, such as an implantable sensor, as is known in the art, e.g., for detecting blood glucose levels (configuration not shown).

Transmitter 417 is adapted to wirelessly transmit the detected indication to a receiver coupled to ingestible drug-delivery device 410 (receiver not shown). Drug-delivery device 410 is configured to adjust the level of facilitation of drug passage, responsively to the received indication, in order to regulate the level of the drug in the blood circulation. Device 410 typically increases the level of facilitation when the blood drug level is lower than a target value, and decreases the level of facilitation when the blood drug level is greater than a target value. Such closed-loop control of the blood drug level allows a physician to precisely prescribe the blood level of the drug, rather than only the dosage of the drug. For some applications, drug-delivery device 410 additionally comprises a transmitter, and sensor unit 415 additionally comprises a receiver. The drug-delivery device is adapted to wirelessly notify sensor unit 415 of the location of the drug-delivery device (e.g., the arrival of the device in the small intestine), the status of facilitation of transport, a pH of the GI tract, a temperature of the GI tract, and/or other operational parameters of the drug-delivery device.

In an embodiment of the present invention, ingestible drug-delivery device 410, in addition to facilitating the trans-epithelial passage of the drug through the epithelial layer, facilitates the trans-epithelial passage of a calibrating substance. Depending upon the specific type of drug-delivery device 410 employed, the calibrating substance is typically contained in the device, in a pill coupled to the device, or in a pill administered in conjunction with the device. (For some applications, the drug and the calibrating substance are contained in the same pill. Alternatively, for some applications, the drug and the calibrating substance are contained in separate pills.) Sensor unit 415 measures the level of the calibrating substance in the blood circulation, as a proxy for the level of the drug in the blood circulation. The use of the calibrating substance generally allows for standardization of the blood concentration detection techniques of sensor 416, and enables the use of drug-delivery system 400 even in cases in which the blood concentration of a particular drug is not readily detectable by sensor 416.

For some applications, sensor 416 is adapted to detect a level in the blood of a chemical (e.g., glucose), in response to which a dose of drug 106 (e.g., insulin) is administered or withheld by drug-delivery device 410. Alternatively or additionally, a parameter of the LITV signal or another applied signal is varied in response to the detected level. Suitable parameters include signal amplitude, a frequency of bursts (i.e., a number of bursts per time), an intra-burst pulse frequency, and/or a pulse width of applied pulses. Intermittently (for example, every minute or every ten minutes), sensor 416 performs another reading, and the operation of drug-delivery device 410 is regulated responsively to the updated reading. For other applications, instead of measuring the chemical glucose in order to modulate insulin administration, other chemical/drug pairs are utilized, such as the blood concentration of growth hormone and an administered growth hormone inhibitor (e.g., Sandostatin), as well as blood oxygenation as measured by a pulse oximetry unit in sensor 416 and a vasodilating administered drug.

In an embodiment, sensor 416 measures a non-chemical parameter, in order to facilitate suitable regulation of the operation of drug-delivery device 410. For example, sensor 416 may measure blood pressure, and drug 106 may comprise a diuretic. In this example, if blood pressure levels are normal, then diuretic administration is typically reduced or withheld. In another application, sensor 416 comprises a heart monitor (e.g., a pulse monitor or an ECG monitor). In yet another application, sensor 416 comprises an accelerometer and/or an indicator of a stage in the circadian cycle of subject 414 (e.g., timing circuitry), and the operation of drug-delivery device 410 is regulated responsive thereto. For example, drug-delivery device 410 may increase administration of an antithrombotic drug (e.g., low molecular weight Heparin) during the day, and decrease administration thereof at night. In another application, sensor 416 comprises a temperature sensor, and drug 106 comprises an antibiotic (e.g., cefazolin).

With respect to each of the uses of drug-delivery system 400, it is noted that for some applications, subject 414 may swallow a capsule according to a schedule, but generally regardless of a current need for the drug. If a need arises, the drug is delivered, typically at a dose that is regulated in real time (i.e., while the capsule is in the subject's body). If no need arises, then no drug is administered.

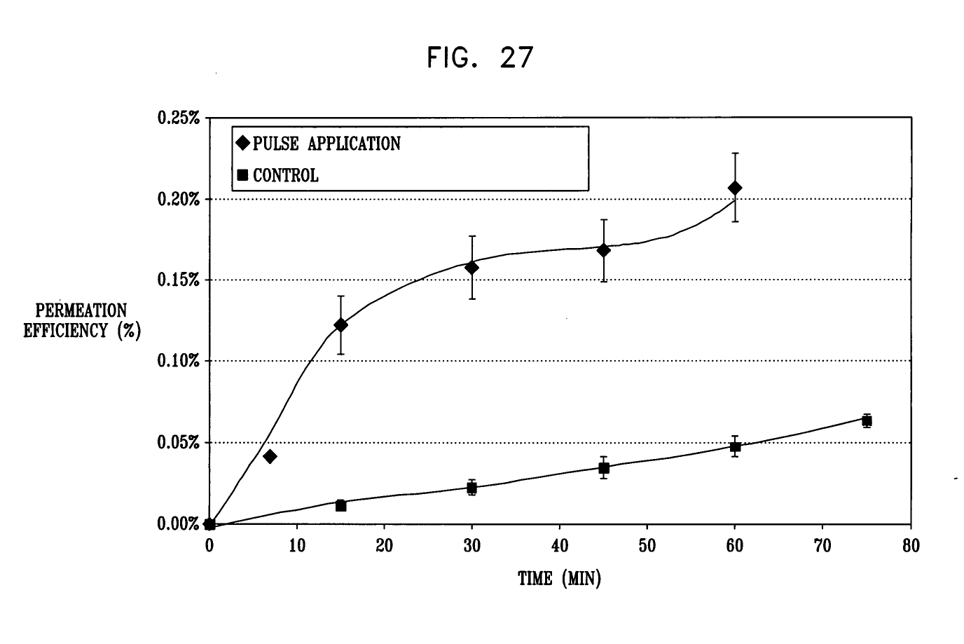

Reference is now made to FIG. 26, which is a schematic cross-sectional illustration of an experimental diffusion chamber 500, and FIGS. 27-36, which are graphs showing in vitro experimental results generated in accordance with respective embodiments of the present invention. A number of 300 g Wistar rats were anaesthetized using Ketamine (100 mg/kg). and Xylazine (10 mg/kg). Two 3 cm-long sections 510 of the intestine were removed from each rat and opened along the mesenterial line so that two rectangular pieces of tissue were available from each rat (a single tissue section 510 is shown in FIG. 26). For the experiments described hereinbelow with reference to FIGS. 27-35, the intestinal sections were taken from the upper jejunum, while for the experiment described hereinbelow with reference to FIG. 36, the intestinal sections were taken from the upper jejunum, proximal ileum, and distal ileum. The serosal and muscular layers of the intestinal sections were removed using a microscope cover glass. Each of the intestinal tissue segments was placed on a slide and inserted into diffusion chamber 500.

Diffusion chamber 500 is shaped so as to define a donor cell 520 and an acceptor cell 522, connected by a 28 mm×8 mm window 524. Tissue segment 510 on the slide completely covered window 524. Tissue segment 510 was placed so as to completely cover window 524, thereby separating donor cell 520 and acceptor cell 522. Tissue segment 510 was oriented such that the mucosal side thereof faced donor cell 520, and the serosal side thereof faced acceptor cell 522. Donor cell 520 was filled with 15 ml of Hank's Balanced Salt Solution (HBSS) adjusted to a pH of 7.4 (in mM: 136.9 NaCl, 5.4 KCl, 0.5 MgCl₂, 0.4 MgSO₄, 4.5 KH₂PO₄, 0.35 Na₂HPO₄, 1.0 CaCl₂, 4.2 NaHCO₃, 5.5 D-Glucose). Acceptor cell 522 was filled with D-Glucose-supplemented Phosphate Buffered Saline (PBS) adjusted to a pH of 7.4 (in mM: 136.9 NaCl, 2.7 KCl, 0.5 MgCl₂, 1.5 KH₂PO₄, 8.1 Na₂HPO₄, 0.7 CaCl₂, 5.5 D-Glucose).

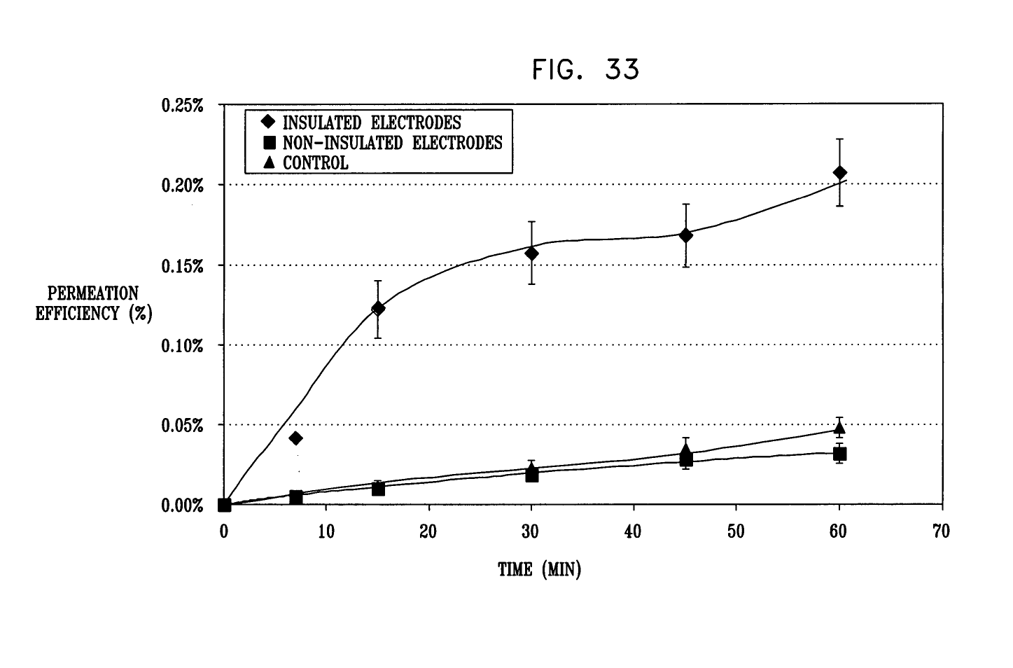

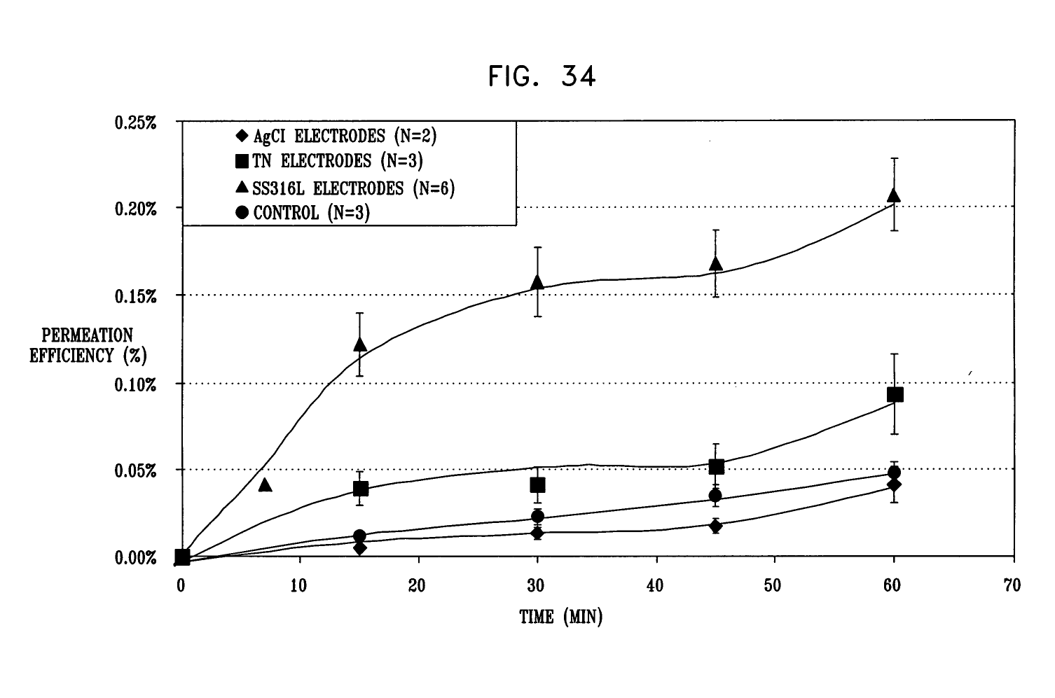

After tissue segment 510 was placed over window 524, the donor cell was divided into two separate compartments 526a and 526b by an electrically-insulating divider 528 positioned to slightly touch tissue segment 510 so that fluid passage between compartments 526a and 526b was slow (if not impossible). (Donor cell 520 was not divided into compartments 526a and 526b in the experiment described hereinbelow with reference to FIG. 33.) The solution was maintained at 37° C. and gassed with 95% O₂/5% CO₂, supplied via 1 mm ID tubes placed at the bottom of each cell (tubes not shown in FIG. 26).

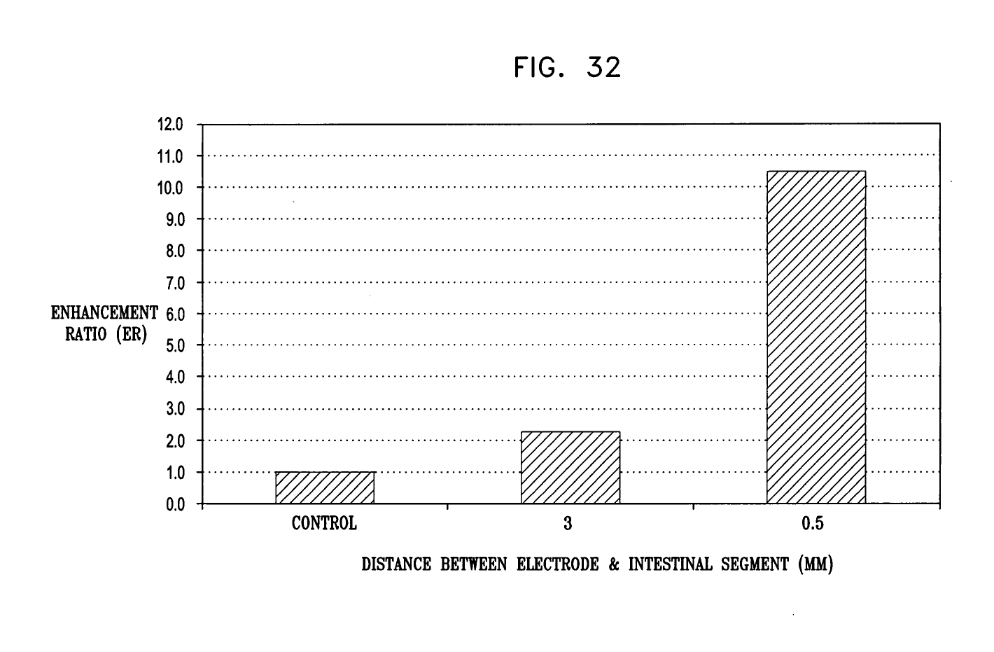

A single square electrode 530 was placed in each of compartments 526a and 526b of donor cell 520, such that an electrode surface 532 of each electrode was parallel to the surface of tissue segment 510, at a 0.5 mm distance from tissue segment 510 (except for the experiment described hereinbelow with reference to FIG. 32). Electrodes 530 comprised stainless steel (SS316L, 6 mm×6 mm) (except for the experiment described hereinbelow with reference to FIG. 34). The distance between the centers of electrode surfaces 532 was 10 mm. After tissue segment 510 was in position over window 524 for 30 minutes, the HBSS in donor cell 520 was replaced with 1 mg/ml octreotide acetate (Sandostatin) containing HBSS.

In each of the experiments described hereinbelow with reference to FIGS. 27-36, beginning upon replacement of the HBSS in donor cell 520 with octreotide, a train of LITV pulses was applied through electrodes 530, and the permeation of octreotide from donor cell 520 to acceptor cell 522 via tissue segment 510 was measured. This train of monophasic rectangular pulses was generated using a Thurlby Thandar Instruments TGP110 pulse generator. The voltage output of the pulse generator was adjusted so that a 3 mA current flowed through the electrodes. An EZ Digital Co. DM330 Digital Multimeter, connected serially to the electrodes, was used to measure current. The multimeter was operating as a current meter, set to be sensitive to mA-level currents.

One milliliter samples of the incubation medium were taken from acceptor cell 522 at 7 minutes and 14 minutes after replacement of the HBSS with octreotide, and every 15 minutes thereafter, over a 90-minute period. The samples were analyzed for their content of octreotide by HPLC-UV 205 nm spectroscopy (Hewlett-Packard 1100). Isocratic elution was performed with a phosphate buffer (pH 7.4) and acetonitril as a mobile phase (40:60 w/w), at a flow rate of 1.2 ml/minute. A 100×3 mm C18 column was used.

For each of the experiments, at least two tissue segments from different rats served as the experimental group or groups (no single rat donated more than one tissue segment to any experimental group of any of the experiments). Each tissue segment was separately placed in diffusion chamber 500, electrical pulses were applied, and permeation of octreotide via the tissue segment was measured. In addition, for each of the experiments, at least two (generally three) tissue segments from different rats served as a control group (no single rat donated more than one tissue segment to the control group of any of the experiments). The tissue segments of the control groups were separately placed in diffusion chamber 500, and permeation of octreotide via the tissue segments was measured without the application of an electrical signal.

For the experiments described hereinbelow with reference to FIGS. 27-36, the effectiveness of the application of the electrical signal is expressed as permeation efficiency (PE), which is defined as the ratio of (a) the amount of octreotide permeated via tissue section 510 to (b) the initial amount of octreotide in donor cell 520 of diffusion chamber 500, as defined by the following equation:
PE(%)=dQ/Qi×100%,
where dQ represents the amount of octreotide that has entered acceptor cell 522 of chamber 500 up to a given point in time, and Qᵢ represents the initial amount of octreotide administered to donor cell 520 of chamber 500.

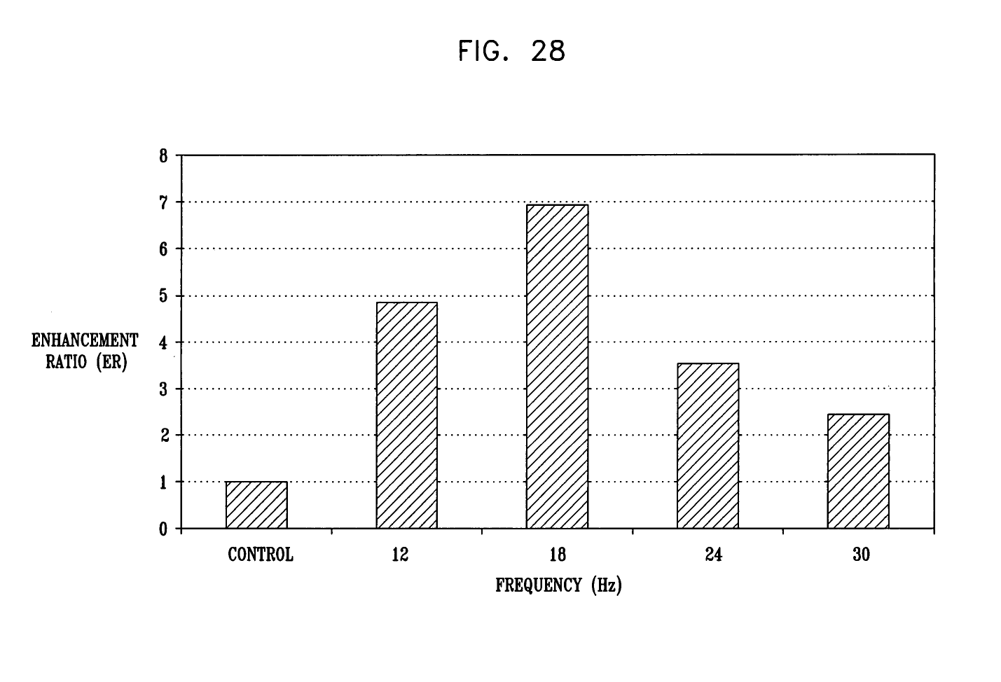

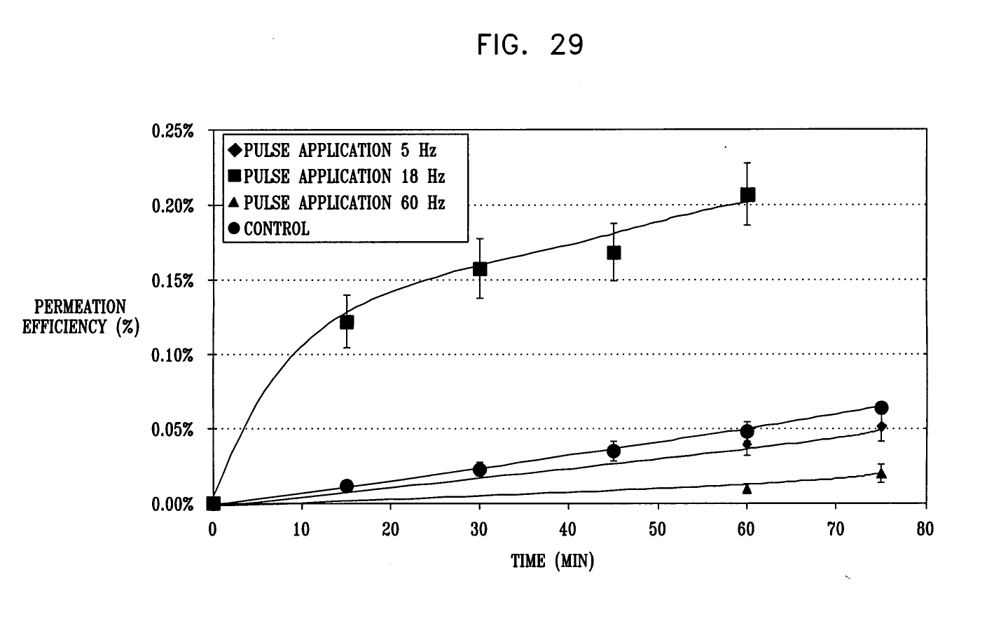

For the experiments described hereinbelow with reference to FIGS. 28, 30, and 32, the effectiveness of the application of the electrical signal is expressed as a transport enhancement ratio (ER), which is defined as the ratio of (a) the PE measured during signal application in the experimental group to (b) the PE measured in the control group.

Reference is made to FIG. 27, which is a graph showing the effect of electrical signal application on permeation efficiency, generated in accordance with an embodiment of the present invention. Monophasic rectangular pulses were applied to 6 jejunal tissue samples taken from 6 different rats, while 3 jejunal tissue samples taken from 3 different rats served as a control group. (The data from these experimental and control groups were also used in the experiments described hereinbelow with reference to FIGS. 28-36.) The pulses had a pulse duration of 1 millisecond, a frequency of 18 Hz, and a strength of 3 mA. As can be seen in the graph, application of the pulses substantially enhanced octreotide permeation compared with octreotide permeation in the non-stimulated control group.

FIGS. 28 and 29 are graphs showing the effect of pulse frequency on permeation efficiency, generated in accordance with an embodiment of the present invention. Monophasic rectangular pulses were applied to 15 jejunal tissue samples to generate the data shown in FIG. 28, and to 8 jejunal tissue samples to generate the data shown in FIG. 29. As mentioned above, the control group of FIG. 27 was used as the control group. The pulses had a pulse duration of 1 millisecond and a strength of 3 mA. Several pulse frequencies were tested (5 Hz (n=1), 12 Hz (n=5), 18 Hz (n=6), 24 Hz (n=2), 30 Hz (n=2), and 60 Hz (n=1)). (For the 18 Hz experimental group, the experimental group of FIG. 27 was used.) As can be seen in the graph of FIG. 28, at 30 minutes after replacement of the HBSS with octreotide, application of the pulses at 18 Hz achieved the greatest enhancement ratio. As can be seen in the graph of FIG. 29, application of the pulses at 5 Hz and 60 Hz did not yield a higher octreotide permeation than the octreotide permeation in the control group.

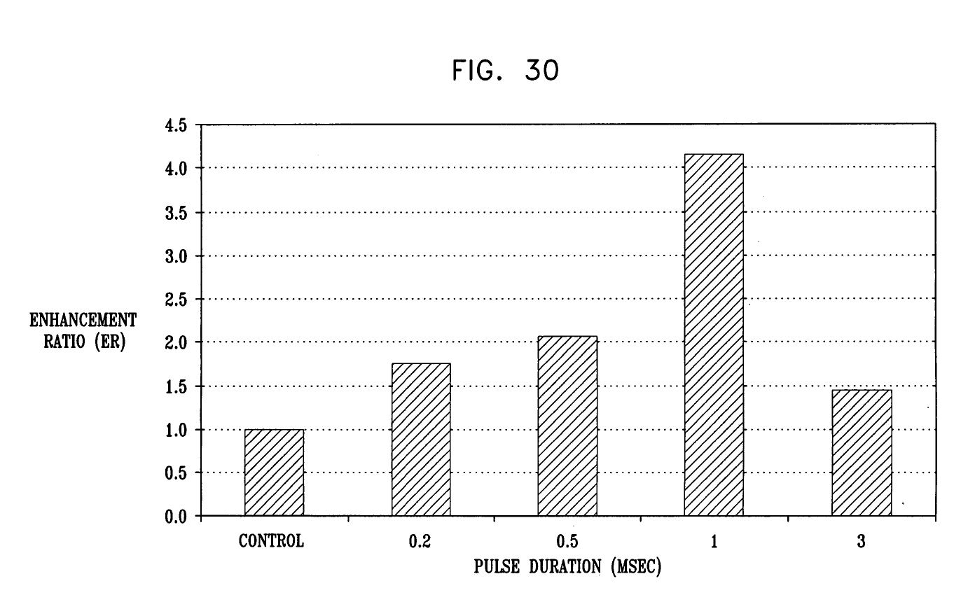

FIG. 30 is a graph showing the effect of pulse duration on permeation efficiency, generated in accordance with an embodiment of the present invention. Monophasic rectangular pulses were applied to 13 jejunal tissue samples, and the control group of FIG. 27 was used as the control group. The pulses had a frequency of 18 Hz and a strength of 3 mA. Several pulse durations were tested (0.2 milliseconds (n=2), 0.5 milliseconds (n=3), 1 millisecond (n=6), and 3 milliseconds (n=2)). (For the 1 millisecond experimental group, the experimental group of FIG. 27 was used.) As can be seen in the graph, at 15 minutes after replacement of the HBSS with octreotide, application of the pulses with a pulse duration of 1 millisecond achieved the greatest enhancement ratio.

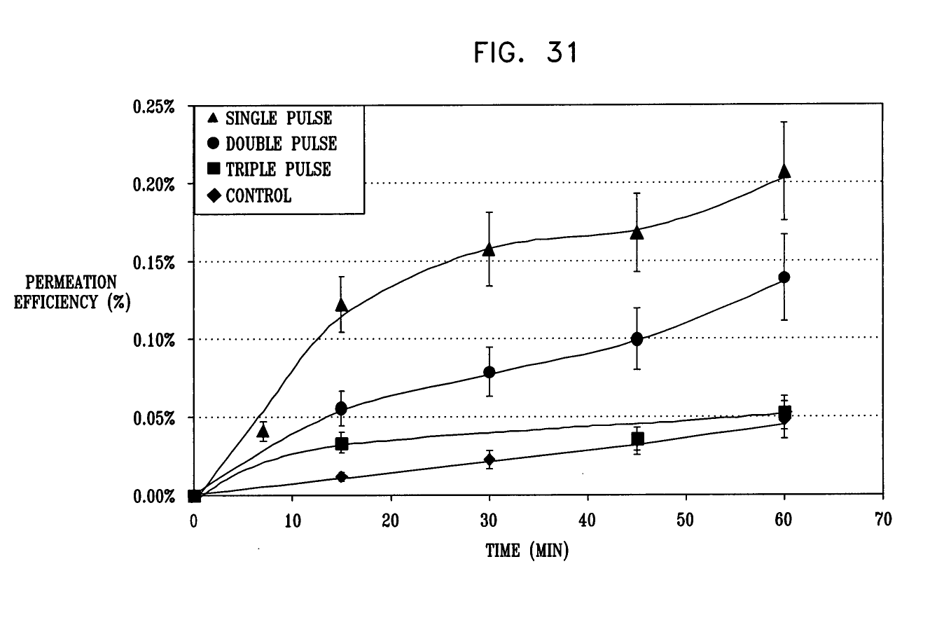

FIG. 31 is a graph showing the effect of pulse cycle on permeation efficiency, generated in accordance with an embodiment of the present invention. Monophasic rectangular pulses were applied to 10 jejunal tissue samples, and the control group of FIG. 27 was used as the control group. The pulses had a frequency of 18 Hz, a strength of 3 mA, and a pulse duration of 1 millisecond. Several pulse cycles (i.e., number of pulses per pulse application within the train of pulses) were tested (1 pulse per cycle (n=6); 2 pulses per cycle, with the second pulse commencing 5 milliseconds after commencement of the first pulse (n=2); and 3 pulses per cycle, with successive pulses commencing at 5-millisecond intervals (n=2)). (For the 1 pulse per cycle experimental group, the experimental group of FIG. 27 was used.) As can be seen in the graph, as the number of pulses per cycle increased, the permeation efficiency decreased, such that the greatest permeation efficiency was achieved at 1 pulse per cycle.

FIG. 32 is a graph showing the effect of electrode distance from jejunal tissue on permeation efficiency, generated in accordance with an embodiment of the present invention. Monophasic rectangular pulses were applied to 8 jejunal tissue samples, and the control group of FIG. 27 was used as the control group. The pulses had a frequency of 18 Hz, a strength of 3 mA, and a pulse duration of 1 millisecond. The pulses were applied at two electrode distances from the jejunal tissue, 0.5 mm (n=2) and 3 mm (n=6). (For the 3 mm experimental group, the experimental group of FIG. 27 was used.) As can be seen in the graph, at 15 minutes after replacement of the HBSS with octreotide, the magnitude of permeation efficiency was greater at 0.5 mm than at 3 mm from the jejunal tissue.

FIG. 33 is a graph showing the effect of electrode insulation on permeation efficiency, generated in accordance with an embodiment of the present invention. Monophasic rectangular pulses were applied to 7 jejunal tissue samples, and the control group of FIG. 27 was used as the control group. The pulses had a frequency of 18 Hz, a strength of 3 mA, and a pulse duration of 1 millisecond. The pulses were applied both with divider 528 (FIG. 26), which provided electrical insulation between the two electrodes (the experimental group of FIG. 27 was used (n=6)), and without divider 528, such that the electrodes were not electrically insulated from each other (n=1). As can be seen in the graph, application of the pulses did not increase permeation efficiency when the electrodes were not insulated from each other by divider 528.

FIG. 34 is a graph showing the effect of electrode material on permeation efficiency, generated in accordance with an embodiment of the present invention. Monophasic rectangular pulses were applied to 11 jejunal tissue samples, and the control group of FIG. 27 was used as the control group. The pulses had a frequency of 18 Hz, a strength of 3 mA, and a pulse duration of 1 millisecond. The pulses were applied using stainless steel (SS316L) electrodes (n=6), titanium nitride (TN) electrodes (n=3), and silver chloride (AgCl) electrodes (n=2). (For the stainless steel electrodes experimental group, the experimental group of FIG. 27 was used.) As can be seen in the graph, application of the pulses using stainless steel electrodes substantially increased permeation efficiency, while application of the pulses with titanium nitride electrodes and silver chloride electrodes did not increase permeation efficiency.

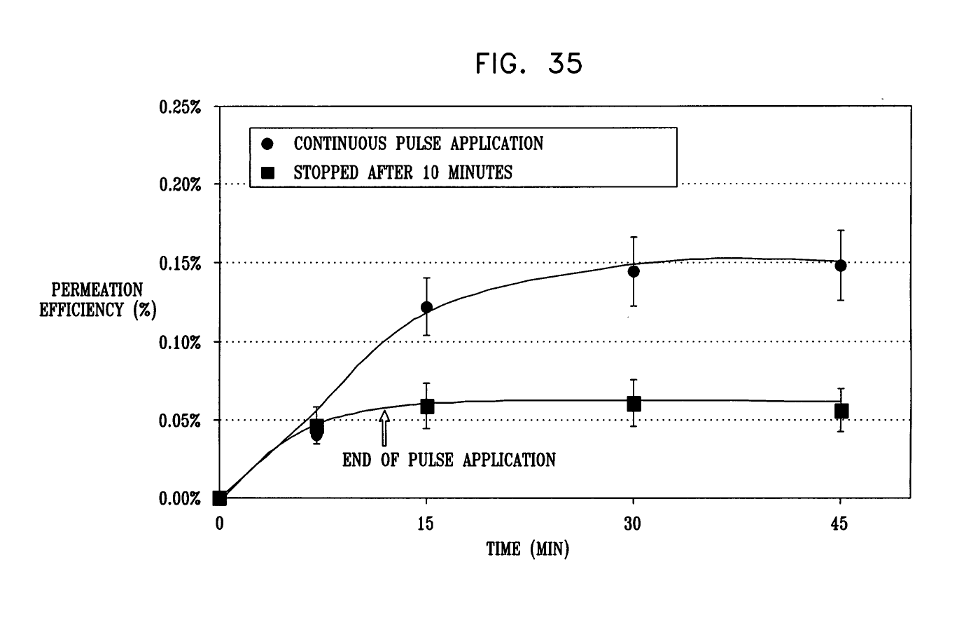

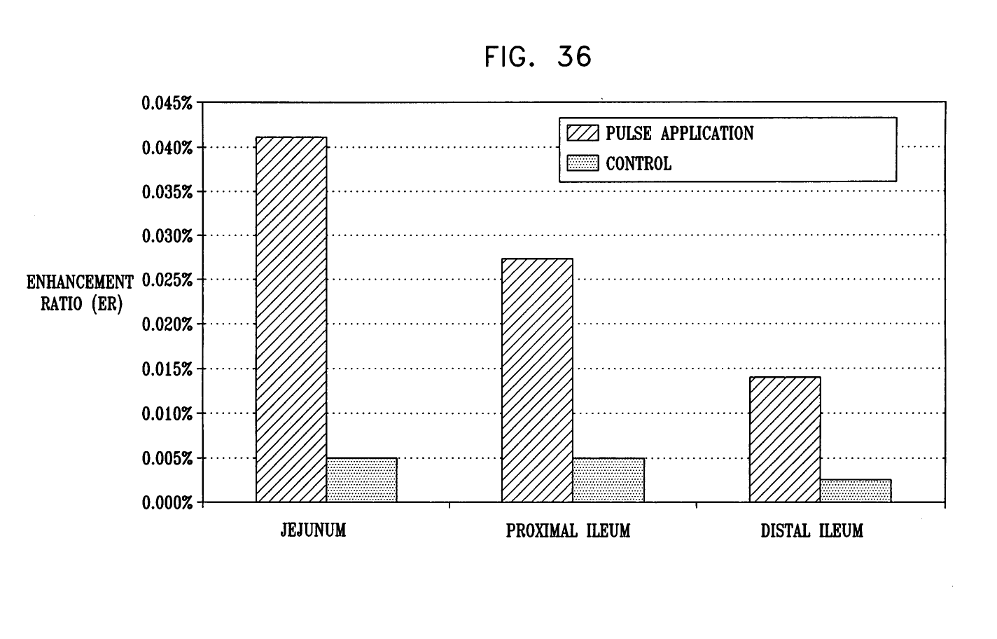

FIG. 35 is a graph showing the effect of cessation of pulse application on permeation efficiency, generated in accordance with an embodiment of the present invention. Monophasic rectangular pulses were applied to 7 jejunal tissue samples. The experimental group included one tissue sample, for which pulse application was stopped after 10 minutes of application. The experimental group described hereinabove with reference to FIG. 27 served as the control group; pulses were applied to this control group continuously throughout the experimental period (for a total of 60 minutes, 45 minutes of which are shown in FIG. 35). The pulses applied to both the experimental group and the control group had a frequency of 18 Hz, a strength of 3 mA, and a pulse duration of 1 millisecond. As can be seen in the graph (which is normalized to the octreotide permeation of the control group of FIG. 27), continuous application of the pulses resulted in substantially greater permeation efficiency compared to cessation of application of the pulses after 10 minutes.

FIG. 36 is a graph showing permeation efficiency in different regions of the intestine, generated in accordance with an embodiment of the present invention. Monophasic rectangular pulses were applied to 6 jejunal tissue samples (the experimental group of FIG. 27 was used), 2 proximal ileum tissue samples, and 2 distal ileum tissue samples. Three jejunal tissue samples (the control group of FIG. 27 was used), 2 proximal ileum tissue samples, and 3 distal ileum tissue samples served as control groups. The pulses had a frequency of 18 Hz, a strength of 3 mA, and a pulse duration of 1 millisecond. As can be seen in the graph, at 7 minutes after replacement of the HBSS with octreotide, pulse application to tissue from all three of the intestinal regions increased permeation efficiency, with the greatest effect of pulse application in the jejunal tissue samples, and a positive but less pronounced effect in the distal ileum tissue samples.

Although the parameters in these experiments were applied to rats, the inventors believe that similar parameters are appropriate for application to human subjects, given relevant physiological similarities between rats and humans.

For some applications, techniques described hereinabove are practiced in combination with techniques described in one or more of the articles, patents and/or patent applications mentioned hereinabove. By way of example and not limitation, embodiments of the present invention comprising a piston or spring may use spring-release techniques described in one or more of these patents or patent applications.

It is expected that during the life of this patent many relevant drugs will be developed and the scope of the term drug is intended to include all such new technologies a priori.

As used herein the term “about” refers to ±10%.

In the description hereinabove of embodiments of the invention, various oral dosage forms are described, for example, capsules and tablets. In the claims, the word “capsule” is to be understood to refer to oral dosage forms generally, i.e., comprising capsules, tablets, and similar forms, for example, as shown in FIGS. 3-20 with respect to drug-delivery system 30, or as shown in FIGS. 21-30 with respect to capsule 102.

As used in the context of the present patent application and in the claims, the word “drug” means any natural or synthetic chemical that may be administered as an aid in the diagnosis, treatment, cure, mitigation, or prevention of disease or other abnormal conditions, or to improve health.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

As appropriate, techniques described in the present patent application may be practiced in combination with techniques described in a U.S. regular patent application and a PCT patent application, both entitled, “Active drug delivery in the gastrointestinal tract,” filed on Jan. 29, 2004, incorporated herein by reference, and assigned to the assignee of the present patent application.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. Apparatus for drug administration, comprising an ingestible capsule, which comprises:
a drug, stored by the capsule;
an environmentally-sensitive mechanism, adapted to change a state thereof responsively to a disposition of the capsule within a gastrointestinal (GI) tract of a subject;
first and second electrodes; and
a control component, adapted to facilitate passage of the drug, in response to a change of state of the environmentally-sensitive mechanism, through an epithelial layer of the GI tract by driving the first and second electrodes to apply a series of pulses at a current of less than about 5 mA, at a frequency of between about 12 Hz and about 24 Hz, and with a pulse duration of between about 0.5 milliseconds and about 3 milliseconds.

2. The apparatus according to claim 1, wherein the pulses include monophasic rectangular pulses, and wherein the control component is adapted to drive the first and second electrodes to apply the series of monophasic rectangular pulses.

3. The apparatus according to claim 1, wherein the first and second electrodes comprise stainless steel.

4. The apparatus according to claim 1,
wherein the environmentally-sensitive mechanism comprises a sensor adapted to sense an indication of a distance traveled by the capsule in the GI tract, and
wherein the environmentally-sensitive mechanism is adapted to undergo the change of state responsive to the distance.

5. The apparatus according to claim 1, wherein the environmentally-sensitive mechanism comprises a camera, adapted to image the GI tract, and wherein the control component is adapted to drive the first and second electrodes to apply the series of pulses in response to an image acquired by the camera.

6. The apparatus according to claim 1, wherein the disposition of the capsule includes a temperature in a vicinity of the capsule, wherein the environmentally-sensitive mechanism comprises a temperature sensor, and wherein the control component is adapted to drive the first and second electrodes to apply the series of pulses in response to the temperature sensed by the temperature sensor.

7. The apparatus according to claim 1, wherein the disposition of the capsule includes a pH in a vicinity of the capsule, wherein the environmentally-sensitive mechanism comprises a pH sensor, and wherein the control component is adapted to drive the first and second electrodes to apply the series of pulses in response to the pH sensed by the pH sensor.

8. The apparatus according to claim 1, wherein the environmentally-sensitive mechanism comprises a sensor, adapted to sense a characteristic of the GI tract, and wherein the control component is adapted to drive the first and second electrodes to apply the series of pulses in response to the sensed characteristic.

9. The apparatus according to claim 1, wherein the control component is adapted to:
drive the first and second electrodes to apply the series of pulses, and
drive an iontophoretic current between the first and second electrodes.

10. The apparatus according to claim 1, wherein the control component is adapted to configure the series of pulses using parameters selected at least in part responsively to the disposition of the capsule within the GI tract.

11. The apparatus according to claim 1, wherein the control component is adapted to configure the series of pulses using parameters selected at least in part responsively to a property of the drug.

12. The apparatus according to claim 1, wherein the capsule comprises a central portion, intermediate the first and second electrodes, a shape of the central portion being such as to reduce current flow within a lumen of the GI tract.

13. The apparatus according to claim 1, wherein the capsule comprises a central portion, intermediate the first and second electrodes, the central portion having a diameter that is such as to bring the central portion in contact with the epithelial layer of the GI tract, whereby to reduce current flow within a lumen of the GI tract.

14. The apparatus according to claim 1, wherein the capsule comprises a self-expansible central portion, intermediate the first and second electrodes, the central portion adapted to expand, in response to being in the GI tract, to have a diameter that is such as to bring the central portion in contact with the epithelial layer of the GI tract, whereby to reduce current flow within a lumen of the GI tract.

15. The apparatus according to claim 1, wherein the capsule comprises a central portion, intermediate the first and second electrodes, an outer surface of the central portion comprising a hydrophobic material.

16. The apparatus according to claim 1, wherein the capsule comprises a central portion, intermediate the first and second electrodes, an outer surface of the central portion comprising a lipophilic material.

17. The apparatus according to claim 1, wherein the environmentally-sensitive mechanism is essentially entirely biodegradable.

18. The apparatus according to claim 1, wherein the first and second electrodes and the control component are essentially entirely biodegradable.

19. The apparatus according to claim 1, wherein at least 80% of the mass of the capsule is biodegradable.

20. The apparatus according to claim 19, wherein at least 95% of the mass of the capsule is biodegradable.

21. The apparatus according to-claim 20, wherein essentially the entire capsule is biodegradable.

22. The apparatus according to claim 1, wherein the environmentally-sensitive mechanism comprises a coating on a surface of the capsule.

23. The apparatus according to claim 22, wherein the coating comprises a pH-sensitive coating.

24. The apparatus according to claim 1, wherein the control component is adapted to apply the series of pulses at a current of between about 2 mA and about 4 mA.

25. The apparatus according to claim 24, wherein the control component is adapted to drive the first and second electrodes to apply the series of pulses at a current of about 3 mA.

26. The apparatus according to claim 1, wherein the control component is adapted to drive the first and second electrodes to apply the series of pulses at a frequency of between about 16 Hz and about 20 Hz.

27. The apparatus according to claim 26, wherein the control component is adapted to drive the first and second electrodes to apply the series of pulses at a frequency of about 18 Hz.

28. The apparatus according to claim 1, wherein the control component is adapted to drive the first and second electrodes to apply the series of pulses with a pulse duration of between about 0.5 milliseconds and about 1.5 milliseconds.

29. The apparatus according to claim 28, wherein the control component is adapted to drive the first and second electrodes to apply the series of pulses with a pulse duration of about 1 millisecond.

30. The apparatus according to claim 1, wherein the control component is adapted to drive the first and second electrodes to apply the series of pulses for a period of between about 1 and about 360 minutes.

31. The apparatus according to claim 30, wherein the control component is adapted to drive the first and second electrodes to apply the series of pulses for a period of between about 60 and about 240 minutes.

32. Apparatus for administration of a drug, comprising an ingestible capsule adapted to store the drug, the capsule comprising:
an environmentally-sensitive mechanism, adapted to change a state thereof responsively to a disposition of the capsule within a gastrointestinal (GI) tract of a subject;
first and second electrodes; and
a control component, adapted to facilitate passage of the drug, in response to a change of state of the environmentally-sensitive mechanism, through an epithelial layer of the GI tract by driving the first and second electrodes to apply a series of pulses at a current of less than about 5 mA, at a frequency of between about 12 Hz and about 24 Hz, and with a pulse duration of between about 0.5 milliseconds and about 3 milliseconds.

33. The apparatus according to claim 32, wherein the control component is adapted to apply the series of pulses at a current of between about 2 mA and about 4 mA.

34. The apparatus according to claim 33, wherein the control component is adapted to drive the first and second electrodes to apply the series of pulses at a current of about 3 mA.

35. The apparatus according to claim 32, wherein the control component is adapted to drive the first and second electrodes to apply the series of pulses at a frequency of between about 16 Hz and about 20 Hz.

36. The apparatus according to claim 35, wherein the control component is adapted to drive the first and second electrodes to apply the series of pulses at a frequency of about 18 Hz.

37. The apparatus according to claim 32, wherein the control component is adapted to drive the first and second electrodes to apply the series of pulses with a pulse duration of between about 0.5 milliseconds and about 1.5 milliseconds.

38. The apparatus according to claim 37, wherein the control component is adapted to drive the first and second electrodes to apply the series of pulses with a pulse duration of about 1 millisecond.

39. The apparatus according to claim 32, wherein the control component is adapted to drive the first and second electrodes to apply the series of pulses for a period of between about 1 and about 360 minutes.

40. The apparatus according to claim 39, wherein the control component is adapted to drive the first and second electrodes to apply the series of pulses for a period of between about 60 and about 240 minutes.

41. Apparatus for facilitating administration of a drug contained in a pill, the apparatus comprising an ingestible housing, which is not adapted to contain the drug or to be assembled in an integral unit with the drug, the housing comprising:
an ingestible environmentally-sensitive mechanism, adapted to change a state thereof responsive to a disposition thereof within a gastrointestinal (GI) tract of a subject;
first and second electrodes; and
a control component, adapted to facilitate passage of the drug, in response to a change of state of the environmentally-sensitive mechanism, through an epithelial layer of the GI tract by driving the first and second electrodes to apply a series of pulses at a current of less than about 5 mA, at a frequency of between about 12 Hz and about 24 Hz, and with a pulse duration of between about 0.5 milliseconds and about 3 milliseconds.

42. The apparatus according to claim 41,
wherein the environmentally-sensitive mechanism comprises a sensor adapted to sense an indication of a distance traveled by the housing in the GI tract, and
wherein the environmentally-sensitive mechanism is adapted to undergo the change of state responsive to the distance.

43. The apparatus according to claim 41, wherein the environmentally-sensitive mechanism comprises a camera, adapted to image the GI tract, and wherein the control component is adapted to drive the first and second electrodes to apply the series of pulses in response to an image acquired by the camera.

44. The apparatus according to claim 41, wherein the disposition of the environmentally-sensitive mechanism includes a temperature in a vicinity of the environmentally-sensitive mechanism, wherein the environmentally-sensitive mechanism comprises a temperature sensor, and wherein the control component is adapted to drive the first and second electrodes to apply the series of pulses in response to the temperature sensed by the temperature sensor.

45. The apparatus according to claim 41, wherein the disposition of the environmentally-sensitive mechanism includes a pH in a vicinity of the environmentally-sensitive mechanism, wherein the environmentally-sensitive mechanism comprises a pH sensor, and wherein the control component is adapted to drive the first and second electrodes to apply the series of pulses in response to the pH sensed by the pH sensor.

46. The apparatus according to claim 41, wherein the environmentally-sensitive mechanism comprises a sensor, adapted to sense a characteristic of the GI tract, and wherein the control component is adapted to drive the first and second electrodes to apply the series of pulses in response to the sensed characteristic.

47. The apparatus according to claim 41, wherein the environmentally-sensitive mechanism is adapted to undergo the change of state generally at an expected time of release of the drug from the drug pill.

48. The apparatus according to claim 41, wherein the environmentally-sensitive mechanism comprises a coating on a surface of the housing.

49. The apparatus according to claim 48, wherein the coating comprises a pH-sensitive coating.

50. The apparatus according to claim 41, wherein the control component is adapted to apply the series of pulses at a current of between about 2 mA and about 4 mA.

51. The apparatus according to claim 50, wherein the control component is adapted to drive the first and second electrodes to apply the series of pulses at a current of about 3 mA.

52. The apparatus according to claim 41, wherein the control component is adapted to drive the first and second electrodes to apply the series of pulses at a frequency of between about 16 Hz and about 20 Hz.

53. The apparatus according to claim 52, wherein the control component is adapted to drive the first and second electrodes to apply the series of pulses at a frequency of about 18 Hz.

54. The apparatus according to claim 41, wherein the control component is adapted to drive the first and second electrodes to apply the series of pulses with a pulse duration of between about 0.5 milliseconds and about 1.5 milliseconds.

55. The apparatus according to claim 54, wherein the control component is adapted to drive the first and second electrodes to apply the series of pulses with a pulse duration of about 1 millisecond.

56. The apparatus according to claim 41, wherein the control component is adapted to drive the first and second electrodes to apply the series of pulses for a period of between about 1 and about 360 minutes.

57. The apparatus according to claim 56, wherein the control component is adapted to drive the first and second electrodes to apply the series of pulses for a period of between about 60 and about 240 minutes.

58. Apparatus for use with a drug pill, the apparatus comprising:
a coupling mechanism, adapted to couple the drug pill to the apparatus;
first and second electrodes; and
a control component, adapted to facilitate passage of a drug contained in the drug pill through an epithelial layer of a gastrointestinal (GI) tract of a subject by driving the first and second electrodes to apply a series of pulses at a current of less than about 5 mA, at a frequency of between about 12 Hz and about 24 Hz, and with a pulse duration of between about 0.5 milliseconds and about 3 milliseconds.

59. The apparatus according to claim 58, wherein the drug pill includes a commercially-available drug pill, and wherein the coupling mechanism is adapted to couple the commercially-available drug pill to the apparatus.

60. The apparatus according to claim 58, wherein the coupling mechanism comprises an adhesive.

61. The apparatus according to claim 58, wherein the coupling mechanism comprises at least one of the electrodes.

62. The apparatus according to claim 61, wherein the at least one of the electrodes is configured to surround a portion of the drug pill once the drug pill has been coupled to the apparatus.

63. The apparatus according to claim 58, wherein the control component is adapted to apply the series of pulses at a current of between about 2 mA and about 4 mA.

64. The apparatus according to claim 63, wherein the control component is adapted to drive the first and second electrodes to apply the series of pulses at a current of about 3 mA.

65. The apparatus according to claim 58, wherein the control component is adapted to drive the first and second electrodes to apply the series of pulses at a frequency of between about 16 Hz and about 20 Hz.

66. The apparatus according to claim 65, wherein the control component is adapted to drive the first and second electrodes to apply the series of pulses at a frequency of about 18 Hz.

67. The apparatus according to claim 58, wherein the control component is adapted to drive the first and second electrodes to apply the series of pulses with a pulse duration of between about 0.5 milliseconds and about 1.5 milliseconds.

68. The apparatus according to claim 67, wherein the control component is adapted to drive the first and second electrodes to apply the series of pulses with a pulse duration of about 1 millisecond.

69. The apparatus according to claim 58, wherein the control component is adapted to drive the first and second electrodes to apply the series of pulses for a period of between about 1 and about 360 minutes.

70. The apparatus according to claim 69, wherein the control component is adapted to drive the first and second electrodes to apply the series of pulses for a period of between about 60 and about 240 minutes.

71. Apparatus for facilitating administration of a drug to a subject, the apparatus comprising:
a sensor unit, which comprises:
a sensor, adapted to detect an indication of a concentration of a substance in a blood circulation of the subject; and
a wireless transmitter, adapted to wirelessly transmit the indication; and
an ingestible capsule, which comprises:
a wireless receiver, adapted to receive the indication;
first and second electrodes; and
a control component, adapted to facilitate passage of the drug through an epithelial layer of a gastrointestinal (GI) tract of the subject by driving the first and second electrodes to apply a series of pulses at a current of less than about 5 mA, at a frequency of between about 12 Hz and about 24 Hz, and with a pulse duration of between about 0.5 milliseconds and about 3 milliseconds.

72. The apparatus according to claim 71, wherein the substance includes the drug, and wherein the sensor is adapted to detect the indication of the concentration of the drug in the blood circulation.

73. The apparatus according to claim 71,
wherein the substance includes a calibrating substance,
wherein the sensor is adapted to detect the indication of the concentration of the calibrating substance in the blood circulation, and
wherein the control component is adapted to facilitate the passage of the calibrating substance and the drug through the epithelial layer of the GI tract, responsively to the received indication.

74. The apparatus according to claim 71, wherein the sensor comprises a noninvasive external sensor.

75. The apparatus according to claim 71, wherein the sensor comprises an invasive sensor.

76. The apparatus according to claim 71, wherein the ingestible capsule is adapted to store the drug.

77. The apparatus according to claim 71, wherein the ingestible capsule is not adapted to contain the drug or to be assembled in an integral unit with the drug.

78. The apparatus according to claim 71, wherein the drug is contained in a drug pill, and wherein the ingestible capsule comprises a coupling mechanism, adapted to couple the drug pill to the ingestible capsule.

79. The apparatus according to claim 71,
wherein the ingestible capsule comprises an environmentally-sensitive mechanism, adapted to change a state thereof responsively to a disposition of the capsule within the GI tract, and
wherein the control component is adapted to facilitate the passage of the drug through the epithelial layer in response to a change of state of the environmentally-sensitive mechanism.

80. The apparatus according to claim 71,
wherein the indication includes respective first and second indications, sensed at respective first and second times,
wherein the wireless transmitter is adapted to transmit the first indication subsequent to the first time, and to transmit the second indication subsequent to the second time, and
wherein the control component is adapted to drive the first and second electrodes to apply first and second series of pulses, responsive to the first and second indications.

81. The apparatus according to claim 80, wherein the sensor unit is adapted to space the first and second times by at least 10 minutes.

82. The apparatus according to claim 80, wherein the control component is adapted to regulate a parameter of at least one of the series of pulses, responsive to at least one of the indications.

83. The apparatus according to claim 71,
wherein the ingestible capsule comprises a capsule wireless transmitter,
wherein the sensor unit comprises a sensor unit wireless receiver, and
wherein the ingestible capsule is adapted to wirelessly notify the sensor unit of a property of the capsule, via the capsule wireless transmitter and the sensor unit wireless receiver.

84. The apparatus according to claim 83, wherein the property is selected from the list consisting of: a location of the capsule, a status of the control component, a pH level of the GI tract, and a temperature of the GI tract, and wherein the capsule is adapted to wirelessly notify the sensor of the selected property.

85. The apparatus according to claim 71, wherein the substance includes a chemical, the blood concentration of which is affected by a blood concentration of the drug, and wherein the sensor is adapted to detect the indication of the concentration of the chemical in the blood circulation.

86. The apparatus according to claim 85, wherein the chemical is selected from the list consisting of: glucose, growth hormone, and hemoglobin-bound oxygen, and wherein the sensor is adapted to detect the indication of the concentration of the selected chemical in the blood circulation.

87. The apparatus according to claim 71, wherein the control component is adapted to apply the series of pulses at a current of between about 2 mA and about 4 mA.

88. The apparatus according to claim 87, wherein the control component is adapted to drive the first and second electrodes to apply the series of pulses at a current of about 3 mA.

89. The apparatus according to claim 71, wherein the control component is adapted to drive the first and second electrodes to apply the series of pulses at a frequency of between about 16 Hz and about 20 Hz.

90. The apparatus according to claim 89, wherein the control component is adapted to drive the first and second electrodes to apply the series of pulses at a frequency of about 18 Hz.

91. The apparatus according to claim 71, wherein the control component is adapted to drive the first and second electrodes to apply the series of pulses with a pulse duration of between about 0.5 milliseconds and about 1.5 milliseconds.

92. The apparatus according to claim 91, wherein the control component is adapted to drive the first and second electrodes to apply the series of pulses with a pulse duration of about 1 millisecond.

93. The apparatus according to claim 71, wherein the control component is adapted to drive the first and second electrodes to apply the series of pulses for a period of between about 1 and about 360 minutes.

94. The apparatus according to claim 93, wherein the control component is adapted to drive the first and second electrodes to apply the series of pulses for a period of between about 60 and about 240 minutes.

95. Apparatus for facilitating administration of a drug to a subject, the apparatus comprising:
a sensor unit, which comprises:
a sensor, adapted to detect an indication of a physiological parameter of the subject; and
a wireless transmitter, adapted to wirelessly transmit the indication; and
an ingestible capsule, which comprises:
a wireless receiver, adapted to receive the indication;
first and second electrodes; and
a control component, adapted to facilitate passage of the drug through an epithelial layer of a gastrointestinal (GI) tract of the subject by driving the first and second electrodes to apply a series of pulses at a current of less than about 5 mA, at a frequency of between about 12 Hz and about 24 Hz, and with a pulse duration of between about 0.5 milliseconds and about 3 milliseconds.

96. The apparatus according to claim 95, wherein the indication includes an indication of blood pressure of the subject, and wherein the sensor is adapted to sense the indication of blood pressure.

97. The apparatus according to claim 95, wherein the indication includes an indication of a heart-related parameter of the subject, and wherein the sensor is adapted to sense the indication of the heart-related parameter.

98. The apparatus according to claim 95, wherein the indication includes an indication of a level of activity of the subject, and wherein the sensor is adapted to sense the indication of the level of activity.

99. The apparatus according to claim 95, wherein the indication includes an indication of a temperature of the subject, and wherein the sensor is adapted to sense the indication of the temperature.

100. The apparatus according to claim 95, wherein the indication includes an indication of a circadian cycle of the subject, and wherein the sensor comprises clock circuitry adapted to sense the indication of the circadian cycle.

101. The apparatus according to claim 95, wherein the control component is adapted to apply the series of pulses at a current of between about 2 mA and about 4 mA.

102. The apparatus according to claim 101, wherein the control component is adapted to drive the first and second electrodes to apply the series of pulses at a current of about 3 mA.

103. The apparatus according to claim 95, wherein the control component is adapted to drive the first and second electrodes to apply the series of pulses at a frequency of between about 16 Hz and about 20 Hz.

104. The apparatus according to claim 103, wherein the control component is adapted to drive the first and second electrodes to apply the series of pulses at a frequency of about 18 Hz.

105. The apparatus according to claim 95, wherein the control component is adapted to drive the first and second electrodes to apply the series of pulses with a pulse duration of between about 0.5 milliseconds and about 1.5 milliseconds.

106. The apparatus according to claim 105, wherein the control component is adapted to drive the first and second electrodes to apply the series of pulses with a pulse duration of about 1 millisecond.

107. The apparatus according to claim 95, wherein the control component is adapted to drive the first and second electrodes to apply the series of pulses for a period of between about 1 and about 360 minutes.

108. The apparatus according to claim 107, wherein the control component is adapted to drive the first and second electrodes to apply the series of pulses for a period of between about 60 and about 240 minutes.

109. Apparatus for facilitating administration of a drug to a subject, the apparatus comprising:
first and second electrodes; and
a control component, adapted to facilitate passage of the drug through an epithelial layer of a gastrointestinal (GI) tract of the subject by driving the first and second electrodes to apply a series of pulses at a current of less than about 5 mA, at a frequency of between about 12 Hz and about 24 Hz, and with a pulse duration of between about 0.5 milliseconds and about 3 milliseconds.

110. The apparatus according to claim 109, wherein the control component is adapted to apply the series of pulses at a current of between about 2 mA and about 4 mA.

111. The apparatus according to claim 110, wherein the control component is adapted to drive the first and second electrodes to apply the series of pulses at a current of about 3 mA.

112. The apparatus according to claim 109, wherein the control component is adapted to drive the first and second electrodes to apply the series of pulses at a frequency of between about 16 Hz and about 20 Hz.

113. The apparatus according to claim 112, wherein the control component is adapted to drive the first and second electrodes to apply the series of pulses at a frequency of about 18 Hz.

114. The apparatus according to claim 109, wherein the control component is adapted to drive the first and second electrodes to apply the series of pulses with a pulse duration of between about 0.5 milliseconds and about 1.5 milliseconds.

115. The apparatus according to claim 114, wherein the control component is adapted to drive the first and second electrodes to apply the series of pulses with a pulse duration of about 1 millisecond.

116. The apparatus according to claim 109, wherein the control component is adapted to drive the first and second electrodes to apply the series of pulses for a period of between about 1 and about 360 minutes.

117. The apparatus according to claim 116, wherein the control component is adapted to drive the first and second electrodes to apply the series of pulses for a period of between about 60 and about 240 minutes.

118. A method for administration of a drug, comprising:
administering to a subject an ingestible capsule that includes the drug;
detecting a disposition of the capsule within a gastrointestinal (GI) tract of the subject; and
in response to detecting the disposition, facilitating, by the capsule, passage of the drug through an epithelial layer of the GI tract, by applying a series of pulses at a current of less than about 5 mA, at a frequency of between about 12 Hz and about 24 Hz, and with a pulse duration of between about 0.5 milliseconds and about 3 milliseconds.

119. The method according to claim 118, wherein the pulses include monophasic rectangular pulses, and wherein applying the series of pulses comprises applying the series of monophasic rectangular pulses.

120. The method according to claim 118,
wherein detecting the disposition of the capsule comprises sensing an indication of a distance traveled by the capsule in the GI tract, and
wherein facilitating the passage of the drug comprises facilitating the passage responsive to the distance.

121. The method according to claim 118, wherein detecting the disposition of the capsule comprises imaging the GI tract, and wherein applying the series of pulses comprises applying the series of pulses in response to an acquired image.

122. The method according to claim 118, wherein the disposition of the capsule includes a temperature in a vicinity of the capsule, wherein detecting the disposition of the capsule comprises sensing the temperature, and wherein applying the series of pulses comprises applying the series of pulses in response to the sensed temperature.

123. The method according to claim 118, wherein the disposition of the capsule includes a pH in a vicinity of the capsule, wherein detecting the disposition of the capsule comprises sensing the pH, and wherein applying the series of pulses comprises applying the series of pulses in response to the sensed pH.

124. The method according to claim 118, wherein detecting the disposition of the capsule comprises sensing a characteristic of the GI tract, and wherein applying the series of pulses comprises applying the series of pulses in response to the sensed characteristic.

125. The method according to claim 118, wherein facilitating the passage of the drug comprises applying the series of pulses, and applying an iontophoretic current.

126. The method according to claim 118, wherein applying the series of pulses comprises configuring the series of pulses using parameters selected at least in part responsively to the disposition of the capsule within the GI tract.

127. The method according to claim 118, wherein applying the series of pulses comprises configuring the series of pulses using parameters selected at least in part responsively to a property of the drug.

128. The method according to claim 118, wherein applying the series of pulses comprises applying the series of pulses at a current of between about 2 mA and about 4 mA.

129. The method according to claim 128, wherein applying the series of pulses comprises applying the series of pulses at a current of about 3 mA.

130. The method according to claim 118, wherein applying the series of pulses comprises applying the series of pulses at a frequency of between about 16 Hz and about 20 Hz.

131. The method according to claim 130, wherein applying the series of pulses comprises applying the series of pulses at a frequency of about 18 Hz.

132. The method according to claim 118, wherein applying the series of pulses comprises applying the series of pulses with a pulse duration of between about 0.5 milliseconds and about 1.5 milliseconds.

133. The method according to claim 132, wherein applying the series of pulses comprises applying the series of pulses with a pulse duration of about 1 millisecond.

134. The method according to claim 118, wherein applying the series of pulses comprises applying the series of pulses for a period of between about 1 and about 360 minutes.

135. The method according to claim 134, wherein applying the series of pulses comprises applying the series of pulses for a period of between about 60 and about 240 minutes.

136. A method for administration of a drug contained in a pill, comprising:
orally administering the pill to a subject;
orally administering to the subject an ingestible capsule that does not include the drug;
detecting a target location of the capsule within a gastrointestinal (GI) tract of the subject; and
in response to detecting the target location, facilitating, by the capsule, passage of the drug through an epithelial layer of the GI tract, by applying a series of pulses at a current of less than about 5 mA, at a frequency of between about 12 Hz and about 24 Hz, and with a pulse duration of between about 0.5 milliseconds and about 3 milliseconds.

137. The method according to claim 136, wherein detecting the target location of the capsule comprises sensing an indication of a distance traveled by the housing in the GI tract.

138. The method according to claim 136, wherein detecting the target location of the capsule comprises imaging the GI tract, and detecting the target location responsive to an acquired image.

139. The method according to claim 136, wherein detecting the target location comprises sensing a characteristic of the GI tract.

140. The method according to claim 136, wherein applying the series of pulses comprises applying the series of pulses at a current of between about 2 mA and about 4 mA.

141. The method according to claim 140, wherein applying the series of pulses comprises applying the series of pulses at a current of about 3 mA.

142. The method according to claim 136, wherein applying the series of pulses comprises applying the series of pulses at a frequency of between about 16 Hz and about 20 Hz.

143. The method according to claim 142, wherein applying the series of pulses comprises applying the series of pulses at a frequency of about 18 Hz.

144. The method according to claim 136, wherein applying the series of pulses comprises applying the series of pulses with a pulse duration of between about 0.5 milliseconds and about 1.5 milliseconds.

145. The method according to claim 144, wherein applying the series of pulses comprises applying the series of pulses with a pulse duration of about 1 millisecond.

146. The method according to claim 136, wherein applying the series of pulses comprises applying the series of pulses for a period of between about 1 and about 360 minutes.

147. The method according to claim 146, wherein applying the series of pulses comprises applying the series of pulses for a period of between about 60 and about 240 minutes.

148. A method for administration of a drug, comprising:
coupling, to an ingestible capsule, a drug pill containing the drug;
administering the capsule to a subject;
detecting a target location of the capsule within a gastrointestinal (GI) tract of the subject; and
in response to detecting the target location, facilitating, by the capsule, passage of the drug through an epithelial layer of the GI tract, by applying a series of pulses at a current of less than about 5 mA, at a frequency of between about 12 Hz and about 24 Hz, and with a pulse duration of between about 0.5 milliseconds and about 3 milliseconds.

149. The method according to claim 148, wherein the drug pill includes a commercially-available drug pill, and wherein coupling comprises coupling the commercially-available drug pill to the ingestible capsule.

150. The method according to claim 148, wherein coupling comprises coupling the drug pill to the ingestible capsule using an adhesive.

151. The method according to claim 148, wherein applying the series of pulses comprises driving a current between at least two electrodes, and wherein coupling comprises coupling the drug pill to the ingestible capsule using at least one of the electrodes.

152. The method according to claim 151, wherein coupling using the at least one of the electrodes comprises positioning the at least one of the electrodes around a portion of the drug pill.

153. The method according to claim 148, wherein applying the series of pulses comprises applying the series of pulses at a current of between about 2 mA and about 4 mA.

154. The method according to claim 153, wherein applying the series of pulses comprises applying the series of pulses at a current of about 3 mA.

155. The method according to claim 148, wherein applying the series of pulses comprises applying the series of pulses at a frequency of between about 16 Hz and about 20 Hz.

156. The method according to claim 155, wherein applying the series of pulses comprises applying the series of pulses at a frequency of about 18 Hz.

157. The method according to claim 148, wherein applying the series of pulses comprises applying the series of pulses with a pulse duration of between about 0.5 milliseconds and about 1.5 milliseconds.

158. The method according to claim 157, wherein applying the series of pulses comprises applying the series of pulses with a pulse duration of about 1 millisecond.

159. The method according to claim 148, wherein applying the series of pulses comprises applying the series of pulses for a period of between about 1 and about 360 minutes.

160. The method according to claim 159, wherein applying the series of pulses comprises applying the series of pulses for a period of between about 60 and about 240 minutes.

161. A method for facilitating administration of a drug to a subject, the method comprising:
administering an ingestible capsule to the subject;
detecting an indication of a concentration of a substance in a blood circulation of the subject;
wirelessly transmitting the indication;
receiving the indication at the ingestible capsule; and
responsively to the received indication, facilitating, by the capsule, passage of the drug through an epithelial layer of a gastrointestinal (GI) tract of the subject, by applying a series of pulses at a current of less than about 5 mA, at a frequency of between about 12 Hz and about 24 Hz, and with a pulse duration of between about 0.5 milliseconds and about 3 milliseconds.

162. The method according to claim 161, wherein the substance includes the drug, and wherein detecting the indication of the concentration of the substance comprises detecting the indication of the concentration of the drug in the blood circulation.

163. The method according to claim 161,
wherein the substance includes a calibrating substance,
wherein detecting the indication of the concentration of the substance comprises detecting the indication of the concentration of the calibrating substance in the blood circulation, and
wherein facilitating the passage of the drug comprises facilitating passage of the calibrating substance and the drug through the epithelial layer of the GI tract, responsively to the received indication.

164. The method according to claim 161, wherein detecting the indication comprises noninvasively detecting the indication.

165. The method according to claim 161, wherein detecting the indication comprises invasively detecting the indication.

166. The method according to claim 161, wherein the ingestible capsule includes the drug, and wherein administering the ingestible capsule comprises administering the ingestible capsule that includes the drug.

167. The method according to claim 161, wherein administering the ingestible capsule comprises administering an ingestible capsule that does not contain the drug and that is not assembled in an integral unit with the drug.

168. The method according to claim 161, wherein the drug is contained in a drug pill, and wherein administering the ingestible capsule comprises coupling the drug pill to the ingestible capsule.

169. The method according to claim 161, comprising detecting a disposition of the ingestible capsule within the GI tract, wherein facilitating the passage of the drug comprises facilitating the passage in response to detecting the disposition.

170. The method according to claim 161,
wherein the indication includes respective first and second indications,
wherein detecting the indication comprises detecting the first and second indications at respective first and second times,
wherein transmitting the indication comprises wirelessly transmitting the first indication subsequent to the first time, and wirelessly transmitting the second indication subsequent to the second time, and
wherein applying the series of pulses comprises applying first and second series of pulses, responsive to the first and second indications.

171. The method according to claim 170, wherein applying the series of pulses comprises regulating at least one of the series of pulses responsive to at least one of the indications.

172. The method according to claim 170, wherein detecting the first and second indications at the respective first and second times comprises spacing the first and second times by at least 10 minutes.

173. The method according to claim 161, comprising wirelessly transmitting, by the capsule, a property of the capsule.

174. The method according to claim 173, wherein the property is selected from the list consisting of: a location of the capsule, a status of the capsule, a pH of the GI tract, and a temperature of the GI tract, and wherein wirelessly transmitting the property comprises wirelessly transmitting, by the capsule, the selected property.

175. The method according to claim 161, wherein the substance includes a chemical, the blood concentration of which is affected by a blood concentration of the drug, and wherein detecting the indication of the concentration of the substance comprises detecting an indication of the concentration of the chemical in the blood circulation.

176. The method according to claim 175, wherein the chemical is selected from the list consisting of: glucose, growth hormone, and hemoglobin-bound oxygen, and wherein detecting the indication of the concentration of the chemical comprises detecting the indication of the concentration of the selected chemical in the blood circulation.

177. The method according to claim 161, wherein applying the series of pulses comprises applying the series of pulses at a current of between about 2 mA and about 4 mA.

178. The method according to claim 177, wherein applying the series of pulses comprises applying the series of pulses at a current of about 3 mA.

179. The method according to claim 161, wherein applying the series of pulses comprises applying the series of pulses at a frequency of between about 16 Hz and about 20 Hz.

180. The method according to claim 179, wherein applying the series of pulses comprises applying the series of pulses at a frequency of about 18 Hz.

181. The method according to claim 161, wherein applying the series of pulses comprises applying the series of pulses with a pulse duration of between about 0.5 milliseconds and about 1.5 milliseconds.

182. The method according to claim 181, wherein applying the series of pulses comprises applying the series of pulses with a pulse duration of about 1 millisecond.

183. The method according to claim 161, wherein applying the series of pulses comprises applying the series of pulses for a period of between about 1 and about 360 minutes.

184. The method according to claim 183, wherein applying the series of pulses comprises applying the series of pulses for a period of between about 60 and about 240 minutes.

185. A method for facilitating administration of a drug to a subject, the method comprising:
administering an ingestible capsule to the subject;
detecting an indication of a physiological parameter of the subject;
wirelessly transmitting the indication;
receiving the indication at the ingestible capsule; and
responsively to the received indication, facilitating, by the capsule, passage of the drug through an epithelial layer of a gastrointestinal (GI) tract of the subject, by applying a series of pulses at a current of less than about 5 mA, at a frequency of between about 12 Hz and about 24 Hz, and with a pulse duration of between about 0.5 milliseconds and about 3 milliseconds.

186. The method according to claim 185, wherein the indication includes an indication of blood pressure of the subject, and wherein detecting the indication comprises detecting the indication of blood pressure.

187. The method according to claim 185, wherein the indication includes an indication of a heart-related parameter of the subject, and wherein detecting the indication comprises detecting the indication of the heart-related parameter.

188. The method according to claim 185, wherein the indication includes an indication of a level of activity of the subject, and wherein detecting the indication comprises detecting the indication of the level of activity.

189. The method according to claim 185, wherein the indication includes an indication of a circadian cycle of the subject, and wherein detecting the indication comprises detecting the indication of the circadian cycle.

190. The method according to claim 189, wherein the drug includes an antithrombotic drug, and wherein facilitating the passage of the drug comprises facilitating the passage of the antithrombotic drug through the epithelial layer.

191. The method according to claim 185, wherein the indication includes an indication of a temperature of the subject, and wherein detecting the indication comprises detecting the indication of the temperature.

192. The method according to claim 191, wherein the drug includes an antibiotic, and wherein facilitating the passage of the drug comprises facilitating the passage of the antibiotic through the epithelial layer.

193. The method according to claim 185, wherein applying the series of pulses comprises applying the series of pulses at a current of between about 2 mA and about 4 mA.

194. The method according to claim 193, wherein applying the series of pulses comprises applying the series of pulses at a current of about 3 mA.

195. The method according to claim 185, wherein applying the series of pulses comprises applying the series of pulses at a frequency of between about 16 Hz and about 20 Hz.

196. The method according to claim 195, wherein applying the series of pulses comprises applying the series of pulses at a frequency of about 18 Hz.

197. The method according to claim 185, wherein applying the series of pulses comprises applying the series of pulses with a pulse duration of between about 0.5 milliseconds and about 1.5 milliseconds.

198. The method according to claim 197, wherein applying the series of pulses comprises applying the series of pulses with a pulse duration of about 1 millisecond.

199. The method according to claim 185, wherein applying the series of pulses comprises applying the series of pulses for a period of between about 1 and about 360 minutes.

200. The method according to claim 199, wherein applying the series of pulses comprises applying the series of pulses for a period of between about 60 and about 240 minutes.

201. A method for administration of a drug, comprising:
administering the drug to a gastrointestinal (GI) tract of a subject; and
facilitating passage of the drug through an epithelial layer of the GI tract by applying a series of pulses at a current of less than about 5 mA, at a frequency of between about 12 Hz and about 24 Hz, and with a pulse duration of between about 0.5 milliseconds and about 3 milliseconds.

202. The method according to claim 201, wherein applying the series of pulses comprises applying the series of pulses at a current of between about 2 mA and about 4 mA.

203. The method according to claim 202, wherein applying the series of pulses comprises applying the series of pulses at a current of about 3 mA.

204. The method according to claim 201, wherein applying the series of pulses comprises applying the series of pulses at a frequency of between about 16 Hz and about 20 Hz.

205. The method according to claim 204, wherein applying the series of pulses comprises applying the series of pulses at a frequency of about 18 Hz.

206. The method according to claim 201, wherein applying the series of pulses comprises applying the series of pulses with a pulse duration of between about 0.5 milliseconds and about 1.5 milliseconds.

207. The method according to claim 206, wherein applying the series of pulses comprises applying the series of pulses with a pulse duration of about 1 millisecond.

208. The method according to claim 201, wherein applying the series of pulses comprises applying the series of pulses for a period of between about 1 and about 360 minutes.

209. The method according to claim 208, wherein applying the series of pulses comprises applying the series of pulses for a period of between about 60 and about 240 minutes.
